SEQUENCE LISTING (160 sequences)

SEQ ID NO: 1 - protein, 126 aa, Artificial Sequence
  ankyrin repeat domain
GSDLGKKLLEAARAGQDDEVRILLAAGADVNAKDQYGITPLHLAAFRGHLEIVEVLLKHGAGVNADDMDGRTPLHPAAYKGHLEIVEVLLKNGADVNAQDKFGKTAFDISIDNGNEDLAEILQKLN

SEQ ID NO: 2 - protein, 159 aa, Artificial Sequence
  ankyrin repeat domain
GSDLGKKLLEAARAGQDDEVRILLAAGADINAKDKYGVTPLHLAAWAGHLEIVEVLLKAGADVNAKDQSGFTPLHLAAHRGHLEIVEVLLKAGADVNAKDYRGFTPLHLAAEDGHLEIVEVLLKAGADVNAQDKSGKTPADLAADAGHEDIAEVLQKLN

SEQ ID NO: 3 - protein, 159 aa, Artificial Sequence
  ankyrin repeat domain
GSDMGKKLLEAARAGQDDEVRILMANGADVNAFDQVGRTPLHLDALAGHLEIVEVLLKHGADVNAADMFGITPLHLAAFIGHLEIVEVLLKHGADVNAYDLDGRTPLHPAAYHGHLEIVEVLLKNGADVNAQDKFGKTAFDISIDNGNEDLAEILQKLN

SEQ ID NO: 4 - protein, 126 aa, Artificial Sequence
  ankyrin repeat domain
GSDLGHKLLEAATQGQDDEVRILLAAGADVNAKDEHGKTPLHLAAKKGHLEIVEVLLKAGADVNAKDDWGVTPLHLAAYWGHLEIVEVLLKAGADVNAQDNDGYTPADLAATFGHEDIAEVLQKLN

SEQ ID NO: 5 - protein, 126 aa, Artificial Sequence
  ankyrin repeat domain
GSDLGYTLLEAARRGQDDEVRELLKAGADVNAKDTIGYTPLHYAAQVGHLEIVEVLLKAGADVNAKDTQGITPLHVAAATGHLEIVEVLLKAGADVNAQDSKGRTPADLAAEQGHEDIAEVLQKLN

SEQ ID NO: 6 - protein, 159 aa, Artificial Sequence
  ankyrin repeat domain
GSDLGKKLLEAARAGQDDEVRELLKAGADVNAKDEWGKTPLHYAAALGHLEIVEVLLKAGADVNAKDQYGATPLHIAAFAGHLEIVEVLLKAGADVNAKDAWGFTPLHLAATRGHLEIVEVLLKAGADVNAQDKSGKTPADLAADAGHEDIVEVLQKLN

SEQ ID NO: 7 - protein, 159 aa, Artificial Sequence
  ankyrin repeat domain
GSDLGKKLLEAARAGQDDEVRELLKAGADVNAKDEWGKTPLHLAAALGHLEIVEVLLKAGADVNAKDAWGATPLHVAAFAGHLEIVEVLLKAGADVNAKDTIGFTPLHLAAHSGHLEIVEVLLKAGADVNAQDKSGKTPADLAADAGHEDIVEVLQKLN

SEQ ID NO: 8 - protein, 159 aa, Artificial Sequence
  ankyrin repeat domain
GSDLGKKLLEAARAGQDDEVRELLKAGADVNAKDQHGKTPLHYAAALGHLEIVEVLLKAGADVNAKDRWGATPLHIAAFAGHLEIVEVLLKAGADVNAKDEVGWTPLHLAARFGHLEIVEVLLKAGADVNAQDKSGKTPADLAADAGHEDIVEVLQKLN

SEQ ID NO: 9 - protein, 126 aa, Artificial Sequence
  ankyrin repeat domain
GSDLGKKLLEAARAGQDDEVRILMANGADVNAFDTTGRTPLHLAALWGHLEIVEVLLKHGADVNASDWFSITPLHLAAFMGHLEIVEVLLNHGADVNAQDKFGKTAFDISIDHGNEDLAEILQKLN

SEQ ID NO: 10 - protein, 126 aa, Artificial Sequence
  ankyrin repeat domain
GSDLGKKLLEATRAGQDDEVRILLAAGADVNAKDEYGITPLHLAAFRGHLEIVEVLLKHGADVNAIDYNGRTPLHPAAYSGHLEIVEVLLKYGADVNAQDKFGKTAFDISIDNGNEDLAEILQKLN

SEQ ID NO: 11 - protein, 159 aa, Artificial Sequence
  ankyrin repeat domain
GSDLGKKLLEAARAGQDDEVRILLAAGADVNAKDTWGWTPLHLAAKSGHLEIVEVLLKAGADVNAKDYHGVTPLHLATFWGHLEIVEVLLKAGADVNAKDWYGETPLHLAAIRGHLEIVEVLLKAGADVNAQDKSGKTPADLAADAGHEDIAEVLQKLN

SEQ ID NO: 12 - protein, 159 aa, Artificial Sequence
  ankyrin repeat domain
GSDLGKKLLEAARAGQDDEVRILLAAGADVSAKDQWGWTPLHLAARVGHLEIVEVLLKAGADVNAKDVRGVTPLHLAAFHGHLEIVEVLLKAGADVNAKDYAGHTPLHLAAWQGHLEIIEDLLKAGADVNAQDKSGKTPADLAADAGHEDIAEVLQKLN

SEQ ID NO: 13 - protein, 159 aa, Artificial Sequence
  ankyrin repeat domain
GSDLGKKLLEAARAGQDDEVRILLAAGADVNAKDDWGWTPLHLAARHGHLEIVEVLLKAGTDVNAKDKRGVTPLHLAAYIGHLEIAEVLLKAGADVNAKDFQGWTPLHLAAVRGHLEIVEVLLKAGADVNAQDKSGKTPADLAADAGHEDIAEVLQKLN

SEQ ID NO: 14 - protein, 159 aa, Artificial Sequence
  ankyrin repeat domain
GSDLGKKLLEAARAGQDDEVRILLAAGADVNAKDYSGRTPLHLAALTGHLEIVKVLLKHGADVNASDDYGITPLHLDAFIGHLEIVEVLLKNGADVNAMDYQGRTPLHPAAYTGHLEIVEVLLKYGADVNAQDKFGKTAFDISIDNGNEDLAEILQKLN

SEQ ID NO: 15 - protein, 159 aa, Artificial Sequence
  ankyrin repeat domain
GSDLGKKLLEAARAGQDDEVRILMANGADVNAFDAIGRTPLHLAALYGHLEIVEVLLKHVADVNATDEYGITPLHLAAFMGHLEIVEVLLKYGADVNAIDFDGRTPLHPAAYRGHLEIVEVLLKHGADVNAQDKFGKTAFDISIDNGNEDLAEILQKLN

SEQ ID NO: 16 - protein, 159 aa, Artificial Sequence
  ankyrin repeat domain
GSDLGKKLLEAARAGQDDEVRILMANGTDVNATDMTGRTPLHLAALYGHLEIVEVLLKYGADVNALDKFGITPLHLAAFMGHLEIVEVLLKYGADVNAADLDGRTPLHPTAYRGHLEIVEVLLKNGADVNAQDKFGKTAFDISIDNGNEDLAEILQKLN

SEQ ID NO: 17 - protein, 159 aa, Artificial Sequence
  ankyrin repeat domain
GSDLGKKLLEAARAGQDDEVRILMANGADVNATDLMGRTPLHLAASRGHLEIVEVLLKNGADVNADDKYGITSLHLAAFLGHLEIVEVLLKYGADVNAIDFDGRTPLHPAAYRGHLEIVEVLLKHGADVNAQDKFGQTAFDISIDNGNEDLAEILQKLN

SEQ ID NO: 18 - protein, 159 aa, Artificial Sequence
  ankyrin repeat domain
GSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKLN

SEQ ID NO: 19 - protein, 126 aa, Artificial Sequence
  ankyrin repeat domain
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKLN

SEQ ID NO: 20 - protein, 126 aa, Artificial Sequence
  ankyrin repeat domain
GSDLGEKLLIAALFGQDEEVRILLAAGADVNAKDEWGHTPLHKAALSGHLEIVEVLLKAGADVNAKDQKGSTPLHWAATRGHLEIVEVLLKAGADVNAQDHIGRTPADLAANFGHEDIAEVLQKLN

SEQ ID NO: 21 - protein, 126 aa, Artificial Sequence
  ankyrin repeat domain
GSDLGQKLLNAAFIGQDDEVRILLAAGAGVNAKDRHGHTPLHVTAHHGHLEIVEVLLKAGTDVNAKDNQGNTPLHVAAEADHLEIVEVLLKAGADVNAQDAHGRTPADLAAQLGHEDIAEVLQKLN

SEQ ID NO: 22 - protein, 126 aa, Artificial Sequence
  ankyrin repeat domain
GSDLGDKLLTAANYGQDDEVRILLAAGADVNAKDRFGHTPLHVAAHFGHLEIVEVLLKAGADVNAKDSLGHTPLHLAAEAGHLEIVEVLLKAGADVNAQDFHGITPADLAAHQGHEDIAEVLQKLN

SEQ ID NO: 23 - protein, 126 aa, Artificial Sequence
  ankyrin repeat domain
GSDLGLKLLSAAQVGQDDEVRILLAAGADVNVKDQFGHTPLHVATHFGHLEIVEVLLKAGADVNAKDALGHTPLHLAAEAGHLEIVEVLLKAGADVNAQDAHGRTPADLAAQLGHEDIAEVLQKLN

SEQ ID NO: 24 - protein, 126 aa, Artificial Sequence
  ankyrin repeat domain
GSDLGEKLLEAARRGQDDEVRELLKAGADVNAKDRYGITPLHLAAFLGHLEIVEVLLKDGADVNAKDEWGATPLHVAAYKGHLEIVEVLLKAGADVNAQDLYGKTPADLAAEQGHEDIEEVLQKLN

SEQ ID NO: 25 - protein, 126 aa, Artificial Sequence
  ankyrin repeat domain
GSDLGYKLLEAARRGQDDEVRELLKAGADVNAKDVIGYTPLHYAASEGHLEIVEVLLKAGADVNAKDTKGITPLHVAASTGHLEIVEVLLKAGADVNAQDLRGYTPADLAARQGHEDIGEVLQKLN

SEQ ID NO: 26 - protein, 159 aa, Artificial Sequence
  ankyrin repeat domain
GSDLGKKLLEAARAGQDDEVRELLKAGADVNAKDEVGRTPLHLAAQIGHLEIVEVLLKAGADVNAKNRWGVTPLHVAAWVGHLEIVEVLLKAGADVNAKDQQGATPLHLAAIRGHLEIVEVLLKAGADVNAQDKSGKTPADLAADAGHEDIAEVLQKLN

SEQ ID NO: 27 - protein, 159 aa, Artificial Sequence
  ankyrin repeat domain
GSDLGKKLLEAARAGQDDEVRELLKAGADVNAKDEWDKTPLHYAAALGHLEIVEVLLKAGADVNAKDVWGATPLHIAAFAGHLEIVEVLLKAGADVNAKDTLGYTPLHLAATRGHLEIVEALLKAGADVNAQDKSGKTPADLAADVGHEDIAEVLQKLN

SEQ ID NO: 28 - protein, 159 aa, Artificial Sequence
  ankyrin repeat domain
GSDLGKKLLEAARAGQDDEVRELLKAGADVNAKDEWGKTPLHYAAALGHLEIVEVLLKAGADVNAKDRWGATPLHIAAFAGHLEIVEVLLKAGADVNAKDEVGWTPLHLAARSGHLEIVEVLLKVGADVNAQDKSGKTPADLAADAGHEDIVEVLQKLN

SEQ ID NO: 29 - protein, 159 aa, Artificial Sequence
  ankyrin repeat domain
GSDLGKKLLEAARAGQDDEVRELLKAGADVNAKDTIGRTPLHIAARYGHLEIVEVLLKAGADVNAKDVYDITPLHLAAFLGHLEIVEVLLKAGADVNAKDYEGTTPLHVAAWHGHLEIVEVLLKAGADVNAQDKSGKTPADLAADAGHEDIAEVLQKLN

SEQ ID NO: 30 - protein, 159 aa, Artificial Sequence
  ankyrin repeat domain
GSDMGKKLLEAARAGQDDEVRELLKAGADVNAKDEWGWTPLHVAAERGHLEIVEVLLKAGADVNAKDKHGVTPLHLAAWRGHLEIVEVLLKAGADVNAKDIQGLTPLHIAAHQGHLEIVEVLLKAGADVNAQDKSGKTPADLAADAGHEDIAEVLQKLN

SEQ ID NO: 31 - protein, 159 aa, Artificial Sequence
  ankyrin repeat domain
GSDLGKKLLEAARAGQDDEVRELLKAGADVNAKDEFGKTPLHLAAALGHLEIVEVLLKAGADVNAKGLWGETPLHVAAFAGHLEIVEVLLKAGADVNAKDTYGYTPLHLAASRGHLEIVEVLLKAGADVNAQDKSGKTPADLAADAGHEDIAEVLQKLN

SEQ ID NO: 32 - protein, 159 aa, Artificial Sequence
  ankyrin repeat domain
GSDLGKKLLEAARAGQDDEVRELLKAGADVNAKDEWDKTPLHLAAALDHLEIVEVLLKAGADVNAKDQYGATPLHVAAFAGHLEIVEVLLKAGADVNAKDLFGWTPLHLAALRGHLEIVEVLLKAGADVNAQDKSGKTPADLAADAGHEDIVEVLQKLN

SEQ ID NO: 33 - protein, 159 aa, Artificial Sequence
  ankyrin repeat domain
GSDLGKKLLEAARAGQDDEVRELLKAGADVNAKDEWGKTPLHLAAIRGHLEIVEVLLKAGADVNAKDTHGITPLHLAAFVGHLEIVEVLLKAGADVNAKDYLGRTPLHIAAAKGHLEIVEVLLKAGADVNAQDKSGKTPADLAADAGHEDIVEVLQKLN

SEQ ID NO: 34 - protein, 159 aa, Artificial Sequence
  ankyrin repeat domain
GSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 35 - protein, 126 aa, Artificial Sequence
  ankyrin repeat domain
GSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAA

SEQ ID NO: 36 - protein, 10 aa, Artificial Sequence
  His-tag
MRGSHHHHHH

SEQ ID NO: 37 - protein, 21 aa, Artificial Sequence
  His-HA-tag
MRGSHHHHHHGSYPYDVPDYA

SEQ ID NO: 38 - protein, 126 aa, Artificial Sequence
  ankyrin repeat domain
GSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 39 - protein, 24 aa, Artificial Sequence
  PT-linker
GSPTPTPTTPTPTPTTPTPTPTGS

SEQ ID NO: 40 - protein, 307 aa, Artificial Sequence
  protein comprising two ankyrin repeat domains
GSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGSPTPTPTTPTPTPTTPTPTPTGSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 41 - protein, 274 aa, Artificial Sequence
  protein comprising two ankyrin repeat domains
GSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGSPTPTPTTPTPTPTTPTPTPTGSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAA

SEQ ID NO: 42 - protein, 307 aa, Artificial Sequence
  protein comprising two ankyrin repeat domains
GSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDEVGRTPLHLAAQIGHLEIVEVLLKAGADVNAKNRWGVTPLHVAAWVGHLEIVEVLLKAGADVNAKDQQGATPLHLAAIRGHLEIVEVLLKAGADVNAQDKSGKTPADLAADAGHEDIAEVLQKAA

SEQ ID NO: 43 - protein, 274 aa, Artificial Sequence
  protein comprising two ankyrin repeat domains
GSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 44 - protein, 126 aa, Artificial Sequence
  ankyrin repeat domain
GSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 45 - protein, 741 aa, Artificial Sequence
  His-tagged extracellular domain of human FAP
HHHHHHLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 46 - protein, 741 aa, Artificial Sequence
  His-tagged extracellular domain of cynomolgus FAP
HHHHHHLRPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 47 - protein, 736 aa, Artificial Sequence
  extracellular domain of mouse FAP
LRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 48 - protein, 33 aa, Artificial Sequence
  ankyrin repeat module
KDQYGITPLHLAAFRGHLEIVEVLLKHGAGVNA

SEQ ID NO: 49 - protein, 33 aa, Artificial Sequence
  ankyrin repeat module
DDMDGRTPLHPAAYKGHLEIVEVLLKNGADVNA

SEQ ID NO: 50 - protein, 33 aa, Artificial Sequence
  ankyrin repeat module
KDKYGVTPLHLAAWAGHLEIVEVLLKAGADVNA

SEQ ID NO: 51 - protein, 33 aa, Artificial Sequence
  ankyrin repeat module
KDQSGFTPLHLAAHRGHLEIVEVLLKAGADVNA

SEQ ID NO: 52 - protein, 33 aa, Artificial Sequence
  ankyrin repeat module
KDYRGFTPLHLAAEDGHLEIVEVLLKAGADVNA

SEQ ID NO: 53 - protein, 33 aa, Artificial Sequence
  ankyrin repeat module
FDQVGRTPLHLDALAGHLEIVEVLLKHGADVNA

SEQ ID NO: 54 - protein, 33 aa, Artificial Sequence
  ankyrin repeat module
ADMFGITPLHLAAFIGHLEIVEVLLKHGADVNA

SEQ ID NO: 55 - protein, 33 aa, Artificial Sequence
  ankyrin repeat module
YDLDGRTPLHPAAYHGHLEIVEVLLKNGADVNA

SEQ ID NO: 56 - protein, 33 aa, Artificial Sequence
  ankyrin repeat module
KDEHGKTPLHLAAKKGHLEIVEVLLKAGADVNA

SEQ ID NO: 57 - protein, 33 aa, Artificial Sequence
  ankyrin repeat module
KDDWGVTPLHLAAYWGHLEIVEVLLKAGADVNA

SEQ ID NO: 58 - protein, 33 aa, Artificial Sequence
  ankyrin repeat module
KDTIGYTPLHYAAQVGHLEIVEVLLKAGADVNA

SEQ ID NO: 59 - protein, 33 aa, Artificial Sequence
  ankyrin repeat module
KDTQGITPLHVAAATGHLEIVEVLLKAGADVNA

SEQ ID NO: 60 - protein, 33 aa, Artificial Sequence
  ankyrin repeat module
KDEWGKTPLHYAAALGHLEIVEVLLKAGADVNA

SEQ ID NO: 61 - protein, 33 aa, Artificial Sequence
  ankyrin repeat module
KDQYGATPLHIAAFAGHLEIVEVLLKAGADVNA

SEQ ID NO: 62 - protein, 33 aa, Artificial Sequence
  ankyrin repeat module
KDAWGFTPLHLAATRGHLEIVEVLLKAGADVNA

SEQ ID NO: 63 - protein, 34 aa, Artificial Sequence
  ankyrin repeat module
CKDEWGKTPLHLAAALGHLEIVEVLLKAGADVNA

SEQ ID NO: 64 - protein, 33 aa, Artificial Sequence
  ankyrin repeat module
KDAWGATPLHVAAFAGHLEIVEVLLKAGADVNA

SEQ ID NO: 65 - protein, 33 aa, Artificial Sequence
  ankyrin repeat module
KDTIGFTPLHLAAHSGHLEIVEVLLKAGADVNA

SEQ ID NO: 66 - protein, 33 aa, Artificial Sequence
  ankyrin repeat module
KDQHGKTPLHYAAALGHLEIVEVLLKAGADVNA

SEQ ID NO: 67 - protein, 33 aa, Artificial Sequence
  ankyrin repeat module
KDRWGATPLHIAAFAGHLEIVEVLLKAGADVNA

SEQ ID NO: 68 - protein, 33 aa, Artificial Sequence
  ankyrin repeat module
KDEVGWTPLHLAARFGHLEIVEVLLKAGADVNA

SEQ ID NO: 69 - protein, 33 aa, Artificial Sequence
  ankyrin repeat module
FDTTGRTPLHLAALWGHLEIVEVLLKHGADVNA

SEQ ID NO: 70 - protein, 33 aa, Artificial Sequence
  ankyrin repeat module
SDWFSITPLHLAAFMGHLEIVEVLLNHGADVNA

SEQ ID NO: 71 - protein, 33 aa, Artificial Sequence
  ankyrin repeat module
KDEYGITPLHLAAFRGHLEIVEVLLKHGADVNA

SEQ ID NO: 72 - protein, 33 aa, Artificial Sequence
  ankyrin repeat module
IDYNGRTPLHPAAYSGHLEIVEVLLKYGADVNA

SEQ ID NO: 73 - protein, 33 aa, Artificial Sequence
  ankyrin repeat module
KDTWGWTPLHLAAKSGHLEIVEVLLKAGADVNA

SEQ ID NO: 74 - protein, 33 aa, Artificial Sequence
  ankyrin repeat module
KDYHGVTPLHLATFWGHLEIVEVLLKAGADVNA

SEQ ID NO: 75 - protein, 33 aa, Artificial Sequence
  ankyrin repeat module
KDWYGETPLHLAAIRGHLEIVEVLLKAGADVNA

SEQ ID NO: 76 - protein, 33 aa, Artificial Sequence
  ankyrin repeat module
KDQWGWTPLHLAARVGHLEIVEVLLKAGADVNA

SEQ ID NO: 77 - protein, 33 aa, Artificial Sequence
  ankyrin repeat module
KDVRGVTPLHLAAFHGHLEIVEVLLKAGADVNA

SEQ ID NO: 78 - protein, 158 aa, Artificial Sequence
  ankyrin repeat module
CDLGKKLLEAARAGQDDEVRILLAAGADVSAKDQWGWTPLHLAARVGHLEIVEVLLKAGADVNAKDVRGVTPLHLAAFHGHLEIVEVLLKAGADVNAKDYAGHTPLHLAAWQGHLEIIEDLLKAGADVNAQDKSGKTPADLAADAGHEDIAEVLQKLN

SEQ ID NO: 79 - protein, 33 aa, Artificial Sequence
  ankyrin repeat module
KDDWGWTPLHLAARHGHLEIVEVLLKAGTDVNA

SEQ ID NO: 80 - protein, 33 aa, Artificial Sequence
  ankyrin repeat module
KDKRGVTPLHLAAYIGHLEIAEVLLKAGADVNA

SEQ ID NO: 81 - protein, 33 aa, Artificial Sequence
  ankyrin repeat module
KDFQGWTPLHLAAVRGHLEIVEVLLKAGADVNA

SEQ ID NO: 82 - protein, 33 aa, Artificial Sequence
  ankyrin repeat module
KDYSGRTPLHLAALTGHLEIVKVLLKHGADVNA

SEQ ID NO: 83 - protein, 33 aa, Artificial Sequence
  ankyrin repeat module
SDDYGITPLHLDAFIGHLEIVEVLLKNGADVNA

SEQ ID NO: 84 - protein, 33 aa, Artificial Sequence
  ankyrin repeat module
MDYQGRTPLHPAAYTGHLEIVEVLLKYGADVNA

SEQ ID NO: 85 - protein, 33 aa, Artificial Sequence
  ankyrin repeat module
FDAIGRTPLHLAALYGHLEIVEVLLKHVADVNA

SEQ ID NO: 86 - protein, 33 aa, Artificial Sequence
  ankyrin repeat module
TDEYGITPLHLAAFMGHLEIVEVLLKYGADVNA

SEQ ID NO: 87 - protein, 33 aa, Artificial Sequence
  ankyrin repeat module
IDFDGRTPLHPAAYRGHLEIVEVLLKHGADVNA

SEQ ID NO: 88 - protein, 33 aa, Artificial Sequence
  ankyrin repeat module
TDMTGRTPLHLAALYGHLEIVEVLLKYGADVNA

SEQ ID NO: 89 - protein, 33 aa, Artificial Sequence
  ankyrin repeat module
LDKFGITPLHLAAFMGHLEIVEVLLKYGADVNA

SEQ ID NO: 90 - protein, 33 aa, Artificial Sequence
  ankyrin repeat module
ADLDGRTPLHPTAYRGHLEIVEVLLKNGADVNA

SEQ ID NO: 91 - protein, 33 aa, Artificial Sequence
  ankyrin repeat module
TDLMGRTPLHLAASRGHLEIVEVLLKNGADVNA

SEQ ID NO: 92 - protein, 33 aa, Artificial Sequence
  ankyrin repeat module
DDKYGITSLHLAAFLGHLEIVEVLLKYGADVNA

SEQ ID NO: 93 - protein, 33 aa, Artificial Sequence; identical to SEQ ID NO: 87
  ankyrin repeat module

SEQ ID NO: 94 - protein, 33 aa, Artificial Sequence
  ankyrin repeat module
KDVLGWTPLHLAAFEGHLEIVEVLLKAGADVNA

SEQ ID NO: 95 - protein, 33 aa, Artificial Sequence
  ankyrin repeat module
KDKKGWTPLQLAARTGHLEIVEVLLKAGADVNA

SEQ ID NO: 96 - protein, 33 aa, Artificial Sequence
  ankyrin repeat module
KDHIGATPLHLAAWQGHPEIVEVLLKAGADVNA

SEQ ID NO: 97 - protein, 33 aa, Artificial Sequence
  ankyrin repeat module
KDQWGLTPLHKAALQGHLEIVEVLLKAGADVNA

SEQ ID NO: 98 - protein, 33 aa, Artificial Sequence
  ankyrin repeat module
KDERGHTPLHWAARFGHLEIVEVLLKAGADVNA

SEQ ID NO: 99 - protein, 33 aa, Artificial Sequence
  ankyrin repeat module
KDEWGHTPLHKAALSGHLEIVEVLLKAGADVNA

SEQ ID NO: 100 - protein, 33 aa, Artificial Sequence
  ankyrin repeat module
KDQKGSTPLHWAATRGHLEIVEVLLKAGADVNA

SEQ ID NO: 101 - protein, 33 aa, Artificial Sequence
  ankyrin repeat module
KDRHGHTPLHVTAHHGHLEIVEVLLKAGTDVNA

SEQ ID NO: 102 - protein, 33 aa, Artificial Sequence
  ankyrin repeat module
KDNQGNTPLHVAAEADHLEIVEVLLKAGADVNA

SEQ ID NO: 103 - protein, 33 aa, Artificial Sequence
  ankyrin repeat module
KDRFGHTPLHVAAHFGHLEIVEVLLKAGADVNA

SEQ ID NO: 104 - protein, 33 aa, Artificial Sequence
  ankyrin repeat module
KDSLGHTPLHLAAEAGHLEIVEVLLKAGADVNA

SEQ ID NO: 105 - protein, 33 aa, Artificial Sequence
  ankyrin repeat module
KDQFGHTPLHVATHFGHLEIVEVLLKAGADVNA

SEQ ID NO: 106 - protein, 33 aa, Artificial Sequence
  ankyrin repeat module
KDALGHTPLHLAAEAGHLEIVEVLLKAGADVNA

SEQ ID NO: 107 - protein, 33 aa, Artificial Sequence
  ankyrin repeat module
KDRYGITPLHLAAFLGHLEIVEVLLKDGADVNA

SEQ ID NO: 108 - protein, 33 aa, Artificial Sequence
  ankyrin repeat module
KDEWGATPLHVAAYKGHLEIVEVLLKAGADVNA

SEQ ID NO: 109 - protein, 33 aa, Artificial Sequence
  ankyrin repeat module
KDVIGYTPLHYAASEGHLEIVEVLLKAGADVNA

SEQ ID NO: 110 - protein, 33 aa, Artificial Sequence
  ankyrin repeat module
KDTKGITPLHVAASTGHLEIVEVLLKAGADVNA

SEQ ID NO: 111 - protein, 33 aa, Artificial Sequence
  ankyrin repeat module
KDEVGRTPLHLAAQIGHLEIVEVLLKAGADVNA

SEQ ID NO: 112 - protein, 33 aa, Artificial Sequence
  ankyrin repeat module
KNRWGVTPLHVAAWVGHLEIVEVLLKAGADVNA

SEQ ID NO: 113 - protein, 33 aa, Artificial Sequence
  ankyrin repeat module
KDQQGATPLHLAAIRGHLEIVEVLLKAGADVNA

SEQ ID NO: 114 - protein, 33 aa, Artificial Sequence
  ankyrin repeat module
KDEWDKTPLHYAAALGHLEIVEVLLKAGADVNA

SEQ ID NO: 115 - protein, 33 aa, Artificial Sequence
  ankyrin repeat module
KDVWGATPLHIAAFAGHLEIVEVLLKAGADVNA

SEQ ID NO: 116 - protein, 33 aa, Artificial Sequence
  ankyrin repeat module
KDTLGYTPLHLAATRGHLEIVEALLKAGADVNA

SEQ ID NO: 117 - protein, 33 aa, Artificial Sequence; identical to SEQ ID NO: 60
  ankyrin repeat module

SEQ ID NO: 118 - protein, 33 aa, Artificial Sequence; identical to SEQ ID NO: 67
  ankyrin repeat module

SEQ ID NO: 119 - protein, 33 aa, Artificial Sequence
  ankyrin repeat module
KDEVGWTPLHLAARSGHLEIVEVLLKVGADVNA

SEQ ID NO: 120 - protein, 33 aa, Artificial Sequence
  ankyrin repeat module
KDTIGRTPLHIAARYGHLEIVEVLLKAGADVNA

SEQ ID NO: 121 - protein, 33 aa, Artificial Sequence
  ankyrin repeat module
KDVYDITPLHLAAFLGHLEIVEVLLKAGADVNA

SEQ ID NO: 122 - protein, 33 aa, Artificial Sequence
  ankyrin repeat module
KDYEGTTPLHVAAWHGHLEIVEVLLKAGADVNA

SEQ ID NO: 123 - protein, 33 aa, Artificial Sequence
  ankyrin repeat module
KDEWGWTPLHVAAERGHLEIVEVLLKAGADVNA

SEQ ID NO: 124 - protein, 33 aa, Artificial Sequence
  ankyrin repeat module
KDKHGVTPLHLAAWRGHLEIVEVLLKAGADVNA

SEQ ID NO: 125 - protein, 33 aa, Artificial Sequence
  ankyrin repeat module
KDIQGLTPLHIAAHQGHLEIVEVLLKAGADVNA

SEQ ID NO: 126 - protein, 33 aa, Artificial Sequence
  ankyrin repeat module
KDEFGKTPLHLAAALGHLEIVEVLLKAGADVNA

SEQ ID NO: 127 - protein, 33 aa, Artificial Sequence
  ankyrin repeat module
KGLWGETPLHVAAFAGHLEIVEVLLKAGADVNA

SEQ ID NO: 128 - protein, 33 aa, Artificial Sequence
  ankyrin repeat module
KDTYGYTPLHLAASRGHLEIVEVLLKAGADVNA

SEQ ID NO: 129 - protein, 33 aa, Artificial Sequence
  ankyrin repeat module
KDEWDKTPLHLAAALDHLEIVEVLLKAGADVNA

SEQ ID NO: 130 - protein, 33 aa, Artificial Sequence
  ankyrin repeat module
KDQYGATPLHVAAFAGHLEIVEVLLKAGADVNA

SEQ ID NO: 131 - protein, 33 aa, Artificial Sequence
  ankyrin repeat module
KDLFGWTPLHLAALRGHLEIVEVLLKAGADVNA

SEQ ID NO: 132 - protein, 33 aa, Artificial Sequence
  ankyrin repeat module
KDEWGKTPLHLAAIRGHLEIVEVLLKAGADVNA

SEQ ID NO: 133 - protein, 33 aa, Artificial Sequence
  ankyrin repeat module
KDTHGITPLHLAAFVGHLEIVEVLLKAGADVNA

SEQ ID NO: 134 - protein, 33 aa, Artificial Sequence
  ankyrin repeat module
KDYLGRTPLHIAAAKGHLEIVEVLLKAGADVNA

SEQ ID NO: 135 - protein, 32 aa, Artificial Sequence
  N-cap
GSDLGKKLLEAARAGQDDEVRILLAAGADVNA

SEQ ID NO: 136 - protein, 32 aa, Artificial Sequence
  N-cap
GSDLGKKLLEAARAGQDDEVRELLKAGADVNA

SEQ ID NO: 137 - protein, 32 aa, Artificial Sequence
  N-cap
GSDLGKKLLEAARAGQDDEVRILLKAGADVNA

SEQ ID NO: 138 - protein, 32 aa, Artificial Sequence
  AR sequence motif
  MISC_FEATURE (6)..(6): Xaa can be any naturally occurring amino acid
  MISC_FEATURE (10)..(10): Xaa can be any naturally occurring amino acid
  MISC_FEATURE (13)..(14): Xaa can be any naturally occurring amino acid
GSDLGXKLLXAAXXGQDDEVRILLAAGADVNA

SEQ ID NO: 139 - protein, 33 aa, Artificial Sequence
  AR sequence motif
  MISC_FEATURE (1)..(1): Xaa can be any naturally occurring amino acid
  MISC_FEATURE (3)..(4): Xaa can be any naturally occurring amino acid
  MISC_FEATURE (6)..(6): Xaa can be any naturally occurring amino acid
  MISC_FEATURE (14)..(15): Xaa can be any naturally occurring amino acid
  MISC_FEATURE (27)..(27): Xaa can be any naturally occurring amino acid
XDXXGXTPLHLAAXXGHLEIVEVLLKXGADVNA

SEQ ID NO: 140 - protein, 33 aa, Artificial Sequence
  AR sequence motif
  MISC_FEATURE (3)..(4): Xaa can be any naturally occurring amino acid
  MISC_FEATURE (6)..(6): Xaa can be any naturally occurring amino acid
  MISC_FEATURE (14)..(15): Xaa can be any naturally occurring amino acid
KDXXGXTPLHLAAXXGHLEIVEVLLKAGADVNA

SEQ ID NO: 141 - protein, 33 aa, Artificial Sequence
  AR sequence motif
  MISC_FEATURE (3)..(4): Xaa can be any naturally occurring amino acid
  MISC_FEATURE (6)..(6): Xaa can be any naturally occurring amino acid
  MISC_FEATURE (11)..(11): Xaa can be any naturally occurring amino acid
  misc_feature (14)..(14): Xaa can be any naturally occurring amino acid
  MISC_FEATURE (15)..(15): Xaa can be any naturally occurring amino acid
KDXXGXTPLHXAAXXGHLEIVEVLLKAGADVNA

SEQ ID NO: 142 - protein, 28 aa, Artificial Sequence
  capping module
QDKFGKTPADIAADNGHEDIAEVLQKLN

SEQ ID NO: 143 - protein, 28 aa, Artificial Sequence
  AR sequence motif
  MISC_FEATURE (3)..(4): Xaa can be any naturally occurring amino acid
  MISC_FEATURE (6)..(6): Xaa can be any naturally occurring amino acid
  MISC_FEATURE (14)..(15): Xaa can be any naturally occurring amino acid
QDXXGXTPADLAAXXGHEDIAEVLQKLN

SEQ ID NO: 144 - protein, 159 aa, Artificial Sequence
  ankyrin repeat domain
GSDLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKLN

SEQ ID NO: 145 - protein, 159 aa, Artificial Sequence
  ankyrin repeat domain
GSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKLN

SEQ ID NO: 146 - protein, 159 aa, Artificial Sequence
  ankyrin repeat domain
GSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 147 - protein, 159 aa, Artificial Sequence
  ankyrin repeat domain
GSDLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 148 - protein, 159 aa, Artificial Sequence
  ankyrin repeat domain
GSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKLN

SEQ ID NO: 149 - protein, 159 aa, Artificial Sequence
  ankyrin repeat domain
GSDLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKLN

SEQ ID NO: 150 - protein, 159 aa, Artificial Sequence
  ankyrin repeat domain
GSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKLN

SEQ ID NO: 151 - protein, 159 aa, Artificial Sequence
  ankyrin repeat domain
GSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 152 - protein, 159 aa, Artificial Sequence
  ankyrin repeat domain
GSDLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 153 - protein, 159 aa, Artificial Sequence
  ankyrin repeat domain
GSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 154 - protein, 585 aa, Homo sapiens [sequence omitted]

SEQ ID NO: 155 - protein, 5 aa, Artificial Sequence
  Consensus GS-linker
  REPEAT (1)..(5): [Gly-Gly-Gly-Gly-Ser]n, wherein n is 1, 2, 3, 4, 5, or 6
GGGGS

SEQ ID NO: 156 - DNA, 477 bp, Artificial Sequence
  ankyrin repeat domain
ggttctgatttaggcaagaagctgttggaggcggcccgtgctggccaggacgacgaagtccgtgagctgctgaaagcgggtgcggacgtgaatgccaaagacgtgttgggctggacccctctgcatctggctgccttcgagggccacctggagattgtggaagtccttctcaaggctggcgctgatgtgaacgcaaaggacaaaaagggttggactccgctgcagctggcggcgcgtaccggtcacctcgaaattgtagaggtcctgttgaaggcgggcgcagatgtaaatgctaaagaccatattggcgcaactccgctccacctggcggcatggcagggccacccagagatcgtggaggtcctcctgaaggcaggcgccgacgtgaacgcccaagacaaatccggtaagactccggcggacctggctgccgacgcaggccatgaggacatcgcggaagtcttgcagaaggcggct

SEQ ID NO: 157 - protein, 28 aa, Artificial Sequence
  C-cap
QDKSGKTPADLAADAGHEDIAEVLQKLN

SEQ ID NO: 158 - protein, 28 aa, Artificial Sequence
  C-cap
QDKSGKTPADLAADAGHEDIAEVLQKAA

SEQ ID NO: 159 - protein, 126 aa, Artificial Sequence
  ankyrin repeat domain
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 160 - protein, 126 aa, Artificial Sequence
  ankyrin repeat domain
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

(19) United States
(12) Patent Application Publication
(10) Pub. No.: US 2022/0242973 A1
(43) Pub. Date: Aug. 4, 2022

(54) RECOMBINANT FAP BINDING PROTEINS AND THEIR USE

(71) Applicant: MOLECULAR PARTNERS AG, Schlieren (CH)

(72) Inventors: Ulrike FIEDLER, Lorrach (DE); Clara METZ, Schlieren (CH); Misha Roland MULLER, Oberkirch (CH); Dan SNELL, Thalwil (CH)

(21) Appl. No.: 17/596,095

(22) PCT Filed: Jun. 3, 2020
(86) PCT No.: PCT/EP2020/065317

§ 371 Date: Dec. 2, 2021

(30) Foreign Application Priority Data
Jun. 4, 2019 (EP) 19178277.0

(51) Int. Cl. C07K 16/40; C07K 14/47; A61K 47/68; A61P 35/00; A61K 51/10

(52) U.S. Cl. CPC C07K 16/40; C07K 14/47; A61K 47/6871; A61P 35/00; A61K 51/1075; A61K 2039/505; C07K 2317/74; C07K 2317/92; C07K 2318/20; C07K 2319/70; C07K 2317/73

(57) ABSTRACT

The present disclosure relates to recombinant binding proteins comprising a designed ankyrin repeat domain with binding specificity for fibroblast activation protein (FAP). In addition, the disclosure relates to nucleic acids encoding such binding proteins, pharmaceutical compositions comprising such binding proteins or nucleic acids, and the use of such binding proteins, nucleic acids or pharmaceutical compositions in methods for localizing or delivering a biologically active molecule in FAP-expressing tissue, such as tumor tissue, and for treating, diagnosing or imaging diseases, such as cancer, in a mammal, including a human.

### CROSS-REFERENCE TO RELATED APPLICATION

The present application claims the benefit of and priority from European patent application EP19178277 filed on 4 Jun. 2019 with the European Patent Office. The content of European patent application EP19178277 is incorporated herein by reference in its entirety, including all tables, figures, and claims.

### FIELD OF THE DISCLOSURE

The present invention relates to recombinant binding proteins comprising a designed ankyrin repeat domain with binding specificity for fibroblast activation protein (FAP). In addition, the invention relates to nucleic acids encoding such binding proteins, pharmaceutical compositions comprising such binding proteins or nucleic acids, and the use of such binding proteins, nucleic acids or pharmaceutical compositions in methods for localizing or delivering a biomolecule or bioactive compound in FAP-expressing tissue, such as tumor tissue, and for treating, diagnosing or imaging diseases, such as cancer, in a mammal, including a human.

### BACKGROUND

Fibroblast-activation protein a (FAP), also known as Seprase, is a type II integral membrane serine peptidase. FAP belongs to the dipeptidyl peptidase IV family (Yu et al., FEBS J. 277, 1126-1144 (2010)). It is a 170 kDa homodimer containing two N-glycosylated subunits with a large C-terminal extracellular domain, in which the enzyme's catalytic domain is located (Scanlan et al., Proc Natl Acad Sci USA 91: 5657-5661 (1994); Wonganu et al., Biochim Biophys Acta 1858(8):1876-82 (2016)). FAP, in its glycosylated form, has both post-prolyl dipeptidyl peptidase and gelatinase activities (Sun et al., Protein Expr Purif 24, 274-281 (2002)). Homologues of human FAP were found in several species, including mice and cynomolgus monkeys (Macaca fascicularis).

FAP is expressed selectively in reactive stromal fibroblasts of more than 90% of epithelial malignancies (primary and metastatic) examined, including lung, colorectal, bladder, ovarian and breast carcinomas, and in malignant mesenchymal cells of bone and soft tissue sarcomas, while it is generally absent from normal adult tissues (Brennen et al., Mol. Cancer Ther. 11(2): 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3114 (1988)). FAP is also expressed on certain malignant tumor cells.

Due to its expression in many common cancers and its restricted expression in normal tissues, FAP has been considered a promising antigenic target for imaging, diagnosis and therapy of a variety of cancers. Various approaches have been devised to exploit the selective expression of FAP in tumor stroma for clinical benefit, including monoclonal antibodies against FAP, small-molecule inhibitors of FAP enzymatic activity, FAP-activated prodrugs of cytotoxic compounds and FAP-specific CAR T cells.

Multiple monoclonal antibodies against FAP have been developed, e.g., Sibrotuzumab, a humanized version of the F19 antibody that specifically binds to human FAP (Scott et al., Clin. Cancer Res. 9, 1639-1647 (2003)); further humanized or fully human antibodies against human FAP with F19 epitope specificity (Mersmann et al., Int J Cancer 92, 240-248 (2001); Schmidt et al., Eur J Biochem 268, 1730-1738 (2001)); and scFv MO36, which recognizes an epitope different from that recognized by F19 and is cross-reactive for the human and mouse FAP proteins (Brocks et al., Mol Med 7, 461-469 (2001). Some of the antibodies against FAP inhibit the enzymatic activity of FAP. For example, the scFv antibody E3 and a derivative thereof significantly inhibited FAP enzymatic activity and biological function (Zhang et al., FASEB J. 2013 February; 27(2): 581-589). The role of FAP in tumor biology is complex and not fully understood, and hence the potential effects of FAP inhibition in tumor biology are also not fully understood.

Specific antibody fusion molecules have further been developed, for example, Bauer et al. (Journal of Immunology 172: 3930-3939 (2004)) reported the generation of a fusion protein consisting of a humanized anti-FAP antibody and human tumor necrosis factor (TNF) replacing the IgG1 CH2/CH3 Fc domain. Furthermore, Brünker et al. (Mol. Cancer Ther. 15(5): 946-57 (2016)) reported the engineering of a bispecific antibody, which simultaneously targets FAP on cancer-associated fibroblasts in tumor stroma and the death receptor DR5 on tumor cells.

Tumor imaging and cancer diagnostic approaches have also been described, for example, Rüger et al. (J. Control Release 186:1-10 (2014)) reported the generation of a fluorescence-activatable liposome, bearing specific single-chain Fv fragments directed against FAP, and evaluated its potential for use in fluorescence diagnostic imaging of FAP-expressing tumor cells by whole body fluorescence imaging. Furthermore, Hua et al. (Diagnostic Pathology 6:111 (2011)) investigated whether staining for FAP with an anti-FAP antibody may be helpful in determining whether ductal carcinoma in situ (DCIS) breast cancer has resulted in microinvasion.

FAP expression in the tumor stroma has also led to attempts to develop locally activated prodrugs. Brennen et al. (Mol. Cancer Ther. 11(2): 257-266 (2012)) discussed an approach of taking advantage of FAP's restricted expression and unique substrate preferences to develop a FAP-activated prodrug to target the activation of a cytotoxic compound within the tumor stroma.

Taken together, it has been suggested that antibodies against FAP can be useful for cancer therapy, imaging and diagnostics. Despite first encouraging results, there still remains a need for therapeutic, diagnostic and imaging approaches for the treatment and characterization of cancers benefitting from FAP-specific binding.

### SUMMARY

The present invention provides recombinant binding proteins comprising a designed ankyrin repeat domain with binding specificity for fibroblast activation protein (FAP). In addition, the invention provides nucleic acids encoding such binding proteins, pharmaceutical compositions comprising such binding proteins or nucleic acids, and the use of such binding proteins, nucleic acids or pharmaceutical compositions in methods for localizing or delivering a biologically active molecule to FAP-expressing cells or tissue, such as tumor tissue, and for treating, diagnosing or imaging diseases, such as cancer, in a mammal, including a human.

In one aspect, the invention provides such a recombinant binding protein comprising an ankyrin repeat domain with binding specificity for FAP, wherein said ankyrin repeat domain comprises an amino acid sequence with at least 75% and up to 100% amino acid sequence identity with any one of the ankyrin repeat domains of SEQ ID NOs: 1 to 35 and 144 to 153, wherein G at position 1 and/or Sat position 2 of said ankyrin repeat domains are optionally missing; and L at the second last position and/or N at the last position of said ankyrin repeat domains of SEQ ID NOs: 1 to 33, 144, 145 and 148 to 150 are optionally exchanged by A. As an example, in one particular embodiment, the FAP-specific recombinant binding protein of the invention comprises the amino acid sequence of SEQ ID NO: 34.

In one aspect, the invention provides such a recombinant binding protein comprising an ankyrin repeat domain with binding specificity for FAP, wherein said ankyrin repeat domain comprises an ankyrin repeat module with at least 80% and up to 100% amino acid sequence identity with any one of the ankyrin repeat modules of SEQ ID NOs: 48 to 134. As an example, in one particular embodiment, the FAP-specific recombinant binding protein of the invention comprises an ankyrin repeat domain with binding specificity for FAP, wherein said ankyrin repeat domain comprises an ankyrin repeat module with an amino acid sequence selected from SEQ ID NOs: 94 to 96. In one particular embodiment, the FAP-specific recombinant binding protein of the invention comprises an ankyrin repeat domain with binding specificity for FAP, wherein said ankyrin repeat domain comprises an ankyrin repeat module with the amino acid sequence of SEQ ID NO: 94, an ankyrin repeat module with the amino acid sequence of SEQ ID NO: 95, and an ankyrin repeat module with the amino acid sequence of SEQ ID NO: 96.

In another aspect, the invention provides such FAP-specific recombinant binding proteins, wherein the binding proteins further comprise a biologically active molecule. The biologically active molecule may be selected from molecules of different structural and functional classes, including, e.g., peptides, polypeptides, toxins, polymers, and nucleic acids. In one aspect of the invention, the biologically active molecule is covalently bound to the FAP-specific recombinant binding protein. The covalent bond may be a peptide bond between the FAP-specific binding protein and a biologically active peptide or polypeptide, resulting in a fusion protein. Alternatively, the biologically active molecule may be covalently conjugated to the FAP-specific binding protein. As an example, in one particular embodiment, a FAP-specific recombinant binding protein of the invention comprises an ankyrin repeat domain with binding specificity for FAP fused to another ankyrin repeat domain with binding specificity for serum albumin. In particular embodiments, such a fusion protein is provided by SEQ ID NOs: 40 to 42. In one particular embodiment, a FAP-specific recombinant binding protein of the invention comprises an ankyrin repeat domain with binding specificity for FAP fused to another polypeptide with binding specificity for a target relevant in cancer biology, such as, e.g., a tumor-associated antigen, an immune-inhibitory molecule, or an immune-stimulatory molecule. In another embodiment, a FAP-specific recombinant binding protein of the invention comprises an ankyrin repeat domain with binding specificity for FAP, fused to another ankyrin repeat domain with binding specificity for serum albumin and also fused to another polypeptide with binding specificity for a target relevant in cancer biology, such as, e.g., a tumor-associated antigen, an immune-inhibitory molecule, or an immune-stimulatory molecule.

In another aspect, the invention provides nucleic acids encoding the FAP-specific binding proteins of the invention and pharmaceutical compositions comprising a FAP-specific binding protein or nucleic acid of the invention and a pharmaceutically acceptable carrier and/or diluent.

In another aspect, the invention provides a method of localizing, accumulating and/or activating a biologically active molecule in a FAP-expressing tissue in a mammal, including a human, the method comprising administering to said mammal a FAP-specific binding protein of the invention comprising the biologically active molecule. In one particular embodiment, such method comprises administering to said mammal a FAP-specific binding protein comprising the biologically active molecule and in addition being covalently linked to or comprising a molecule extending the serum half-life of the binding protein. Such half-life extending molecules are well known in the art and include, for example, polyethylene glycol (PEG), the Fc part of an antibody, and others. In one particular embodiment of the invention, such molecule extending the serum half-life of the binding protein is an ankyrin repeat domain with binding specificity for serum albumin. In one particular embodiment, such method comprises administering the FAP-specific binding protein to a mammal, including a human patient, with a FAP-expressing tumor, resulting in localizing, accumulating and/or activating the biologically active molecule in the FAP-expressing tumor tissue. In one particular embodiment, such FAP-specific binding protein comprises the amino acid sequence of SEQ ID NO: 34 and the amino acid sequence of SEQ ID NO: 38, or of sequence variants of SEQ ID NO: 34 and/or SEQ ID NO: 38 with equivalent target specificities.

In another aspect, the invention provides a method for treating a medical condition in a mammal, including a human patient, the method comprising administering to said mammal a FAP-specific binding protein of the invention covalently linked to or comprising a biologically active molecule, wherein the biologically active molecule is a therapeutically effective molecule. In one particular embodiment, the medical condition is cancer, wherein the cancer or tumor tissue expresses FAP, and the therapeutically effective molecule is an anti-cancer agent. In one particular embodiment, the therapeutically effective molecule is activated only when the FAP-specific binding protein binds to FAP expressed on the surface of cells, such as FAP-expressing fibroblasts in the tumor stroma. In one embodiment, said cancer is selected from colorectal cancers, non-small cell lung cancers, breast cancers, head and neck cancer, ovarian cancers, gastric cancers, lung cancers, invasive bladder cancers, pancreatic cancers, metastatic cancers of the brain, head and neck squamous cell carcinoma, esophagus squamous cell carcinoma, lung squamous cell carcinoma, skin squamous cell carcinoma, melanoma, breast adenocarcinoma, lung adenocarcinoma, cervix squamous cell carcinoma, pancreas squamous cell carcinoma, colon squamous cell carcinoma, or stomach squamous cell carcinoma, prostate cancer, osteosarcoma or soft tissue sarcoma and benign tumors expressing FAP. In one embodiment, such cancer is selected from epithelial malignancies (primary and metastatic), including lung, colorectal, bladder, gastric, prostate, ovarian and breast carcinomas, and bone and soft tissue sarcomas.

The invention further provides a kit comprising the recombinant binding protein of the invention, a nucleic acid of the invention or a pharmaceutical composition of the invention. The invention further provides a method for producing the recombinant binding protein of the invention, the method comprising the steps of (i) expressing said recombinant binding protein in bacteria, and (ii) purifying said recombinant binding protein using chromatography.

### BRIEF DESCRIPTION OF THE FIGURES

FIG. 1A: SDS-PAGE gel analysis of the purification of a selected ankyrin repeat protein with binding specificity for human FAP, DARPin® protein #18. M corresponds to a protein size marker.

FIG. 1B: SDS-PAGE gel analysis of the purification of another selected ankyrin repeat protein with binding specificity for human FAP, DARPin® protein #34. M corresponds to a protein size marker.

FIG. 2: Surface Plasmon Resonance (SPR) analysis of ankyrin repeat protein binding to human FAP, exemplified by DARPin® protein #34. Various concentrations (0.4, 1.1, 3.3, and 10 nM) of purified ankyrin repeat protein were applied to a GLC chip with immobilized human FAP for on-rate and off-rate measurements. The obtained SPR trace analyses were used to determine the ankyrin repeat protein—FAP interaction. RU, Resonance Units; s, time in seconds.

FIG. 3: Binding of FAP-specific ankyrin repeat proteins to FAP+ cells (WI38 cells), as exemplified by DARPin® protein #18, DARPin® protein #19, DARPin® protein #26 and DARPin® protein #33. Concentration-dependent binding curves of are shown in relation to the determined median fluorescence intensity (MFI).

FIG. 4A: Binding of FAP-specific DARPin® protein #34 and DARPin® protein #35 to FAP+ cells (WI38 cells). Concentration-dependent binding curves of DARPin® protein #34 and DARPin® protein #35 are shown in relation to the calculated fold increase of MFI.

FIG. 4B: Binding of FAP-specific DARPin® protein #34 and DARPin® protein #35 to CHO-wt cells and CHO-FAP1.9 cells. Concentration-dependent binding curves of DARPin® protein #34 and DARPin® protein #35 are shown in relation to the calculated fold increase of MFI. DARPin® protein #34 and DARPin® protein #35 bind only to CHO-FAP1.9 cells expressing FAP on the cell surface.

FIG. 5A: Surface Plasmon Resonance (SPR) analysis of ankyrin repeat protein binding to human FAP, exemplified by DARPin® protein #36. Various concentrations (3.13, 6.25, 12.5, and 25 nM) of ankyrin repeat protein were applied to a GLC chip with immobilized human FAP for on-rate and off-rate measurements. The obtained SPR trace analyses were used to determine the ankyrin repeat protein—FAP interaction. RU, Resonance Units; s, time in seconds.

FIG. 5B: Surface Plasmon Resonance (SPR) analysis of ankyrin repeat protein binding to cynomolgus FAP (cFAP), exemplified by DARPin® protein #36. Various concentrations (3.13, 6.25, 12.5, and 25 nM) of ankyrin repeat protein were applied to a GLC chip with immobilized cynomolgus FAP for on-rate and off-rate measurements. The obtained SPR trace analyses were used to determine the ankyrin repeat protein—FAP interaction. RU, Resonance Units; s, time in seconds.

FIG. 6: Binding of FAP-specific ankyrin repeat proteins to cynomolgus FAP+CHO cells, as exemplified by DARPin® protein #18, DARPin® protein #19, DARPin® protein #26 and DARPin® protein #33. Concentration-dependent binding curves are shown in relation to the determined median fluorescence intensity (MFI).

FIG. 7A: Binding of FAP-specific DARPin® protein #40 and DARPin® protein #41 to U87MG cells. Concentration-dependent binding curves of DARPin® protein #40 and DARPin® protein #41 are shown for in relation to the determined median fluorescence intensity (MFI).

FIG. 7B: Binding of FAP-specific DARPin® protein #40 and DARPin® protein #41 to WI38 cells. Concentration-dependent binding curves of DARPin® protein #40 and DARPin® protein #41 are shown in relation to the determined median fluorescence intensity (MFI).

FIG. 8: Serum concentrations of a binding protein comprising a FAP-specific ankyrin repeat domain fused to a biologically active molecule (DARPin® protein #40, DARPin® protein #41 and DARPin® protein #42) as a function of time after a single intravenous bolus injection into the tail vein of mice.

FIG. 9: Biodistribution of DARPin® protein #41 and control compound DARPin® protein #43, both labeled with Tc99^{m}, assessed in a FAP-positive mouse tumor model at 48 hours post-injection and determined as organ/blood ratios of the measured radioactivity.

### DETAILED DESCRIPTION

As disclosed and exemplified herein, the disclosure provides ankyrin repeat proteins that specifically target FAP. Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

DARPin® is a trademark owned by Molecular Partners AG, Switzerland.

In one aspect, the invention relates to a recombinant binding protein comprising an ankyrin repeat domain, wherein said ankyrin repeat domain has binding specificity for fibroblast activation protein (FAP), and wherein said ankyrin repeat domain comprises an ankyrin repeat module comprising an amino acid sequence selected from the group consisting of (1) SEQ ID NOs: 48 to 134 and (2) sequences in which up to 9, or up to 8, or up to 7, or up to 6, or up to 5, or up to 4, or up to 3, or up to 2, or up to 1 amino acids in any of SEQ ID NOs: 48 to 134 are exchanged by another amino acid. Thus, in one embodiment, said ankyrin repeat module comprises an amino acid sequence selected from the group consisting of (1) SEQ ID NOs: 48 to 134 and (2) sequences in which up to 3 amino acids in any of SEQ ID NOs: 48 to 134 are exchanged by another amino acid. In one embodiment, said ankyrin repeat module comprises an amino acid sequence selected from the group consisting of (1) SEQ ID NOs: 48 to 134 and (2) sequences in which up to 2 amino acids in any of SEQ ID NOs: 48 to 134 are exchanged by another amino acid. In one embodiment, said ankyrin repeat module comprises an amino acid sequence selected from the group consisting of (1) SEQ ID NOs: 48 to 134 and (2) sequences in which up to 1 amino acid in any of SEQ ID NOs: 48 to 134 is exchanged by another amino acid. In one embodiment, all of said 9, 8, 7, 6, 5, 4, 3, 2, or 1 amino acid exchanges occur in framework positions of said ankyrin repeat module(s), wherein typically the overall structure of the module(s) is not affected by the exchanges. In one embodiment, all of said 9, 8, 7, 6, 5, 4, 3, 2, or 1 amino acid exchanges occur in positions other than the randomized positions 3, 4, 6, 14 and 15 of said ankyrin repeat module(s). In one embodiment, said ankyrin repeat module comprises an amino acid sequence selected from the group consisting of SEQ ID NOs: 48 to 134.

In one embodiment, said ankyrin repeat module comprises an amino acid sequence selected from the group consisting of (1) SEQ ID NOs: 94 to 98, 111 to 113 and 132 to 134 and (2) sequences in which up to 9, or up to 8, or up to 7, or up to 6, or up to 5, or up to 4, or up to 3, or up to 2, or up to 1 amino acids in any of SEQ ID NOs: SEQ ID NOs: 94 to 98, 111 to 113 and 132 to 134 are exchanged by another amino acid. Thus, in one embodiment, said ankyrin repeat module comprises an amino acid sequence selected from the group consisting of (1) SEQ ID NOs: 94 to 98, 111 to 113 and 132 to 134 and (2) sequences in which up to 3 amino acids in any of SEQ ID NOs: SEQ ID NOs: 94 to 98, 111 to 113 and 132 to 134 are exchanged by another amino acid. In one embodiment, said ankyrin repeat module comprises an amino acid sequence selected from the group consisting of (1) SEQ ID NOs: 94 to 98, 111 to 113 and 132 to 134 and (2) sequences in which up to 2 amino acids in any of SEQ ID NOs: SEQ ID NOs: 94 to 98, 111 to 113 and 132 to 134 are exchanged by another amino acid. In one embodiment, said ankyrin repeat module comprises an amino acid sequence selected from the group consisting of (1) SEQ ID NOs: 94 to 98, 111 to 113 and 132 to 134 and (2) sequences in which up to 1 amino acid in any of SEQ ID NOs: SEQ ID NOs: 94 to 98, 111 to 113 and 132 to 134 is exchanged by another amino acid. In one embodiment, all of said 9, 8, 7, 6, 5, 4, 3, 2, or 1 amino acid exchanges occur in framework positions of said ankyrin repeat module(s), wherein typically the overall structure of the module(s) is not affected by the exchanges. In one embodiment, all of said 9, 8, 7, 6, 5, 4, 3, 2, or 1 amino acid exchanges occur in positions other than the randomized positions 3, 4, 6, 14 and 15 of said ankyrin repeat module(s). In one embodiment, said ankyrin repeat module comprises an amino acid sequence selected from the group consisting of SEQ ID NOs: 94 to 98, 111 to 113 and 132 to 134.

In one embodiment, said ankyrin repeat module comprises the amino acid sequence of SEQ ID NO: 94 or a sequence in which one or two amino acids in SEQ ID NO: 94 are exchanged by another amino acid. In one embodiment, said ankyrin repeat module comprises the amino acid sequence of SEQ ID NO: 95 or a sequence in which one or two amino acids in SEQ ID NO: 95 are exchanged by another amino acid. In one embodiment, said ankyrin repeat module comprises the amino acid sequence of SEQ ID NO: 96 or a sequence in which one or two amino acids in SEQ ID NO: 96 are exchanged by another amino acid. In one embodiment, said ankyrin repeat module comprises the amino acid sequence of SEQ ID NO: 97 or a sequence in which one or two amino acids in SEQ ID NO: 97 are exchanged by another amino acid. In one embodiment, said ankyrin repeat module comprises the amino acid sequence of SEQ ID NO: 98 or a sequence in which one or two amino acids in SEQ ID NO: 98 are exchanged by another amino acid. In one embodiment, said ankyrin repeat module comprises the amino acid sequence of SEQ ID NO: 94. In one embodiment, said ankyrin repeat module comprises the amino acid sequence of SEQ ID NO: 95. In one embodiment, said ankyrin repeat module comprises the amino acid sequence of SEQ ID NO: 96. In one embodiment, said ankyrin repeat module comprises the amino acid sequence of SEQ ID NO: 97. In one embodiment, said ankyrin repeat module comprises the amino acid sequence of SEQ ID NO: 98.

In one embodiment, all of said amino acid exchanges of said ankyrin repeat module(s) as described and referred to herein occur in framework positions of said ankyrin repeat module(s), wherein typically the overall structure of the module(s) is not affected by the exchanges. Such an embodiment of exchange in framework positions shall apply to all embodiments irrespective of whether such exchange is explicitly described. In one embodiment, all of said amino acid exchanges of said ankyrin repeat module(s) as described and referred to herein occur in positions other than the randomized positions 3, 4, 6, 14 and 15 of said ankyrin repeat module(s). Such an embodiment of exchange in positions other than the randomized positions 3, 4, 6, 14 and 15 shall apply to all embodiments irrespective of whether such exchange is explicitly described.

In one embodiment, said ankyrin repeat domain comprises a first ankyrin repeat module and a second ankyrin repeat module. In one embodiment, said first ankyrin repeat module is located N-terminally of said second ankyrin repeat module within said ankyrin repeat domain.

In one embodiment, said ankyrin repeat domain comprises a first ankyrin repeat module and a second ankyrin repeat module and a third ankyrin repeat module. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one embodiment, said first, said second and, if present, said third ankyrin repeat module comprises an amino acid sequence selected from the group consisting of (1) SEQ ID NOs: 48 to 134 and (2) sequences in which up to 9, or up to 8, or up to 7, or up to 6, or up to 5, or up to 4, or up to 3, or up to 2, or up to 1 amino acids in any of SEQ ID NOs: 48 to 134 are exchanged by another amino acid. In one embodiment, said first, said second and, if present, said third ankyrin repeat module comprises an amino acid sequence selected from the group consisting of (1) SEQ ID NOs: 94 to 98, 111 to 113 and 132 to 134 and (2) sequences in which up to 9, or up to 8, or up to 7, or up to 6, or up to 5, or up to 4, or up to 3, or up to 2, or up to 1 amino acids in any of SEQ ID NOs: SEQ ID NOs: 94 to 98, 111 to 113 and 132 to 134 are exchanged by another amino acid.

In one embodiment, said ankyrin repeat domain comprises a first ankyrin repeat module and a second ankyrin repeat module and a third ankyrin repeat module. In one embodiment, in such an ankyrin repeat domain said first ankyrin repeat module comprises an amino acid sequence selected from the group consisting of (1) SEQ ID NO: 94 and (2) sequences in which up to 9, or up to 8, or up to 7, or up to 6, or up to 5, or up to 4, or up to 3, or up to 2, or up to 1 amino acids in SEQ ID NO: 94 are exchanged by another amino acid, and said second ankyrin repeat module comprises an amino acid sequence selected from the group consisting of (1) SEQ ID NO: 95 and (2) sequences in which up to 9, or up to 8, or up to 7, or up to 6, or up to 5, or up to 4, or up to 3, or up to 2, or up to 1 amino acids of SEQ ID NO: 95 are exchanged by another amino acid, and said third ankyrin repeat module comprises an amino acid sequence selected from the group consisting of (1) SEQ ID NO: 96 and (2) sequences in which up to 9, or up to 8, or up to 7, or up to 6, or up to 5, or up to 4, or up to 3, or up to 2, or up to 1 amino acids of SEQ ID NO: 96 are exchanged by another amino acid. In one embodiment, all of said 9, 8, 7, 6, 5, 4, 3, 2, or 1 amino acid exchanges occur in framework positions of said ankyrin repeat module(s), wherein typically the overall structure of the module(s) is not affected by the exchanges. In one embodiment, all of said 9, 8, 7, 6, 5, 4, 3, 2, or 1 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which up to 6 amino acids in SEQ ID NO: 94 are exchanged by another amino acid, and said second ankyrin repeat module comprises an amino acid sequence selected from the group consisting of (1) SEQ ID NO: 95 and (2) sequences in which up to 6 amino acids of SEQ ID NO: 95 are exchanged by another amino acid, and said third ankyrin repeat module comprises an amino acid sequence selected from the group consisting of (1) SEQ ID NO: 96 and (2) sequences in which up to 6 amino acids of SEQ ID NO: 96 are exchanged by another amino acid. In one embodiment, all of said 6 amino acid exchanges occur in framework positions of said ankyrin repeat module(s), wherein typically the overall structure of the module(s) is not affected by the exchanges. In one embodiment, all of said 6 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which up to 5 amino acids in SEQ ID NO: 94 are exchanged by another amino acid, and said second ankyrin repeat module comprises an amino acid sequence selected from the group consisting of (1) SEQ ID NO: 95 and (2) sequences in which up to 5 amino acids of SEQ ID NO: 95 are exchanged by another amino acid, and said third ankyrin repeat module comprises an amino acid sequence selected from the group consisting of (1) SEQ ID NO: 96 and (2) sequences in which up to 5 amino acids of SEQ ID NO: 96 are exchanged by another amino acid. In one embodiment, all of said 5 amino acid exchanges occur in framework positions of said ankyrin repeat module(s), wherein typically the overall structure of the module(s) is not affected by the exchanges. In one embodiment, all of said 5 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which up to 4 amino acids in SEQ ID NO: 94 are exchanged by another amino acid, and said second ankyrin repeat module comprises an amino acid sequence selected from the group consisting of (1) SEQ ID NO: 95 and (2) sequences in which up to 4 amino acids of SEQ ID NO: 95 are exchanged by another amino acid, and said third ankyrin repeat module comprises an amino acid sequence selected from the group consisting of (1) SEQ ID NO: 96 and (2) sequences in which up to 4 amino acids of SEQ ID NO: 96 are exchanged by another amino acid. In one embodiment, all of said 4 amino acid exchanges occur in framework positions of said ankyrin repeat module(s), wherein typically the overall structure of the module(s) is not affected by the exchanges. In one embodiment, all of said 4 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which up to 3 amino acids in SEQ ID NO: 94 are exchanged by another amino acid, and said second ankyrin repeat module comprises an amino acid sequence selected from the group consisting of (1) SEQ ID NO: 95 and (2) sequences in which up to 3 amino acids of SEQ ID NO: 95 are exchanged by another amino acid, and said third ankyrin repeat module comprises an amino acid sequence selected from the group consisting of (1) SEQ ID NO: 96 and (2) sequences in which up to 3 amino acids of SEQ ID NO: 96 are exchanged by another amino acid. In one embodiment, all of said 3 amino acid exchanges occur in framework positions of said ankyrin repeat module(s), wherein typically the overall structure of the module(s) is not affected by the exchanges. In one embodiment, all of said 3 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which up to 2 amino acids in SEQ ID NO: 94 are exchanged by another amino acid, and said second ankyrin repeat module comprises an amino acid sequence selected from the group consisting of (1) SEQ ID NO: 95 and (2) sequences in which up to 2 amino acids of SEQ ID NO: 95 are exchanged by another amino acid, and said third ankyrin repeat module comprises an amino acid sequence selected from the group consisting of (1) SEQ ID NO: 96 and (2) sequences in which up to 2 amino acids of SEQ ID NO: 96 are exchanged by another amino acid. In one embodiment, all of said 2 amino acid exchanges occur in framework positions of said ankyrin repeat module(s), wherein typically the overall structure of the module(s) is not affected by the exchanges. In one embodiment, all of said 2 amino acid exchanges occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 94 and (2) sequences in which 1 amino acid in SEQ ID NO: 94 is exchanged by another amino acid, and said second ankyrin repeat module comprises an amino acid sequence selected from the group consisting of (1) SEQ ID NO: 95 and (2) sequences in which 1 amino acid of SEQ ID NO: 95 is exchanged by another amino acid, and said third ankyrin repeat module comprises an amino acid sequence selected from the group consisting of (1) SEQ ID NO: 96 and (2) sequences in which 1 amino acid of SEQ ID NO: 96 is exchanged by another amino acid. In one embodiment, all of said amino acid exchanges occur in framework positions of said ankyrin repeat module(s), wherein typically the overall structure of the module(s) is not affected by the exchanges. In one embodiment, all of said amino acid exchanges occur in positions other than the randomized positions 3, 4, 6, 14 and 15 of said ankyrin repeat module(s).

In one embodiment, in such an ankyrin repeat domain said first ankyrin repeat module comprises the amino acid sequence of SEQ ID NO: 94, and said second ankyrin repeat module comprises the amino acid sequence of SEQ ID NO: 95, and said third ankyrin repeat module comprises the amino acid sequence of SEQ ID NO: 96.

In one embodiment, said first ankyrin repeat module is located N-terminally of said second ankyrin repeat module within said ankyrin repeat domain, and wherein said second ankyrin repeat module is located N-terminally of said third ankyrin repeat module within said ankyrin repeat domain. Thus, in one embodiment, said ankyrin repeat module is a first ankyrin repeat module and comprises an amino acid sequence selected from the group consisting of (1) SEQ ID NO: 94 and (2) sequences in which up to 3, or up to 2, or up to 1 amino acids in SEQ ID NO: 94 are exchanged by another amino acid, and wherein said ankyrin repeat domain further comprises a second ankyrin repeat module comprising an amino acid sequence selected from the group consisting of (1) SEQ ID NO: 95 and (2) sequences in which up to 3, or up to 2, or up to 1 amino acids of SEQ ID NO: 95 are exchanged by another amino acid, and wherein said ankyrin repeat domain further comprises a third ankyrin repeat module comprising an amino acid sequence selected from the group consisting of (1) SEQ ID NO: 96 and (2) sequences in which up to 3, or up to 2, or up to 1 amino acids of SEQ ID NO: 96 are exchanged by another amino acid, and wherein said first ankyrin repeat module is located N-terminally of said second ankyrin repeat module within said ankyrin repeat domain, and wherein said second ankyrin repeat module is located N-terminally of said third ankyrin repeat module within said ankyrin repeat domain. In one embodiment, all of said up to 3, or up to 2, or up to 1 amino acid exchanges occur in framework positions of said ankyrin repeat module(s), wherein typically the overall structure of the module(s) is not affected by the exchanges. In one embodiment, all of said up to 3, or up to 2, or up to 1 amino acid exchanges occur in positions other than the randomized positions 3, 4, 6, 14 and 15 of said ankyrin repeat module(s). Further, in one embodiment, said ankyrin repeat domain comprises a first, a second, and a third ankyrin repeat module, wherein said first ankyrin repeat module comprises the amino acid sequence of SEQ ID NO: 94, and wherein said second ankyrin repeat module comprises the amino acid sequence of SEQ ID NO: 95, and wherein said third ankyrin repeat module comprises the amino acid sequence of SEQ ID NO: 96, and wherein said first ankyrin repeat module is located N-terminally of said second ankyrin repeat module within said ankyrin repeat domain, and wherein said second ankyrin repeat module is located N-terminally of said third ankyrin repeat module within said ankyrin repeat domain.

In one embodiment, said ankyrin repeat domain comprises a first ankyrin repeat module and a second ankyrin repeat module. In one embodiment, in such an ankyrin repeat domain said first ankyrin repeat module comprises an amino acid sequence selected from the group consisting of (1) SEQ ID NO: 97 and (2) sequences in which up to 9, or up to 8, or up to 7, or up to 6, or up to 5, or up to 4, or up to 3, or up to 2, or up to 1 amino acids in SEQ ID NO: 97 are exchanged by another amino acid, and said second ankyrin repeat module comprises an amino acid sequence selected from the group consisting of (1) SEQ ID NO: 98 and (2) sequences in which up to 9, or up to 8, or up to 7, or up to 6, or up to 5, or up to 4, or up to 3, or up to 2, or up to 1 amino acids of SEQ ID NO: 98 are exchanged by another amino acid. In one embodiment, in such an ankyrin repeat domain said first ankyrin repeat module comprises an amino acid sequence selected from the group consisting of (1) SEQ ID NO: 97 and (2) sequences in which up to 6 amino acids in SEQ ID NO: 97 are exchanged by another amino acid, and said ankyrin second ankyrin repeat module comprises an amino acid sequence selected from the group consisting of (1) SEQ ID NO: 98 and (2) sequences in which up to 6 amino acids of SEQ ID NO: 98 are exchanged by another amino acid. In one embodiment, in such an ankyrin repeat domain said first ankyrin repeat module comprises an amino acid sequence selected from the group consisting of (1) SEQ ID NO: 97 and (2) sequences in which up to 5 amino acids in SEQ ID NO: 97 are exchanged by another amino acid, and said second ankyrin repeat module comprises an amino acid sequence selected from the group consisting of (1) SEQ ID NO: 98 and (2) sequences in which up to 5 amino acids of SEQ ID NO: 98 are exchanged by another amino acid. In one embodiment, in such an ankyrin repeat domain said first ankyrin repeat module comprises an amino acid sequence selected from the group consisting of (1) SEQ ID NO: 97 and (2) sequences in which up to 4 amino acids in SEQ ID NO: 97 are exchanged by another amino acid, and said second ankyrin repeat module comprises an amino acid sequence selected from the group consisting of (1) SEQ ID NO: 98 and (2) sequences in which up to 4 amino acids of SEQ ID NO: 98 are exchanged by another amino acid. In one embodiment, in such an ankyrin repeat domain said ankyrin repeat module comprises an amino acid sequence selected from the group consisting of (1) SEQ ID NO: 97 and (2) sequences in which up to 3 amino acids in SEQ ID NO: 97 are exchanged by another amino acid, and said second ankyrin repeat module comprises an amino acid sequence selected from the group consisting of (1) SEQ ID NO: 98 and (2) sequences in which up to 3 amino acids of SEQ ID NO: 98 are exchanged by another amino acid. In one embodiment, in such an ankyrin repeat domain said first ankyrin repeat module comprises an amino acid sequence selected from the group consisting of (1) SEQ ID NO: 97 and (2) sequences in which up to 2 amino acids in SEQ ID NO: 97 are exchanged by another amino acid, and said second ankyrin repeat module comprises an amino acid sequence selected from the group consisting of (1) SEQ ID NO: 98 and (2) sequences in which up to 2 amino acids of SEQ ID NO: 98 are exchanged by another amino acid. In one embodiment, in such an ankyrin repeat domain said first ankyrin repeat module comprises an amino acid sequence selected from the group consisting of (1) SEQ ID NO: 97 and (2) sequences in which 1 amino acid in SEQ ID NO: 97 is exchanged by another amino acid, and said second ankyrin repeat module comprises an amino acid sequence selected from the group consisting of (1) SEQ ID NO: 98 and (2) sequences in which 1 amino acid of SEQ ID NO: 98 is exchanged by another amino acid. In one embodiment, all of said amino acid exchanges occur in framework positions of said ankyrin repeat module(s), wherein typically the overall structure of the module(s) is not affected by the exchanges. In one embodiment, all of said amino acid exchanges occur in positions other than the randomized positions 3, 4, 6, 14 and 15 of said ankyrin repeat module(s). In one embodiment, in such an ankyrin repeat domain said first ankyrin repeat module comprises the amino acid sequence of SEQ ID NO: 97, and said second ankyrin repeat module comprises the amino acid sequence of SEQ ID NO: 98.

In one embodiment, said first ankyrin repeat module is located N-terminally of said second ankyrin repeat module within said ankyrin repeat domain. Thus, in one embodiment, said ankyrin repeat domain comprises a first and a second ankyrin repeat module, wherein said first ankyrin repeat module comprises an amino acid sequence selected from the group consisting of (1) SEQ ID NO: 97 and (2) sequences in which up to 3, or up to 2, or up to 1 amino acids in SEQ ID NO: 97 are exchanged by another amino acid, and said second ankyrin repeat module comprises an amino acid sequence selected from the group consisting of (1) SEQ ID NO: 98 and (2) sequences in which up to 3, or up to 2, or up to 1 amino acids of SEQ ID NO: 98 are exchanged by another amino acid, wherein said first ankyrin repeat module is located N-terminally of said second ankyrin repeat module within said ankyrin repeat domain. In one embodiment, all of said up to 3, or up to 2, or up to 1 amino acid exchanges occur in framework positions of said ankyrin repeat module(s), wherein typically the overall structure of the module(s) is not affected by the exchanges. In one embodiment, all of said up to 3, or up to 2, or up to 1 amino acid exchanges occur in positions other than the randomized positions 3, 4, 6, 14 and 15 of said ankyrin repeat module(s). Further, in one embodiment, said ankyrin repeat domain comprises a first and a second ankyrin repeat module, wherein said first ankyrin repeat module comprises the amino acid sequence of SEQ ID NO: 97, and wherein said second ankyrin repeat module comprises the amino acid sequence of SEQ ID NO: 98, and wherein said first ankyrin repeat module is located N-terminally of said second ankyrin repeat module within said ankyrin repeat domain.

In one embodiment, all of said amino acid exchanges in said ankyrin repeat module(s) described above occur in framework positions and in positions other than the randomized positions 3, 4, 6, 14 and 15 of said ankyrin repeat module(s), wherein typically the overall structure of the module(s) is not affected by the exchanges.

In another aspect, the invention relates to a recombinant binding protein comprising an ankyrin repeat domain, wherein said ankyrin repeat domain has binding specificity for fibroblast activation protein (FAP), and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 1 to 35 and 144 to 153. In another embodiment, said ankyrin repeat domain comprises an amino acid sequence with at least 93% amino acid sequence identity with any one of SEQ ID NOs: 1 to 35 and 144 to 153; and in a further embodiment, said ankyrin repeat domain comprises an amino acid sequence with at least 95% amino acid sequence identity with any one of SEQ ID NOs: 1 to 35 and 144 to 153. In one embodiment, said ankyrin repeat domain comprises an amino acid sequence with at least 98% amino acid sequence identity with any one of SEQ ID NOs: 1 to 35 and 144 to 153; and in one embodiment, said ankyrin repeat domain comprises an amino acid sequence of any one of SEQ ID NOs: 1 to 35 and 144 to 153.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 18, 19, 26, 33, 34, 35 and 144 to 153, wherein G at position 1 and/or S at position 2 of SEQ ID NOs: 18, 19, 26, 33, 34, 35 and 144 to 153 are optionally missing, and wherein L at the second last position and/or N at the last position of SEQ ID NOs: 18, 19, 26, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 18, 19, 26, 33, 34, 35 and 144 to 153. In another embodiment, said ankyrin repeat domain comprises an amino acid sequence with at least 93% amino acid sequence identity with any one of SEQ ID NOs: 18, 19, 26, 33, 34, 35 and 144 to 153; and in a further embodiment, said ankyrin repeat domain comprises an amino acid sequence with at least 95% amino acid sequence identity with any one of SEQ ID NOs: 18, 19, 26, 33, 34, 35 and 144 to 153. In one embodiment, said ankyrin repeat domain comprises an amino acid sequence with at least 98% amino acid sequence identity with any one of SEQ ID NOs: 18, 19, 26, 33, 34, 35 and 144 to 153; and in one embodiment, said ankyrin repeat domain comprises an amino acid sequence of any one of SEQ ID NOs: 18, 19, 26, 33, 34, 35 and 144 to 153. Thus, in one embodiment, said recombinant binding protein comprises an ankyrin repeat domain having binding specificity for FAP, wherein said ankyrin repeat domain comprises an amino acid sequence selected from SEQ ID NOs: 18, 19, 26, 33, 34, 35 and 144 to 153, wherein G at position 1 and/or S at position 2 of SEQ ID NOs: 18, 19, 26, 33, 34, 35 and 144 to 153 are optionally missing, and wherein L at the second last position and/or N at the last position of SEQ ID NOs: 18, 19, 26, 33, 144, 145 and 148 to 150 are optionally exchanged by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 18, 19, 26, 33, 34 and 35, wherein G at position 1 and/or S at position 2 of SEQ ID NOs: 18, 19, 26, 33, 34 and 35 are optionally missing, and wherein L at the second last position and/or N at the last position of SEQ ID NOs: 18, 19, 26 and 33 are optionally exchanged by A. Thus, in one embodiment, said ankyrin repeat domain comprises an amino acid sequence with at least 90% amino acid sequence identity with any one of SEQ ID NOs: 18, 19, 26, 33, 34 and 35. In another embodiment, said ankyrin repeat domain comprises an amino acid sequence with at least 93% amino acid sequence identity with any one of SEQ ID NOs: 18, 19, 26, 33, 34 and 35; and in a further embodiment, said ankyrin repeat domain comprises an amino acid sequence with at least 95% amino acid sequence identity with any one of SEQ ID NOs: 18, 19, 26, 33, 34 and 35. In one embodiment, said ankyrin repeat domain comprises an amino acid sequence with at least 98% amino acid sequence identity with any one of SEQ ID NOs: 18, 19, 26, 33, 34 and 35; and in one embodiment, said ankyrin repeat domain comprises an amino acid sequence of any one of SEQ ID NOs: 18, 19, 26, 33, 34 and 35. Thus, in one embodiment, said recombinant binding protein comprises an ankyrin repeat domain having binding specificity for FAP, wherein said ankyrin repeat domain comprises an amino acid sequence selected from SEQ ID NOs: 18, 19, 26, 33, 34 and 35, wherein G at position 1 and/or S at position 2 of SEQ ID NOs: 18, 19, 26, 33, 34 and 35 are optionally missing, and wherein L at the second last position and/or N at the last position of SEQ ID NOs: 18, 19, 26 and 33 are optionally exchanged by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A. Thus, in one embodiment, said ankyrin repeat domain comprises an amino acid sequence with at least 90% amino acid sequence identity with SEQ ID NO: 18. In another embodiment, said ankyrin repeat domain comprises an amino acid sequence with at least 93% amino acid sequence identity with SEQ ID NO: 18; and in a further embodiment, said ankyrin repeat domain comprises an amino acid sequence with at least 95% amino acid sequence identity with SEQ ID NO: 18. In one embodiment, said ankyrin repeat domain comprises an amino acid sequence with at least 98% amino acid sequence identity with SEQ ID NO: 18; and in one embodiment, said ankyrin repeat domain comprises the amino acid sequence of SEQ ID NO: 18. Thus, in one embodiment, said recombinant binding protein comprises an ankyrin repeat domain having binding specificity for FAP, wherein said ankyrin repeat domain comprises the amino acid sequence of SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 19, wherein G at position 1 and/or S at position 2 of SEQ ID NO: 19 are optionally missing, and wherein L at the second last position and/or N at the last position of SEQ ID NO: 19 are optionally exchanged by A. Thus, in one embodiment, said ankyrin repeat domain comprises an amino acid sequence with at least 90% amino acid sequence identity with SEQ ID NO: 19. In another embodiment, said ankyrin repeat domain comprises an amino acid sequence with at least 93% amino acid sequence identity with SEQ ID NO: 19; and in a further embodiment, said ankyrin repeat domain comprises an amino acid sequence with at least 95% amino acid sequence identity with SEQ ID NO: 19. In one embodiment, said ankyrin repeat domain comprises an amino acid sequence with at least 98% amino acid sequence identity with SEQ ID NO: 19; and in one embodiment, said ankyrin repeat domain comprises the amino acid sequence of SEQ ID NO: 19. Thus, in one embodiment, said recombinant binding protein comprises an ankyrin repeat domain having binding specificity for FAP, wherein said ankyrin repeat domain comprises the amino acid sequence of SEQ ID NO: 19, wherein G at position 1 and/or S at position 2 of SEQ ID NO: 19 are optionally missing, and wherein L at the second last position and/or N at the last position of SEQ ID NO: 19 are optionally exchanged by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 26, wherein G at position 1 and/or S at position 2 of SEQ ID NO: 26 are optionally missing, and wherein L at the second last position and/or N at the last position of SEQ ID NO: 26 are optionally exchanged by A. Thus, in one embodiment, said ankyrin repeat domain comprises an amino acid sequence with at least 90% amino acid sequence identity with SEQ ID NO: 26. In another embodiment, said ankyrin repeat domain comprises an amino acid sequence with at least 93% amino acid sequence identity with SEQ ID NO: 26; and in a further embodiment, said ankyrin repeat domain comprises an amino acid sequence with at least 95% amino acid sequence identity with SEQ ID NO: 26. In one embodiment, said ankyrin repeat domain comprises an amino acid sequence with at least 98% amino acid sequence identity with SEQ ID NO: 26; and in one embodiment, said ankyrin repeat domain comprises the amino acid sequence of SEQ ID NO: 26.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 33, wherein G at position 1 and/or S at position 2 of SEQ ID NO: 33 are optionally missing, and wherein L at the second last position and/or N at the last position of SEQ ID NO: 33 are optionally exchanged by A. Thus, in one embodiment, said ankyrin repeat domain comprises an amino acid sequence with at least 90% amino acid sequence identity with SEQ ID NO: 33. In another embodiment, said ankyrin repeat domain comprises an amino acid sequence with at least 93% amino acid sequence identity with SEQ ID NO: 33; and in a further embodiment, said ankyrin repeat domain comprises an amino acid sequence with at least 95% amino acid sequence identity with SEQ ID NO: 33. In one embodiment, said ankyrin repeat domain comprises an amino acid sequence with at least 98% amino acid sequence identity with SEQ ID NO: 33; and in one embodiment, said ankyrin repeat domain comprises the amino acid sequence of SEQ ID NO: 33.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 34, wherein G at position 1 and/or S at position 2 of SEQ ID NO: 34 are optionally missing. Thus, in one embodiment, said ankyrin repeat domain comprises an amino acid sequence with at least 90% amino acid sequence identity with SEQ ID NO: 34. In another embodiment, said ankyrin repeat domain comprises an amino acid sequence with at least 93% amino acid sequence identity with SEQ ID NO: 34; and in a further embodiment, said ankyrin repeat domain comprises an amino acid sequence with at least 95% amino acid sequence identity with SEQ ID NO: 34. In one embodiment, said ankyrin repeat domain comprises an amino acid sequence with at least 98% amino acid sequence identity with SEQ ID NO: 34; and in one embodiment, said ankyrin repeat domain comprises the amino acid sequence of SEQ ID NO: 34.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 35, wherein G at position 1 and/or S at position 2 of SEQ ID NO: 35 are optionally missing. Thus, in one embodiment, said ankyrin repeat domain comprises an amino acid sequence with at least 90% amino acid sequence identity with SEQ ID NO: 35. In another embodiment, said ankyrin repeat domain comprises an amino acid sequence with at least 93% amino acid sequence identity with SEQ ID NO: 35; and in a further embodiment, said ankyrin repeat domain comprises an amino acid sequence with at least 95% amino acid sequence identity with SEQ ID NO: 35. In one embodiment, said ankyrin repeat domain comprises an amino acid sequence with at least 98% amino acid sequence identity with SEQ ID NO: 35; and in one embodiment, said ankyrin repeat domain comprises the amino acid sequence of SEQ ID NO: 35.

In one embodiment, said recombinant binding protein comprises an ankyrin repeat domain having binding specificity for FAP, wherein the potential interaction residues in said ankyrin repeat domain are identical to the corresponding positions in any one of the ankyrin repeat domains of SEQ ID NOs: 1 to 35.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 144 to 153, wherein G at position 1 and/or Sat position 2 of SEQ ID NOs: 144 to 153 are optionally missing and wherein L at the second last position and/or N at the last position of SEQ ID NOs: 144, 145 and 148 to 150 are optionally exchanged by A. In one embodiment, said ankyrin repeat domain comprises an amino acid sequence with at least 90% amino acid sequence identity with any one of SEQ ID NOs: 144 to 153, wherein G at position 1 and/or S at position 2 of SEQ ID NOs: 144 to 153 are optionally missing and wherein L at the second last position and/or N at the last position of SEQ ID NOs: 144, 145 and 148 to 150 are optionally exchanged by A. Thus, in one embodiment, said ankyrin repeat domain comprises an amino acid sequence with at least 93% amino acid sequence identity with any one of SEQ ID NOs: 144 to 153; and in a further embodiment, said ankyrin repeat domain comprises an amino acid sequence with at least 95% amino acid sequence identity with any one of SEQ ID NOs: 144 to 153. In one embodiment, said ankyrin repeat domain comprises an amino acid sequence with at least 98% amino acid sequence identity with any one of SEQ ID NOs: 144 to 153; and in one embodiment, said ankyrin repeat domain comprises an amino acid sequence of any one of SEQ ID NOs: 144 to 153. Thus, in one embodiment, said recombinant binding protein comprises an ankyrin repeat domain having binding specificity for FAP, wherein said ankyrin repeat domain comprises an amino acid sequence selected from SEQ ID NOs: 144 to 153, wherein G at position 1 and/or Sat position 2 of SEQ ID NOs: 144 to 153 are optionally missing and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 10⁷M, or below 10⁻⁸M, or below 10⁻⁹M, or below 5×10⁻¹⁰M, or below 3×10⁻¹⁰M, or below 2×10⁻¹⁰M. Thus, in one embodiment, said ankyrin repeat domain binds human FAP in PBS with a dissociation constant (K_{D}) below 10⁻⁷M. In another embodiment, said ankyrin repeat domain binds human FAP in PBS with a dissociation constant (K_{D}) below 10⁻⁸M; and in a further embodiment, said ankyrin repeat domain binds human FAP in PBS with a dissociation constant (K_{D}) below 10⁻⁹M. In one embodiment, said ankyrin repeat domain binds human FAP in PBS with a dissociation constant (K_{D}) below 5×10⁻¹⁰M. In one embodiment, said ankyrin repeat domain binds human FAP in PBS with a dissociation constant (K_{D}) below 3×10⁻¹⁰M, and in one embodiment, said ankyrin repeat domain binds human FAP in PBS with a dissociation constant (K_{D}) below 2×10⁻¹⁰M.

In one embodiment, said recombinant binding protein comprises an ankyrin repeat domain having binding specificity for FAP, wherein said ankyrin repeat domain binds human FAP in PBS with a dissociation constant (K_{D}) below 10⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 1 to 35 and 144 to 153. In another embodiment, said ankyrin repeat domain comprises an amino acid sequence with at least 93% amino acid sequence identity with any one of SEQ ID NOs: 1 to 35 and 144 to 153; and in a further embodiment, said ankyrin repeat domain comprises an amino acid sequence with at least 95% amino acid sequence identity with any one of SEQ ID NOs: 1 to 35 and 144 to 153. In one embodiment, said ankyrin repeat domain comprises an amino acid sequence with at least 98% amino acid sequence identity with any one of SEQ ID NOs: 1 to 35 and 144 to 153; and in one embodiment, said ankyrin repeat domain comprises an amino acid sequence of any one of SEQ ID NOs: 1 to 35 and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 9, 11 to 35, and 144 to 153, wherein G at position 1 and/or Sat position 2 of SEQ ID NOs: 1 to 9, 11 to 35, and 144 to 153 are optionally missing, and wherein L at the second last position and/or N at the last position of SEQ ID NOs: 1 to 9, 11 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 1 to 9, 11 to 35, and 144 to 153. In another embodiment, said ankyrin repeat domain comprises an amino acid sequence with at least 93% amino acid sequence identity with any one of SEQ ID NOs: 1 to 9, 11 to 35, and 144 to 153; and in a further embodiment, said ankyrin repeat domain comprises an amino acid sequence with at least 95% amino acid sequence identity with any one of SEQ ID NOs: 1 to 9, 11 to 35, and 144 to 153. In one embodiment, said ankyrin repeat domain comprises an amino acid sequence with at least 98% amino acid sequence identity with any one of SEQ ID NOs: 1 to 9, 11 to 35, and 144 to 153; and in one embodiment, said ankyrin repeat domain comprises an amino acid sequence of any one of SEQ ID NOs: 1 to 9, 11 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 5 to 9, 11 to 21, 25 to 29, 31 to 35, and 144 to 153, wherein G at position 1 and/or S at position 2 of SEQ ID NOs: 5 to 9, 11 to 21, 25 to 29, 31 to 35, and 144 to 153 are optionally missing, and wherein L at the second last position and/or N at the last position of SEQ ID NOs: 5 to 9, 11 to 21, 25 to 29, 31 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 5 to 9, 11 to 21, 25 to 29, 31 to 35, and 144 to 153. In another embodiment, said ankyrin repeat domain comprises an amino acid sequence with at least 93% amino acid sequence identity with any one of SEQ ID NOs: 5 to 9, 11 to 21, 25 to 29, 31 to 35, and 144 to 153; and in a further embodiment, said ankyrin repeat domain comprises an amino acid sequence with at least 95% amino acid sequence identity with any one of SEQ ID NOs: 5 to 9, 11 to 21, 25 to 29, 31 to 35, and 144 to 153. In one embodiment, said ankyrin repeat domain comprises an amino acid sequence with at least 98% amino acid sequence identity with any one of SEQ ID NOs: 5 to 9, 11 to 21, 25 to 29, 31 to 35, and 144 to 153; and in one embodiment, said ankyrin repeat domain comprises an amino acid sequence of any one of SEQ ID NOs: 5 to 9, 11 to 21, 25 to 29, 31 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 5×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 6, 8, 9, 11 to 20, 25 to 29, 31, 33 to 35, and 144 to 153, wherein G at position 1 and/or Sat position 2 of SEQ ID NOs: 6, 8, 9, 11 to 20, 25 to 29, 31, 33 to 35, and 144 to 153 are optionally missing, and wherein L at the second last position and/or N at the last position of SEQ ID NOs: 6, 8, 9, 11 to 20, 25 to 29, 31, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 6, 8, 9, 11 to 20, 25 to 29, 31, 33 to 35, and 144 to 153. In another embodiment, said ankyrin repeat domain comprises an amino acid sequence with at least 93% amino acid sequence identity with any one of SEQ ID NOs: 6, 8, 9, 11 to 20, 25 to 29, 31, 33 to 35, and 144 to 153; and in a further embodiment, said ankyrin repeat domain comprises an amino acid sequence with at least 95% amino acid sequence identity with any one of SEQ ID NOs: 6, 8, 9, 11 to 20, 25 to 29, 31, 33 to 35, and 144 to 153. In one embodiment, said ankyrin repeat domain comprises an amino acid sequence with at least 98% amino acid sequence identity with any one of SEQ ID NOs: 6, 8, 9, 11 to 20, 25 to 29, 31, 33 to 35, and 144 to 153; and in one embodiment, said ankyrin repeat domain comprises an amino acid sequence of any one of SEQ ID NOs: 6, 8, 9, 11 to 20, 25 to 29, 31, 33 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 8, 9, 11 to 20, 26, 28, 29, 31, 33 to 35, and 144 to 153, wherein G at position 1 and/or S at position 2 of SEQ ID NOs: 8, 9, 11 to 20, 26, 28, 29, 31, 33 to 35, and 144 to 153, are optionally missing, and wherein L at the second last position and/or N at the last position of SEQ ID NOs: 8, 9, 11 to 20, 26, 28, 29, 31, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 8, 9, 11 to 20, 26, 28, 29, 31, 33 to 35, and 144 to 153. In another embodiment, said ankyrin repeat domain comprises an amino acid sequence with at least 93% amino acid sequence identity with any one of SEQ ID NOs: 8, 9, 11 to 20, 26, 28, 29, 31, 33 to 35, and 144 to 153; and in a further embodiment, said ankyrin repeat domain comprises an amino acid sequence with at least 95% amino acid sequence identity with any one of SEQ ID NOs: 8, 9, 11 to 20, 26, 28, 29, 31, 33 to 35, and 144 to 153. In one embodiment, said ankyrin repeat domain comprises an amino acid sequence with at least 98% amino acid sequence identity with any one of SEQ ID NOs: 8, 9, 11 to 20, 26, 28, 29, 31, 33 to 35, and 144 to 153; and in one embodiment, said ankyrin repeat domain comprises an amino acid sequence of any one of SEQ ID NOs: 8, 9, 11 to 20, 26, 28, 29, 31, 33 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 8, 9, 11 to 20, 26, 28, 29, 31 and 33 to 35, wherein G at position 1 and/or S at position 2 of SEQ ID NOs: 8, 9, 11 to 20, 26, 28, 29, 31 and 33 to 35, are optionally missing, and wherein L at the second last position and/or N at the last position of SEQ ID NOs: 8, 9, 11 to 20, 26, 28, 29, 31 and 33 are optionally exchanged by A. Thus, in one embodiment, said ankyrin repeat domain comprises an amino acid sequence with at least 90% amino acid sequence identity with any one of SEQ ID NOs: 8, 9, 11 to 20, 26, 28, 29, 31 and 33 to 35. In another embodiment, said ankyrin repeat domain comprises an amino acid sequence with at least 93% amino acid sequence identity with any one of SEQ ID NOs: 8, 9, 11 to 20, 26, 28, 29, 31 and 33 to 35; and in a further embodiment, said ankyrin repeat domain comprises an amino acid sequence with at least 95% amino acid sequence identity with any one of SEQ ID NOs: 8, 9, 11 to 20, 26, 28, 29, 31 and 33 to 35. In one embodiment, said ankyrin repeat domain comprises an amino acid sequence with at least 98% amino acid sequence identity with any one of SEQ ID NOs: 8, 9, 11 to 20, 26, 28, 29, 31 and 33 to 35; and in one embodiment, said ankyrin repeat domain comprises an amino acid sequence of any one of SEQ ID NOs: 8, 9, 11 to 20, 26, 28, 29, 31 and 33 to 35.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or S at position 2 of SEQ ID NOs: 18, 19, 26, 33 to 35, and 144 to 153, are optionally missing, and wherein L at the second last position and/or N at the last position of SEQ ID NOs: 18, 19, 26, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 18, 19, 26, 33 to 35, and 144 to 153. In another embodiment, said ankyrin repeat domain comprises an amino acid sequence with at least 93% amino acid sequence identity with any one of SEQ ID NOs: 18, 19, 26, 33 to 35, and 144 to 153; and in a further embodiment, said ankyrin repeat domain comprises an amino acid sequence with at least 95% amino acid sequence identity with any one of SEQ ID NOs: 18, 19, 26, 33 to 35, and 144 to 153. In one embodiment, said ankyrin repeat domain comprises an amino acid sequence with at least 98% amino acid sequence identity with any one of SEQ ID NOs: 18, 19, 26, 33 to 35, and 144 to 153; and in one embodiment, said ankyrin repeat domain comprises an amino acid sequence of any one of SEQ ID NOs: 18, 19, 26, 33 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26 and 33 to 35, wherein G at position 1 and/or S at position 2 of SEQ ID NOs: 18, 19, 26 and 33 to 35 are optionally missing, and wherein L at the second last position and/or N at the last position of SEQ ID NOs: 18, 19, 26 and 33 are optionally exchanged by A. Thus, in one embodiment, said ankyrin repeat domain comprises an amino acid sequence with at least 90% amino acid sequence identity with any one of SEQ ID NOs: 18, 19, 26 and 33 to 35. In another embodiment, said ankyrin repeat domain comprises an amino acid sequence with at least 93% amino acid sequence identity with any one of SEQ ID NOs: 18, 19, 26 and 33 to 35; and in a further embodiment, said ankyrin repeat domain comprises an amino acid sequence with at least 95% amino acid sequence identity with any one of SEQ ID NOs: 18, 19, 26 and 33 to 35. In one embodiment, said ankyrin repeat domain comprises an amino acid sequence with at least 98% amino acid sequence identity with any one of SEQ ID NOs: 18, 19, 26 and 33 to 35; and in one embodiment, said ankyrin repeat domain comprises an amino acid sequence of any one of SEQ ID NOs: 18, 19, 26 and 33 to 35.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or Sat position 2 of SEQ ID NO: 18 are optionally missing, and wherein L at the second last position and/or N at the last position of SEQ ID NO: 18 are optionally exchanged by A. Thus, in one embodiment, said ankyrin repeat domain comprises an amino acid sequence with at least 90% amino acid sequence identity with SEQ ID NO: 18. In another embodiment, said ankyrin repeat domain comprises an amino acid sequence with at least 93% amino acid sequence identity with SEQ ID NO: 18; and in a further embodiment, said ankyrin repeat domain comprises an amino acid sequence with at least 95% amino acid sequence identity with SEQ ID NO: 18. In one embodiment, said ankyrin repeat domain comprises an amino acid sequence with at least 98% amino acid sequence identity with SEQ ID NO: 18; and in one embodiment, said ankyrin repeat domain comprises the amino acid sequence of SEQ ID NO: 18.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 19, wherein G at position 1 and/or Sat position 2 of SEQ ID NO: 19 are optionally missing, and wherein L at the second last position and/or N at the last position of SEQ ID NO: 19 are optionally exchanged by A. Thus, in one embodiment, said ankyrin repeat domain comprises an amino acid sequence with at least 90% amino acid sequence identity with SEQ ID NO: 19. In another embodiment, said ankyrin repeat domain comprises an amino acid sequence with at least 93% amino acid sequence identity with SEQ ID NO: 19; and in a further embodiment, said ankyrin repeat domain comprises an amino acid sequence with at least 95% amino acid sequence identity with SEQ ID NO: 19. In one embodiment, said ankyrin repeat domain comprises an amino acid sequence with at least 98% amino acid sequence identity with SEQ ID NO: 19; and in one embodiment, said ankyrin repeat domain comprises the amino acid sequence of SEQ ID NO: 19.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing. Thus, in one embodiment, said ankyrin repeat domain comprises an amino acid sequence with at least 90% amino acid sequence identity with SEQ ID NO: 34. In another embodiment, said ankyrin repeat domain comprises an amino acid sequence with at least 93% amino acid sequence identity with SEQ ID NO: 34; and in a further embodiment, said ankyrin repeat domain comprises an amino acid sequence with at least 95% amino acid sequence identity with SEQ ID NO: 34. In one embodiment, said ankyrin repeat domain comprises an amino acid sequence with at least 98% amino acid sequence identity with SEQ ID NO: 34; and in one embodiment, said ankyrin repeat domain comprises the amino acid sequence of SEQ ID NO: 34.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence with at least 90% amino acid sequence identity with any one of SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A. Thus, in one embodiment, said ankyrin repeat domain comprises an amino acid sequence with at least 93% amino acid sequence identity with any one of SEQ ID NOs: 144 to 153; and in a further embodiment, said ankyrin repeat domain comprises an amino acid sequence with at least 95% amino acid sequence identity with any one of SEQ ID NOs: 144 to 153. In one embodiment, said ankyrin repeat domain comprises an amino acid sequence with at least 98% amino acid sequence identity with any one of SEQ ID NOs: 144 to 153; and in one embodiment, said ankyrin repeat domain comprises an amino acid sequence of any one of SEQ ID NOs: 144 to 153. Thus, in one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain comprises an amino acid sequence selected from SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 2×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 8, 9, 11 to 20, 26, 28, 31 and 34, wherein G at position 1 and/or S at position 2 of SEQ ID NOs: 8, 9, 11 to 20, 26, 28, 31 and 34, are optionally missing, and wherein L at the second last position and/or N at the last position of SEQ ID NOs: 8, 9, 11 to 20, 26, 28 and 31 are optionally exchanged by A. Thus, in one embodiment, said ankyrin repeat domain comprises an amino acid sequence with at least 90% amino acid sequence identity with any one of SEQ ID NOs: 8, 9, 11 to 20, 26, 28, 31 and 34. In another embodiment, said ankyrin repeat domain comprises an amino acid sequence with at least 93% amino acid sequence identity with any one of SEQ ID NOs: 8, 9, 11 to 20, 26, 28, 31 and 34; and in a further embodiment, said ankyrin repeat domain comprises an amino acid sequence with at least 95% amino acid sequence identity with any one of SEQ ID NOs: 8, 9, 11 to 20, 26, 28, 31 and 34. In one embodiment, said ankyrin repeat domain comprises an amino acid sequence with at least 98% amino acid sequence identity with any one of SEQ ID NOs: 8, 9, 11 to 20, 26, 28, 31 and 34; and in one embodiment, said ankyrin repeat domain comprises an amino acid sequence of any one of SEQ ID NOs: 8, 9, 11 to 20, 26, 28, 31 and 34.

In one embodiment, said recombinant binding protein comprises an ankyrin repeat domain having binding specificity for FAP, wherein said ankyrin repeat domain binds human FAP in PBS with a dissociation constant (K_{D}) below 2×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26 and 33 to 35, wherein G at position 1 and/or S at position 2 of SEQ ID NOs: 18, 19, 26 and 33 to 35 are optionally missing, and wherein L at the second last position and/or N at the last position of SEQ ID NOs: 18, 19, 26 and 33 are optionally exchanged by A. Thus, in one embodiment, said ankyrin repeat domain comprises an amino acid sequence with at least 90% amino acid sequence identity with any one of SEQ ID NOs: 18, 19, 26 and 33 to 35. In another embodiment, said ankyrin repeat domain comprises an amino acid sequence with at least 93% amino acid sequence identity with any one of SEQ ID NOs: 18, 19, 26 and 33 to 35; and in a further embodiment, said ankyrin repeat domain comprises an amino acid sequence with at least 95% amino acid sequence identity with any one of SEQ ID NOs: 18, 19, 26 and 33 to 35. In one embodiment, said ankyrin repeat domain comprises an amino acid sequence with at least 98% amino acid sequence identity with any one of SEQ ID NOs: 18, 19, 26 and 33 to 35; and in one embodiment, said ankyrin repeat domain comprises an amino acid sequence of any one of SEQ ID NOs: 18, 19, 26 and 33 to 35.

A typical and preferred determination of dissociation constants (K_{D}) of the inventive recombinant binding proteins with binding specificity for FAP by Surface Plasmon Resonance (SPR) analysis is described in Example 2. Thus, in one embodiment said binding specificity for FAP of the inventive recombinant binding proteins are determined in PBS by Surface Plasmon Resonance (SPR). In one embodiment said binding specificity for FAP of the inventive recombinant binding proteins are determined in PBS by Surface Plasmon Resonance (SPR) as described in Example 2.

In one embodiment, said recombinant binding protein comprises an ankyrin repeat domain having binding specificity for FAP, wherein said ankyrin repeat domain binds human FAP-expressing WI38 cells with an EC₅₀ below 10⁻⁷M, or below 10⁻⁸M, or about or below 10⁻⁹M, or about or below 5×10⁻¹⁰M, or about or below 3×10⁻¹° M, or about or below 2×10⁻¹⁰M. Thus, in one embodiment, said ankyrin repeat domain binds human FAP-expressing WI38 cells with an EC₅₀ below 10⁻⁷M. In another embodiment, said ankyrin repeat domain binds human FAP-expressing WI38 cells with an EC₅₀ below 10⁻⁸M, and in a further embodiment, said ankyrin repeat domain binds human FAP-expressing WI38 cells with an EC₅₀ about or below 10⁻⁹M. In one embodiment, said ankyrin repeat domain binds human FAP-expressing WI38 cells with an EC₅₀ about or below 5×10⁻¹⁰M. In one embodiment, said ankyrin repeat domain binds human FAP-expressing WI38 cells with an EC₅₀ about or below 3×10⁻¹⁰M, and in another embodiment said ankyrin repeat domain binds human FAP-expressing WI38 cells with an EC₅₀ about or below 2×10⁻¹⁰M.

In one embodiment, said recombinant binding protein comprises an ankyrin repeat domain having binding specificity for FAP, wherein said ankyrin repeat domain binds human FAP-expressing WI38 cells with an EC₅₀ below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 1 to 35 and 144 to 153. In another embodiment, said ankyrin repeat domain comprises an amino acid sequence with at least 93% amino acid sequence identity with any one of SEQ ID NOs: 1 to 35 and 144 to 153; and in a further embodiment, said ankyrin repeat domain comprises an amino acid sequence with at least 95% amino acid sequence identity with any one of SEQ ID NOs: 1 to 35 and 144 to 153. In one embodiment, said ankyrin repeat domain comprises an amino acid sequence with at least 98% amino acid sequence identity with any one of SEQ ID NOs: 1 to 35 and 144 to 153; and in one embodiment, said ankyrin repeat domain comprises an amino acid sequence of any one of SEQ ID NOs: 1 to 35 and 144 to 153.

In one embodiment, said recombinant binding protein comprises an ankyrin repeat domain having binding specificity for FAP, wherein said ankyrin repeat domain binds human FAP-expressing WI38 cells with an EC₅₀ below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 1 to 35 and 144 to 153. In another embodiment, said ankyrin repeat domain comprises an amino acid sequence with at least 93% amino acid sequence identity with any one of SEQ ID NOs: 1 to 35 and 144 to 153; and in a further embodiment, said ankyrin repeat domain comprises an amino acid sequence with at least 95% amino acid sequence identity with any one of SEQ ID NOs: 1 to 35 and 144 to 153. In one embodiment, said ankyrin repeat domain comprises an amino acid sequence with at least 98% amino acid sequence identity with any one of SEQ ID NOs: 1 to 35 and 144 to 153; and in one embodiment, said ankyrin repeat domain comprises an amino acid sequence of any one of SEQ ID NOs: 1 to 35 and 144 to 153.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 4, 5, 9 to 15, 18, 19, 26 to 30, 32 to 35, and 144 to 153, wherein G at position 1 and/or Sat position 2 of SEQ ID NOs: 4, 5, 9 to 15, 18, 19, 26 to 30, 32 to 35 and 144 to 153 are optionally missing, and wherein L at the second last position and/or N at the last position of SEQ ID NOs: 4, 5, 9 to 15, 18, 19, 26 to 30, 32 to 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 4, 5, 9 to 15, 18, 19, 26 to 30, 32 to 35, and 144 to 153. In another embodiment, said ankyrin repeat domain comprises an amino acid sequence with at least 93% amino acid sequence identity with any one of SEQ ID NOs: 4, 5, 9 to 15, 18, 19, 26 to 30, 32 to 35, and 144 to 153; and in a further embodiment, said ankyrin repeat domain comprises an amino acid sequence with at least 95% amino acid sequence identity with any one of SEQ ID NOs: 4, 5, 9 to 15, 18, 19, 26 to 30, 32 to 35, and 144 to 153. In one embodiment, said ankyrin repeat domain comprises an amino acid sequence with at least 98% amino acid sequence identity with any one of SEQ ID NOs: 4, 5, 9 to 15, 18, 19, 26 to 30, 32 to 35, and 144 to 153; and in one embodiment, said ankyrin repeat domain comprises an amino acid sequence of any one of SEQ ID NOs: 4, 5, 9 to 15, 18, 19, 26 to 30, 32 to 35, and 144 to 153.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 4, 5, 9 to 15, 18, 19, 26 to 30, and 32 to 35, wherein G at position 1 and/or Sat position 2 of SEQ ID NOs: 4, 5, 9 to 15, 18, 19, 26 to 30, and 32 to 35 are optionally missing, and wherein L at the second last position and/or N at the last position of SEQ ID NOs: 4, 5, 9 to 15, 18, 19, 26 to 30, and 32 to 33 are optionally exchanged by A. Thus, in one embodiment, said ankyrin repeat domain comprises an amino acid sequence with at least 90% amino acid sequence identity with any one of SEQ ID NOs: 4, 5, 9 to 15, 18, 19, 26 to 30, and 32 to 35. In another embodiment, said ankyrin repeat domain comprises an amino acid sequence with at least 93% amino acid sequence identity with any one of SEQ ID NOs: 4, 5, 9 to 15, 18, 19, 26 to 30, and 32 to 35; and in a further embodiment, said ankyrin repeat domain comprises an amino acid sequence with at least 95% amino acid sequence identity with any one of SEQ ID NOs: 4, 5, 9 to 15, 18, 19, 26 to 30, and 32 to 35. In one embodiment, said ankyrin repeat domain comprises an amino acid sequence with at least 98% amino acid sequence identity with any one of SEQ ID NOs: 4, 5, 9 to 15, 18, 19, 26 to 30, and 32 to 35; and in one embodiment, said ankyrin repeat domain comprises an amino acid sequence of any one of SEQ ID NOs: 4, 5, 9 to 15, 18, 19, 26 to 30, and 32 to 35.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or S at position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 18, 19, 26, 33 to 35, and 144 to 153. In another embodiment, said ankyrin repeat domain comprises an amino acid sequence with at least 93% amino acid sequence identity with any one of SEQ ID NOs: 18, 19, 26, 33 to 35, and 144 to 153; and in a further embodiment, said ankyrin repeat domain comprises an amino acid sequence with at least 95% amino acid sequence identity with any one of SEQ ID NOs: 18, 19, 26, 33 to 35, and 144 to 153. In one embodiment, said ankyrin repeat domain comprises an amino acid sequence with at least 98% amino acid sequence identity with any one of SEQ ID NOs: 18, 19, 26, 33 to 35, and 144 to 153; and in one embodiment, said ankyrin repeat domain comprises an amino acid sequence of any one of SEQ ID NOs: 18, 19, 26, 33 to 35, and 144 to 153.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26 and 33 to 35, wherein G at position 1 and/or S at position 2 of SEQ ID NOs: 18, 19, 26 and 33 to 35 are optionally missing, and wherein L at the second last position and/or N at the last position of SEQ ID NOs: 18, 19, 26 and 33 are optionally exchanged by A. Thus, in one embodiment, said ankyrin repeat domain comprises an amino acid sequence with at least 90% amino acid sequence identity with any one of SEQ ID NOs: 18, 19, 26 and 33 to 35. In another embodiment, said ankyrin repeat domain comprises an amino acid sequence with at least 93% amino acid sequence identity with any one of SEQ ID NOs: 18, 19, 26 and 33 to 35; and in a further embodiment, said ankyrin repeat domain comprises an amino acid sequence with at least 95% amino acid sequence identity with any one of SEQ ID NOs: 18, 19, 26 and 33 to 35. In one embodiment, said ankyrin repeat domain comprises an amino acid sequence with at least 98% amino acid sequence identity with any one of SEQ ID NOs: 18, 19, 26 and 33 to 35; and in one embodiment, said ankyrin repeat domain comprises an amino acid sequence of any one of SEQ ID NOs: 18, 19, 26 and 33 to 35.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing. Thus, in one embodiment, said ankyrin repeat domain comprises an amino acid sequence with at least 90% amino acid sequence identity with SEQ ID NO: 34. In another embodiment, said ankyrin repeat domain comprises an amino acid sequence with at least 93% amino acid sequence identity with SEQ ID NO: 34; and in a further embodiment, said ankyrin repeat domain comprises an amino acid sequence with at least 95% amino acid sequence identity with SEQ ID NO: 34. In one embodiment, said ankyrin repeat domain comprises an amino acid sequence with at least 98% amino acid sequence identity with SEQ ID NO: 34; and in one embodiment, said ankyrin repeat domain comprises the amino acid sequence of SEQ ID NO: 34.

In one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises an amino acid sequence with at least 90% with any one of SEQ ID NOs: 144 to 153, wherein G at position 1 and/or Sat position 2 of SEQ ID NOs: 144 to 153 are optionally missing, and wherein L at the second last position and/or N at the last position of SEQ ID NOs: 144, 145 and 148 to 150 are optionally exchanged by A. Thus, in one embodiment, said ankyrin repeat domain comprises an amino acid sequence with at least 93% amino acid sequence identity with any one of SEQ ID NOs: 144 to 153; and in a further embodiment, said ankyrin repeat domain comprises an amino acid sequence with at least 95% amino acid sequence identity with any one of SEQ ID NOs: 144 to 153. In one embodiment, said ankyrin repeat domain comprises an amino acid sequence with at least 98% amino acid sequence identity with any one of SEQ ID NOs: 144 to 153; and in one embodiment, said ankyrin repeat domain comprises an amino acid sequence of any one of SEQ ID NOs: 144 to 153. Thus, in one embodiment, said recombinant binding protein comprises an ankyrin repeat domain having binding specificity for FAP, wherein said ankyrin repeat domain binds human FAP-expressing WI38 cells with an EC₅₀ about or below 10⁻⁹M, and wherein said ankyrin repeat domain comprises the amino acid sequence of SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein said ankyrin repeat domain binds human FAP-expressing WI38 cells with an EC₅₀ about or below 5×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9 to 11, 13 to 15, 18, 19, 26 and 33, wherein G at position 1 and/or Sat position 2 of SEQ ID NOs: 9 to 11, 13 to 15, 18, 19, 26 and 33 are optionally missing, and wherein L at the second last position and/or N at the last position of SEQ ID NOs: 9 to 11, 13 to 15, 18, 19, 26 and 33 are optionally exchanged by A. Thus, in one embodiment, said ankyrin repeat domain comprises an amino acid sequence with at least 90% amino acid sequence identity with any one of SEQ ID NOs: 9 to 11, 13 to 15, 18, 19, 26 and 33. In another embodiment, said ankyrin repeat domain comprises an amino acid sequence with at least 93% amino acid sequence identity with any one of SEQ ID NOs: 9 to 11, 13 to 15, 18, 19, 26 and 33; and in a further embodiment, said ankyrin repeat domain comprises an amino acid sequence with at least 95% amino acid sequence identity with any one of SEQ ID NOs: 9 to 11, 13 to 15, 18, 19, 26 and 33. In one embodiment, said ankyrin repeat domain comprises an amino acid sequence with at least 98% amino acid sequence identity with any one of SEQ ID NOs: 9 to 11, 13 to 15, 18, 19, 26 and 33; and in one embodiment, said ankyrin repeat domain comprises an amino acid sequence of any one of SEQ ID NOs: 9 to 11, 13 to 15, 18, 19, 26 and 33.

In one embodiment, said recombinant binding protein comprises an ankyrin repeat domain having binding specificity for FAP, wherein said ankyrin repeat domain binds human FAP-expressing WI38 cells with an EC₅₀ about or below 5×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A. Thus, in one embodiment, said ankyrin repeat domain comprises an amino acid sequence with at least 90% amino acid sequence identity with SEQ ID NO: 18. In another embodiment, said ankyrin repeat domain comprises an amino acid sequence with at least 93% amino acid sequence identity with SEQ ID NO: 18; and in a further embodiment, said ankyrin repeat domain comprises an amino acid sequence with at least 95% amino acid sequence identity with SEQ ID NO: 18. In one embodiment, said ankyrin repeat domain comprises an amino acid sequence with at least 98% amino acid sequence identity with SEQ ID NO: 18; and in one embodiment, said ankyrin repeat domain comprises the amino acid sequence of SEQ ID NO: 18.

In one embodiment, said recombinant binding protein comprises an ankyrin repeat domain having binding specificity for FAP, wherein said ankyrin repeat domain binds human FAP-expressing WI38 cells with an EC₅₀ about or below 5×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 19, wherein G at position 1 and/or S at position 2 of SEQ ID NO: 19 are optionally missing, and wherein L at the second last position and/or N at the last position of SEQ ID NO: 19 are optionally exchanged by A. Thus, in one embodiment, said ankyrin repeat domain comprises an amino acid sequence with at least 90% amino acid sequence identity with SEQ ID NO: 19. In another embodiment, said ankyrin repeat domain comprises an amino acid sequence with at least 93% amino acid sequence identity with SEQ ID NO: 19; and in a further embodiment, said ankyrin repeat domain comprises an amino acid sequence with at least 95% amino acid sequence identity with SEQ ID NO: 19. In one embodiment, said ankyrin repeat domain comprises an amino acid sequence with at least 98% amino acid sequence identity with SEQ ID NO: 19; and in one embodiment, said ankyrin repeat domain comprises the amino acid sequence of SEQ ID NO: 19.

In one embodiment, said recombinant binding protein comprises an ankyrin repeat domain having binding specificity for FAP, wherein said ankyrin repeat domain binds human FAP-expressing WI38 cells with an EC₅₀ about or below 5×10⁻¹⁰M, and wherein said ankyrin repeat domain comprises an amino acid sequence with at least 90% with any one of SEQ ID NOs: 144 to 153, wherein G at position 1 and/or Sat position 2 of SEQ ID NOs: 144 to 153 are optionally missing, and wherein L at the second last position and/or N at the last position of SEQ ID NOs: 144, 145 and 148 to 150 are optionally exchanged by A. Thus, in one embodiment, said ankyrin repeat domain comprises an amino acid sequence with at least 93% amino acid sequence identity with any one of SEQ ID NOs: 144 to 153; and in a further embodiment, said ankyrin repeat domain comprises an amino acid sequence with at least 95% amino acid sequence identity with any one of SEQ ID NOs: 144 to 153. In one embodiment, said ankyrin repeat domain comprises an amino acid sequence with at least 98% amino acid sequence identity with any one of SEQ ID NOs: 144 to 153; and in one embodiment, said ankyrin repeat domain comprises an amino acid sequence of any one of SEQ ID NOs: 144 to 153. Thus, in one embodiment, said recombinant binding protein comprises an ankyrin repeat domain having binding specificity for FAP, wherein said ankyrin repeat domain binds human FAP-expressing WI38 cells with an EC₅₀ about or below 5×10⁻¹⁰M, and wherein said ankyrin repeat domain comprises an amino acid sequence selected from SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein said ankyrin repeat domain binds human FAP-expressing WI38 cells with an EC₅₀ about or below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9, 13 to 15, 18, 19, 26 and 33, wherein G at position 1 and/or Sat position 2 of SEQ ID NOs: 9, 13 to 15, 18, 19, 26 and 33 are optionally missing, and wherein L at the second last position and/or N at the last position of SEQ ID NOs: 9, 13 to 15, 18, 19, 26 and 33 are optionally exchanged by A. Thus, in one embodiment, said ankyrin repeat domain comprises an amino acid sequence with at least 90% amino acid sequence identity with any one of SEQ ID NOs: 9, 13 to 15, 18, 19, 26 and 33. In another embodiment, said ankyrin repeat domain comprises an amino acid sequence with at least 93% amino acid sequence identity with any one of SEQ ID NOs: 9, 13 to 15, 18, 19, 26 and 33; and in a further embodiment, said ankyrin repeat domain comprises an amino acid sequence with at least 95% amino acid sequence identity with any one of SEQ ID NOs: 9, 13 to 15, 18, 19, 26 and 33. In one embodiment, said ankyrin repeat domain comprises an amino acid sequence with at least 98% amino acid sequence identity with any one of SEQ ID NOs: 9, 13 to 15, 18, 19, 26 and 33; and in one embodiment, said ankyrin repeat domain comprises an amino acid sequence of any one of SEQ ID NOs: 9, 13 to 15, 18, 19, 26 and 33.

In one embodiment, said recombinant binding protein comprises an ankyrin repeat domain having binding specificity for FAP, wherein said ankyrin repeat domain binds human FAP-expressing WI38 cells with an EC₅₀ about or below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A. Thus, in one embodiment, said ankyrin repeat domain comprises an amino acid sequence with at least 90% amino acid sequence identity with SEQ ID NO: 18. In another embodiment, said ankyrin repeat domain comprises an amino acid sequence with at least 93% amino acid sequence identity with SEQ ID NO: 18; and in a further embodiment, said ankyrin repeat domain comprises an amino acid sequence with at least 95% amino acid sequence identity with SEQ ID NO: 18. In one embodiment, said ankyrin repeat domain comprises an amino acid sequence with at least 98% amino acid sequence identity with SEQ ID NO: 18; and in one embodiment, said ankyrin repeat domain comprises the amino acid sequence of SEQ ID NO: 18.

In one embodiment, said recombinant binding protein comprises an ankyrin repeat domain having binding specificity for FAP, wherein said ankyrin repeat domain binds human FAP-expressing WI38 cells with an EC₅₀ about or below 2×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 26 and 33, wherein G at position 1 and/or S at position 2 of SEQ ID NOs: 18, 26 and 33 are optionally missing, and wherein L at the second last position and/or N at the last position of SEQ ID NOs: 18, 26 and 33 are optionally exchanged by A. Thus, in one embodiment, said ankyrin repeat domain comprises an amino acid sequence with at least 90% amino acid sequence identity with any one of SEQ ID NOs: 18, 26 and 33. In another embodiment, said ankyrin repeat domain comprises an amino acid sequence with at least 93% amino acid sequence identity with any one of SEQ ID NOs: 18, 26 and 33; and in a further embodiment, said ankyrin repeat domain comprises an amino acid sequence with at least 95% amino acid sequence identity with any one of SEQ ID NOs: 18, 26 and 33. In one embodiment, said ankyrin repeat domain comprises an amino acid sequence with at least 98% amino acid sequence identity with any one of SEQ ID NOs: 18, 26 and 33; and in one embodiment, said ankyrin repeat domain comprises an amino acid sequence of any one of SEQ ID NOs: 18, 26 and 33.

A typical and preferred determination of binding of the ankyrin repeat domains and recombinant binding proteins of the present invention, respectively, to human FAP-expressing WI38 cells and the determination of the EC₅₀ is described in Example 3. Thus, in one embodiment said binding to human FAP-expressing WI38 cells of the inventive ankyrin repeat domains and recombinant binding proteins are determined as described in Example 3. In one embodiment said binding to human FAP-expressing WI38 cells of the inventive ankyrin repeat domains and recombinant binding proteins are determined by FACS analysis as described in Example 3.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁷M, and wherein said ankyrin repeat domain binds human FAP-expressing WI38 cells with an EC₅₀ below 10⁻⁷M.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁷M, wherein said ankyrin repeat domain binds human FAP-expressing WI38 cells with an EC₅₀ below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 10⁷M, and wherein said ankyrin repeat domain binds human FAP-expressing WI38 cells with an EC₅₀ below 10⁻⁸M.

In one embodiment, said recombinant binding protein comprises an ankyrin repeat domain having binding specificity for FAP, wherein said ankyrin repeat domain binds human FAP in PBS with a dissociation constant (K_{D}) below 10⁷M, wherein said ankyrin repeat domain binds human FAP-expressing WI38 cells with an EC₅₀ below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 35 and 144 to 153, wherein G at position 1 and/or S at position 2 of SEQ ID NOs: 1 to 35 and 144 to 153 are optionally missing, and wherein L at the second last position and/or N at the last position of SEQ ID NOs: 1 to 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁸M, and wherein said ankyrin repeat domain binds human FAP-expressing WI38 cells with an EC₅₀ below 10⁻⁸M.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁸M, wherein said ankyrin repeat domain binds human FAP-expressing WI38 cells with an EC₅₀ below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 9, 11 to 35, and 144 to 153, wherein G at position 1 and/or Sat position 2 of SEQ ID NOs: 1 to 9, 11 to 35, and 144 to 153 are optionally missing, and wherein L at the second last position and/or N at the last position of SEQ ID NOs: 1 to 9, 11 to 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁹M, wherein said ankyrin repeat domain binds human FAP-expressing WI38 cells with an EC₅₀ below 10⁻⁸M.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁹M, wherein said ankyrin repeat domain binds human FAP-expressing WI38 cells with an EC₅₀ below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 5 to 9, 11 to 21, 25 to 29, 31 to 35, and 144 to 153, wherein G at position 1 and/or S at position 2 of SEQ ID NOs: 5 to 9, 11 to 21, 25 to 29, 31 to 35, and 144 to 153 are optionally missing, and wherein L at the second last position and/or N at the last position of SEQ ID NOs: 5 to 9, 11 to 21, 25 to 29, 31 to 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁹M, wherein said ankyrin repeat domain binds human FAP-expressing WI38 cells with an EC₅₀ about or below 10⁻⁹M.

In one embodiment, said recombinant binding protein comprises an ankyrin repeat domain having binding specificity for FAP, wherein said ankyrin repeat domain binds human FAP in PBS with a dissociation constant (K_{D}) below 10⁻⁹M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 5, 9, 11 to 15, 18, 19, 26 to 29, 32 to 35, and 144 to 153, wherein G at position 1 and/or S at position 2 of SEQ ID NOs: 5, 9, 11 to 15, 18, 19, 26 to 29, 32 to 35, and 144 to 153 are optionally missing, and wherein L at the second last position and/or N at the last position of SEQ ID NOs: 5, 9, 11 to 15, 18, 19, 26 to 29, 32 to 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 5×10⁻¹⁰M, wherein said ankyrin repeat domain binds human FAP-expressing WI38 cells with an EC₅₀ about or below 10⁻⁹M.

In one embodiment, said recombinant binding protein comprises an ankyrin repeat domain having binding specificity for FAP, wherein said ankyrin repeat domain binds human FAP in PBS with a dissociation constant (K_{D}) below 5×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 5, 9, 11 to 15, 18, 19, 26 to 29, 33 to 35, and 144 to 153, wherein G at position 1 and/or S at position 2 of SEQ ID NOs: 5, 9, 11 to 15, 18, 19, 26 to 29, 33 to 35, and 144 to 153 are optionally missing, and wherein L at the second last position and/or N at the last position of SEQ ID NOs: 5, 9, 11 to 15, 18, 19, 26 to 29, 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9, 11 to 15, 18, 19, 26, 28, 29, 33 to 35, and 144 to 153, wherein G at position 1 and/or S at position 2 of SEQ ID NOs: 9, 11 to 15, 18, 19, 26, 28, 29, 33 to 35, and 144 to 153 are optionally missing, and wherein L at the second last position and/or N at the last position of SEQ ID NOs: 9, 11 to 15, 18, 19, 26, 28, 29, 33, 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9, 11 to 15, 18, 19, 26, 28, 29, and 33 to 35, wherein G at position 1 and/or S at position 2 of SEQ ID NOs: 9, 11 to 15, 18, 19, 26, 28, 29, and 33 to 35 are optionally missing, and wherein L at the second last position and/or N at the last position of SEQ ID NOs: 9, 11 to 15, 18, 19, 26, 28, 29 and 33 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or Sat position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148 to 150 are optionally exchanged by A. Thus, in one embodiment, said ankyrin repeat domain comprises an amino acid sequence with at least 90% amino acid sequence identity with any one of SEQ ID NOs: 18, 19, 26, 33 to 35, and 144 to 153. In another embodiment, said ankyrin repeat domain comprises an amino acid sequence with at least 93% amino acid sequence identity with any one of SEQ ID NOs: 18, 19, 26, 33 to 35, and 144 to 153; and in a further embodiment, said ankyrin repeat domain comprises an amino acid sequence with at least 95% amino acid sequence identity with any one of SEQ ID NOs: 18, 19, 26, 33 to 35, and 144 to 153. In one embodiment, said ankyrin repeat domain comprises an amino acid sequence with at least 98% amino acid sequence identity with any one of SEQ ID NOs: 18, 19, 26, 33 to 35, and 144 to 153; and in one embodiment, said ankyrin repeat domain comprises an amino acid sequence of any one of SEQ ID NOs: 18, 19, 26, 33 to 35, and 144 to 153.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or Sat position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A. Thus, in one embodiment, said ankyrin repeat domain comprises an amino acid sequence with at least 90% amino acid sequence identity with any one of SEQ ID NOs: 18, 19, 26, and 33 to 35. In another embodiment, said ankyrin repeat domain comprises an amino acid sequence with at least 93% amino acid sequence identity with any one of SEQ ID NOs: 18, 19, 26, and 33 to 35; and in a further embodiment, said ankyrin repeat domain comprises an amino acid sequence with at least 95% amino acid sequence identity with any one of SEQ ID NOs: 18, 19, 26, and 33 to 35. In one embodiment, said ankyrin repeat domain comprises an amino acid sequence with at least 98% amino acid sequence identity with any one of SEQ ID NOs: 18, 19, 26, and 33 to 35; and in one embodiment, said ankyrin repeat domain comprises an amino acid sequence of any one of SEQ ID NOs: 18, 19, 26, and 33 to 35.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A. Thus, in one embodiment, said ankyrin repeat domain comprises an amino acid sequence with at least 90% amino acid sequence identity with SEQ ID NO: 18. In another embodiment, said ankyrin repeat domain comprises an amino acid sequence with at least 93% amino acid sequence identity with SEQ ID NO: 18; and in a further embodiment, said ankyrin repeat domain comprises an amino acid sequence with at least 95% amino acid sequence identity with SEQ ID NO: 18. In one embodiment, said ankyrin repeat domain comprises an amino acid sequence with at least 98% amino acid sequence identity with SEQ ID NO: 18; and in one embodiment, said ankyrin repeat domain comprises the amino acid sequence of SEQ ID NO: 18.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 19, wherein G at position 1 and/or S at position 2 of SEQ ID NO: 19 are optionally missing, and wherein L at the second last position and/or N at the last position of SEQ ID NO: 19 are optionally exchanged by A. Thus, in one embodiment, said ankyrin repeat domain comprises an amino acid sequence with at least 90% amino acid sequence identity with SEQ ID NO: 19. In another embodiment, said ankyrin repeat domain comprises an amino acid sequence with at least 93% amino acid sequence identity with SEQ ID NO: 19; and in a further embodiment, said ankyrin repeat domain comprises an amino acid sequence with at least 95% amino acid sequence identity with SEQ ID NO: 19. In one embodiment, said ankyrin repeat domain comprises an amino acid sequence with at least 98% amino acid sequence identity with SEQ ID NO: 19; and in one embodiment, said ankyrin repeat domain comprises the amino acid sequence of SEQ ID NO: 19.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing. Thus, in one embodiment, said ankyrin repeat domain comprises an amino acid sequence with at least 90% amino acid sequence identity with SEQ ID NO: 34. In another embodiment, said ankyrin repeat domain comprises an amino acid sequence with at least 93% amino acid sequence identity with SEQ ID NO: 34; and in a further embodiment, said ankyrin repeat domain comprises an amino acid sequence with at least 95% amino acid sequence identity with SEQ ID NO: 34. In one embodiment, said ankyrin repeat domain comprises an amino acid sequence with at least 98% amino acid sequence identity with SEQ ID NO: 34; and in one embodiment, said ankyrin repeat domain comprises the amino acid sequence of SEQ ID NO: 34.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence with at least 90%, amino acid sequence identity with any one of SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A. Thus, in one embodiment, said ankyrin repeat domain comprises an amino acid sequence with at least 93% amino acid sequence identity with any one of SEQ ID NOs: 144 to 153; and in a further embodiment, said ankyrin repeat domain comprises an amino acid sequence with at least 95% amino acid sequence identity with any one of SEQ ID NOs: 144 to 153. In one embodiment, said ankyrin repeat domain comprises an amino acid sequence with at least 98% amino acid sequence identity with any one of SEQ ID NOs: 144 to 153; and in one embodiment, said ankyrin repeat domain comprises an amino acid sequence of any one of SEQ ID NOs: 144 to 153. Thus, in one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 10⁻⁹M, and wherein said ankyrin repeat domain comprises an amino acid sequence selected from SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 3×10⁻¹⁰M.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, wherein said ankyrin repeat domain binds human FAP-expressing WI38 cells with an EC₅₀ about or below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9, 13 to 15, 18, 19, 26 and 33 wherein G at position 1 and/or Sat position 2 of SEQ ID NOs: 9, 13 to 15, 18, 19, 26 and 33 are optionally missing, and wherein L at the second last position and/or N at the last position of SEQ ID NOs: 9, 13 to 15, 18, 19, 26 and 33 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 2×10⁻¹⁰M, wherein said ankyrin repeat domain binds human FAP-expressing WI38 cells with an EC₅₀ about or below 3×10⁻¹⁰M.

In one embodiment, said recombinant binding protein comprises an ankyrin repeat domain having binding specificity for FAP, wherein said ankyrin repeat domain binds human FAP in PBS with a dissociation constant (K_{D}) below 2×10⁻¹⁰M, wherein said ankyrin repeat domain binds human FAP-expressing WI38 cells with an EC₅₀ about or below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 9, 14, 18 and 26, wherein G at position 1 and/or S at position 2 of SEQ ID NOs: 9, 14, 18 and 26 are optionally missing, and wherein L at the second last position and/or N at the last position of SEQ ID NOs: 9, 14, 18 and 26 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, or by more than 20%, or by more than 15%, or by more than 10%, or by more than 9%, 8%, 7%, 6%, 5%, 4%, 3%, 2%, 1% or 0%. Thus, in one embodiment, said binding of said ankyrin repeat domain to FAP does not inhibit the prolyl endopeptidase activity of FAP by more than 25%. In another embodiment, said binding of said ankyrin repeat domain to FAP does not inhibit the prolyl endopeptidase activity of FAP by more than 20%; and in a further embodiment, said binding of said ankyrin repeat domain to FAP does not inhibit the prolyl endopeptidase activity of FAP by more than 15%. In one embodiment, said binding of said ankyrin repeat domain to FAP does not inhibit the prolyl endopeptidase activity of FAP by more than 10%.

A typical and preferred method to determine whether binding of the inventive recombinant binding proteins to FAP inhibits the prolyl endopeptidase activity of FAP is described in Example 6. Thus, in one embodiment said inhibition of the prolyl endopeptidase activity of FAP by the inventive recombinant binding proteins is determined as described in Example 6.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or Sat position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148 to 150 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or Sat position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 20%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or Sat position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 15%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or Sat position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 20%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 20%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain comprises an amino acid sequence with at least 90%, 91%, 92%, 93%, 94%, 95%, 96%, 97%, 98%, 99%, 100% amino acid sequence identity with any one of SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 20%, and wherein said ankyrin repeat domain comprises an amino acid sequence with at least 90%, 91%, 92%, 93%, 94%, 95%, 96%, 97%, 98%, 99%, 100% amino acid sequence identity with any one of SEQ ID NOs: 144 to 153, wherein G at position 1 and/or S at position 2 of SEQ ID NOs: 144 to 153 are optionally missing, and wherein L at the second last position and/or N at the last position of SEQ ID NOs: 144, 145 and 148 to 150 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or S at position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148 to 150 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or S at position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP in PBS with a dissociation constant (K_{D}) below 3×10⁻¹⁰M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or S at position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148 to 150 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or S at position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein binding of said ankyrin repeat domain to FAP does not inhibit the prolyl endopeptidase activity of FAP by more than 25%, and wherein said ankyrin repeat domain binds human FAP-expressing WI38 cells with an EC₅₀ about or below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, 33 to 35, and 144 to 153, wherein G at position 1 and/or Sat position 2 of SEQ ID NOs: 18, 19, 26, 33 to 35, and 144 to 153 are optionally missing, and wherein L at the second last position and/or N at the last position of SEQ ID NOs: 18, 19, 26, 33, 144, 145 and 148-150 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 18, 19, 26, and 33 to 35, wherein G at position 1 and/or S at position 2 of SEQ ID NOs: 18, 19, 26, and 33 to 35 are optionally missing, and wherein L at the second last position and/or N at the last position of SEQ ID NOs: 18, 19, 26 and 33 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 20%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 18, wherein G at position 1 and/or S at position 2 of SEQ ID NO: 18 are optionally missing, and wherein L at the second last position and/or N at the last position of SEQ ID NO: 18 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 19, wherein G at position 1 and/or S at position 2 of SEQ ID NO: 19 are optionally missing, and wherein L at the second last position and/or N at the last position of SEQ ID NO: 19 are optionally exchanged by A.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 25%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, said recombinant binding protein comprises an ankyrin repeat domain having binding specificity for FAP, wherein said ankyrin repeat domain binds human FAP in PBS with a dissociation constant (K_{D}) below 3×10⁻¹⁰M, and wherein said ankyrin repeat domain binds human FAP-expressing WI38 cells with an EC₅₀ about or below 10⁻⁹M, and wherein binding of said ankyrin repeat domain to FAP does not inhibit the prolyl endopeptidase activity of FAP by more than 20%, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 34, wherein G at position 1 and/or S at position 2 of SEQ ID NO: 34 are optionally missing.

In one embodiment, any of the sequence variability in said ankyrin repeat domain(s) described above occurs in the N-terminal capping module, the C-terminal capping module and/or the ankyrin repeat modules, wherein any sequence variability in the ankyrin repeat modules occurs only in framework positions and in positions other than the randomized positions 3, 4, 6, 14 and 15 of the ankyrin repeat modules, and wherein typically the overall structure of the ankyrin repeat modules and the ankyrin repeat domain is not affected by the sequence variability.

In one embodiment, said recombinant binding protein comprises an ankyrin repeat domain having binding specificity for FAP, wherein said binding protein further comprises a biologically active molecule.

In one embodiment and as an example, said biologically active molecule is able to increase the in vivo half-life of the recombinant protein of the present invention. In one embodiment, said biologically active molecule is an ankyrin repeat domain with binding specificity for serum albumin. In one embodiment, said biologically active molecule is an ankyrin repeat domain with binding specificity for serum albumin, wherein said ankyrin repeat domain with binding specificity for serum albumin consists of SEQ ID NO: 38 or sequences with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 38. In one embodiment, said biologically active molecule is an ankyrin repeat domain with binding specificity for serum albumin, wherein said ankyrin repeat domain with binding specificity for serum albumin consists of SEQ ID NO: 38. In one embodiment, said ankyrin repeat domain with binding specificity for serum albumin consisting of SEQ ID NO: 38 is connected to said ankyrin repeat domain having binding specificity for FAP with a peptide linker, wherein said peptide linker is preferably a proline-threonine rich peptide linker, further preferably the peptide linker of SEQ ID NO:39.

In one embodiment, said recombinant protein comprises an amino acid sequence selected from the group consisting of (1) SEQ ID NOs: 40 to 42 and (2) sequences in which up to 9 amino acids in any of SEQ ID NOs: 40 to 42 are exchanged by another amino acid. In one embodiment, said recombinant protein comprises the amino acid sequence selected from the group consisting of (1) SEQ ID NO: 40 and (2) sequences in which up to 9, 8, 7, 6, 5, 4, 3, 2, 1 or 0 amino acids in SEQ ID NO: 40 are exchanged by another amino acid. In one embodiment, said recombinant protein comprises the amino acid sequence selected from the group consisting of (1) SEQ ID NO: 41 and (2) sequences in which up to 9, 8, 7, 6, 5, 4 3, 2, 1 or 0 amino acids in SEQ ID NO: 41 are exchanged by another amino acid. In one embodiment, said recombinant protein comprises the amino acid sequence selected from the group consisting of (1) SEQ ID NO: 42 and (2) sequences in which up to 9, 8, 7, 6, 5, 4, 3, 2, 1 or 0 amino acids in SEQ ID NO: 42 are exchanged by another amino acid.

In one embodiment, said biologically active molecule is an ankyrin repeat domain with binding specificity for serum albumin, wherein said ankyrin repeat domain with binding specificity for serum albumin consists of an amino acid sequence selected from the group consisting of (1) SEQ ID NO: 159 and (2) sequences in which up to 9, 8, 7, 6, 5, 4, 3, 2, 1 or 0 amino acids in SEQ ID NO: 159 are exchanged by another amino acid. In one embodiment, said biologically active molecule is an ankyrin repeat domain with binding specificity for serum albumin, wherein said ankyrin repeat domain with binding specificity for serum albumin consists of an amino acid sequence selected from the group consisting of (1) SEQ ID NO: 160 and (2) sequences in which up to 9, 8, 7, 6, 5, 4, 3, 2, 1 or 0 amino acids in SEQ ID NO: 160 are exchanged by another amino acid.

In one embodiment, said recombinant protein comprises a first amino acid sequence selected from the group consisting of (1) SEQ ID NO:34 and (2) sequences in which up to 9, 8, 7, 6, 5, 4, 3, 2, 1 or 0 amino acids in SEQ ID NO:34 are exchanged by another amino acid, and further comprises a second amino acid sequence selected from the group consisting of (1) SEQ ID NO:159 and (2) sequences in which up to 9, 8, 7, 6, 5, 4, 3, 2, 1 or 0 amino acids in SEQ ID NO: 159 are exchanged by another amino acid. In one embodiment, said first amino acid sequence is SEQ ID NO:34 and said second amino acid sequence is SEQ ID NO:159. In one embodiment, said first amino acid sequence and said second amino acid sequence are connected with a peptide linker. In one embodiment, said peptide linker is a proline-threonine rich peptide linker, preferably the peptide linker of SEQ ID NO:39.

Importantly, it has further been shown (Example 5) that the fusion of a biologically active molecule to a FAP-specific ankyrin repeat domain does not affect the ability of the recombinant protein of the present invention to specifically recognize and bind to FAP expressed on the surface of cells. As shown in Example 5, the fusion proteins comprising the FAP-specific ankyrin repeat domains retain their ability to efficiently bind to FAP expressed on the surface of cells irrespective of the further biologically active molecule being present in the inventive recombinant proteins.

Furthermore, it has been shown (Example 5) that the recombinant proteins of the present invention comprising ankyrin repeat domains having binding specificity for FAP are able to in vivo localize or deliver or target a biologically active molecule preferentially to tumor tissue expressing FAP. This has been assessed in a FAP-positive mouse tumor model by biodistribution analysis using radioactively labeled recombinant proteins of the present invention. The 48 hours post-injection biodistribution analysis further shows that the recombinant proteins of the present invention comprising ankyrin repeat domains having binding specificity for FAP and further comprising a biologically active molecule such as ankyrin repeat domains with binding specificity for serum albumin are not only able to localize or deliver or target said biologically active molecules and recombinant proteins to FAP-expressing tumor tissues but further are able to accumulate and extend retention of said biologically active molecules in FAP-expressing tumor tissues. The recombinant proteins of the present invention comprising biologically or therapeutically active molecules are therefore able to reduce potential side effects of said biologically or therapeutically active molecules in other organs and the organism as a whole while localizing, delivering, targeting, accumulating, activating and/or retaining said inventive recombinant proteins in FAP-expressing tumors. The recombinant proteins of the present invention may serve as components of FAP-specific therapeutic, imaging and/or diagnostic agents.

Moreover, it has been shown (Example 6) that the recombinant proteins of the present invention comprising ankyrin repeat domains having binding specificity for FAP do not significantly inhibit FAP enzymatic activity. This has been assessed by measuring the prolyl endopeptidase activity of FAP on a fluorogenic substrate in the presence and in the absence of recombinant proteins of the present invention.

FAP-specific ankyrin repeat domains of the invention can serve as building blocks for binding proteins comprising one or more additional ankyrin repeat domains. Multiple features characterize the proteins encoded by SEQ ID NOs: 40-42 as preferred recombinant binding proteins of the invention. They comprise a designed ankyrin repeat domain with binding specificity for FAP and a designed ankyrin repeat domain with binding specificity for serum albumin. They are the first recombinant binding proteins combining serum albumin-binding and FAP-binding. Ankyrin repeat domains with binding specificity for serum albumin, in particular the ankyrin repeat domain of SEQ ID NO: 38, SEQ ID NO: 159 or SEQ ID NO: 160, or variants thereof, lead to improved in vivo half-lives. As a consequence, recombinant proteins comprising an ankyrin repeat domain having binding specificity for FAP and comprising an ankyrin repeat domain having binding specificity for serum albumin are embodiments particularly useful for therapeutic applications.

In one embodiment, said ankyrin repeat domain with binding specificity for serum albumin binds serum albumin of mouse, rat, dog, cynomolgus monkey, or human origin, more preferably serum albumin of mouse, cynomolgus monkey or human origin, more preferably serum albumin of cynomolgus monkey or human origin, more preferably serum albumin of human origin, in PBS with a dissociation constant (K_{D}) below 10⁻⁵M; preferably below 10⁻⁶M; or more preferably below 10⁻⁷M. In one embodiment, said designed ankyrin repeat domain with binding specificity for serum albumin consists of SEQ ID NO: 38, SEQ ID NO: 159 or SEQ ID NO: 160 and binds human serum albumin in PBS with a dissociation constant (K_{D}) below 10⁻⁵M; preferably below 10⁻⁶M; or more preferably below 10⁻⁷M. Examples of dissociation constant determination using surface plasmon resonance are given in the Examples, e.g. Example 2, and in WO2014/083208.

In one embodiment, said FAP-specific recombinant binding protein of the invention further comprises a polypeptide tag. A polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a binding protein may be connected to other parts of the binding protein directly or via peptide linkers. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one embodiment, said FAP-specific recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO: 155, and an example of a proline-threonine-linker is the amino acid sequence of SEQ ID NO:39.

In another aspect, the invention relates to a nucleic acid encoding the amino acid sequence of an ankyrin repeat domain or a recombinant binding protein of the present invention. In one embodiment, the invention relates to a nucleic acid encoding the amino acid sequence of a recombinant binding protein of the present invention. In one embodiment, the invention relates to a nucleic acid encoding an amino acid sequence selected from the group consisting of SEQ ID NO: 18, SEQ ID NO: 19, SEQ ID NO: 26, SEQ ID NO: 33, SEQ ID NO: 34 and SEQ ID NO: 35. In one embodiment, the invention relates to a nucleic acid encoding the amino acid sequence of SEQ ID NO: 18. In one embodiment, the invention relates to a nucleic acid encoding the amino acid sequence of SEQ ID NO: 19. In one embodiment, the invention relates to a nucleic acid encoding the amino acid sequence of SEQ ID NO: 34. In one embodiment, the invention relates to a nucleic acid encoding the amino acid sequence of SEQ ID NO: 35. In one embodiment, the invention relates to a nucleic acid encoding the recombinant binding protein consisting of SEQ ID NO: 18. In one embodiment, the invention relates to a nucleic acid encoding the recombinant binding protein consisting of SEQ ID NO: 34. Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli. An example of a nucleic acid of the invention is provided by SEQ ID NO: 156, which encodes the amino acid sequence of SEQ ID NO:34.

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present invention, and optionally a pharmaceutically acceptable carrier and/or diluent.

In one embodiment, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below. Even further, a diagnostic composition or a tumor imaging composition is provided comprising one or more of the above mentioned recombinant binding proteins and/or designed ankyrin repeat domains, and/or nucleic acids, in particular recombinant binding proteins and/or nucleic acids of the present invention.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient or stabilizer, for example as described in Remington's Pharmaceutical Sciences 16th edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

One embodiment of the present invention relates to the use of a recombinant binding protein of the present invention comprising an ankyrin repeat domain having binding specificity for FAP and further comprising a biologically active molecule such as an ankyrin repeat domain with binding specificity for serum albumin for manufacturing a pharmaceutical composition, wherein said recombinant binding protein exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for FAP but not said ankyrin repeat domain with binding specificity for serum albumin. In one embodiment of the invention, a recombinant binding protein comprises an ankyrin repeat domain having binding specificity for FAP and further comprises two ankyrin repeat domains with binding specificity for serum albumin.

In one embodiment, a pharmaceutical composition comprises at least one recombinant binding protein as described herein and a detergent such as nonionic detergent, a buffer such as phosphate buffer, and a sugar such as sucrose. In one embodiment, such a composition comprises recombinant binding proteins as described above and PBS.

In another aspect, the invention provides a method of localizing a biologically active molecule to FAP-expressing cells or tissue in a mammal, the method comprising the step of administering to said mammal the inventive recombinant binding protein comprising said biologically active molecule. In one embodiment, said biologically active molecule is a binding protein having binding specificity for a target different from FAP. In one embodiment, said mammal is a human and said FAP-expressing cells or tissue are located in a tumor, including in a primary tumor, metastasis and/or tumor stroma. Selective localization, accumulation, retention and/or activation of a biologically active molecule in tumor tissue have the advantage of concentrating the activity of the molecule in the tumor tissue, while resulting in much less activity of the molecule in normal and non-tumorigenic tissues. Such effects may be independent of FAP's enzymatic activity or role in tumor progression, relying on FAP's localization within the tumor microenvironment.

In another aspect, the invention provides a method of treating a medical condition, the method comprising the step of administering to a patient in need thereof a therapeutically effective amount of the inventive recombinant binding protein comprising a biologically active molecule, wherein said biologically active molecule is a therapeutically effective molecule. In one embodiment, said biologically active molecule is a therapeutically effective molecule when localized to FAP-expressing cells or tissue. In one embodiment, said biologically active molecule is a binding protein having binding specificity for a target different from FAP. In one embodiment, said FAP-expressing cells or tissue are located in a tumor, including in a primary tumor, metastasis and/or tumor stroma. These embodiments thus allow to take advantage of FAP's restricted expression in tumor stroma by localizing the activity of a biologically active molecule, such as, e.g. an immune checkpoint inhibitor or an immune co-stimulatory agonist, to the tumor microenvironment.

In another aspect, the invention provides a method of imaging a tumor in a patient, the method comprising the step of administering to a patient in need thereof the inventive recombinant binding protein comprising a biologically active molecule, wherein said biologically active molecule is a molecule effective for imaging cells bound by said binding protein.

In another aspect, the invention provides a method of diagnosing a cancer in a patient, the method comprising the step of administering to a patient in need thereof the inventive recombinant binding protein comprising a biologically active molecule, wherein said biologically active molecule is a molecule effective for diagnosing the cancer of said patient.

In one embodiment, the invention relates to the use of a pharmaceutical composition, or a recombinant binding protein according to the present invention for the treatment of a disease. For that purpose, the pharmaceutical composition, or the recombinant binding protein according to the present invention is administered, to a patient in need thereof, in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above mentioned pharmaceutical composition or recombinant binding protein is considered for the treatment of a disorder.

In one embodiment, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one embodiment, the invention relates to a method of treatment of a medical condition, the method comprising the step of administering, to a patient in need of such a treatment, a therapeutically effective amount of a recombinant binding protein of the invention. In one embodiment, the invention relates to a method of treatment of a medical condition, the method comprising the step of administering, to a patient in need of such a treatment, a therapeutically effective amount of a pharmaceutical composition of the invention. In one embodiment, the invention relates to the use of a pharmaceutical composition of the present invention for the treatment of a disease. In one embodiment, the invention relates to a pharmaceutical composition for use in the treatment of a disease. In one embodiment, the invention relates to a pharmaceutical composition for use in the treatment of a medical condition. In one embodiment, the invention relates to a nucleic acid for use in the treatment of a disease. In one embodiment, the invention relates to the use of said pharmaceutical composition, recombinant binding protein, or nucleic acid molecule, as medicament for the treatment of a disease. In one embodiment, the invention relates to the use of said pharmaceutical composition, recombinant binding protein, or nucleic acid molecule, for manufacturing of a medicament. In one embodiment, the invention relates to the use of said pharmaceutical composition, recombinant binding protein, or nucleic acid molecule, for manufacturing of a medicament for the treatment of a disease. In one embodiment, the invention relates to a process for the manufacturing of a medicament for the treatment of a disease, wherein said pharmaceutical composition, recombinant binding protein, or nucleic acid molecule is active ingredient of the medicament. In one embodiment, the invention relates to a process of treatment of a disease using said pharmaceutical composition, recombinant binding protein, or nucleic acid molecule.

In particular, the invention relates to the treatment of a medical condition using a pharmaceutical composition of the present invention, wherein said medical condition is cancer.

The use of a recombinant binding protein of the present invention or said pharmaceutical compositions for the treatment of cancer diseases can also be in combination with one or more other therapies known in the art. The term “use in combination with”, as used herein, shall refer to a co-administration, which is carried out under a given regimen. This includes synchronous administration of the different compounds as well as time-shifted administration of the different compounds (e.g. compound A is given once and compound B is given several times thereafter, or vice versa, or both compounds are given synchronously and one of the two is also given at later stages).

In a further embodiment, the invention relates to the use of a recombinant binding protein of the invention for the manufacture of a medicament that is used for the treatment of a medical condition, preferably a neoplastic disease, more preferably cancer.

In one embodiment, the invention relates to the use of a pharmaceutical composition of the invention for the manufacture of a medicament that is used for the treatment of a medical condition, which may be a neoplastic disease, in particular cancer.

In one embodiment the invention relates to a recombinant binding protein comprising any of the above mentioned ankyrin repeat domains.

In one embodiment, the invention relates to a kit comprising said recombinant binding protein. In one embodiment, the invention relates to a kit comprising a nucleic acid encoding said recombinant binding protein. In one embodiment, the invention relates to a kit comprising said pharmaceutical composition. In one embodiment, the invention relates to a kit comprising said recombinant binding protein, and/or a nucleic acid encoding said recombinant binding protein, and/or said pharmaceutical composition. In one embodiment, the invention relates to a kit comprising the recombinant binding protein comprising a FAP-specific ankyrin repeat domain, for example SEQ ID NO:18 or SEQ ID NO: 34, and/or a nucleic acid encoding the recombinant binding protein comprising a FAP-specific ankyrin repeat domain, for example SEQ ID NO:18 or SEQ ID NO: 34, and/or a pharmaceutical composition comprising the recombinant binding protein comprising a FAP-specific ankyrin repeat domain, for example SEQ ID NO:18 or SEQ ID NO: 34, and/or a nucleic acid encoding the recombinant binding protein comprising a FAP-specific ankyrin repeat domain, for example SEQ ID NO:18 or SEQ ID NO: 34.

In one embodiment, the invention relates to a method for producing a recombinant binding protein of the present invention. In one embodiment, the invention relates to a method for producing a recombinant binding protein, for example a recombinant binding protein comprising the amino acid sequence of SEQ ID NO:18 or SEQ ID NO:34, the method comprising the steps of (i) expressing said recombinant binding protein in bacteria, and (ii) purifying said recombinant binding protein using chromatography. Said method may comprise additional steps. Such a method of producing a recombinant binding protein of the present invention is described in Example 1.

The invention is not restricted to the particular embodiments described in the Examples.

This specification refers to a number of amino acid sequences of the amino acid sequence listing of this specification named “P5618_Sequence_Listing.txt” and the amino acid sequences of the sequence listing are herewith incorporated by reference.

### Definitions

Unless defined otherwise herein, all technical and scientific terms used herein shall have the meanings that are commonly understood by those of ordinary skill in the art to which the present invention belongs.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for FAP.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins (e.g. DM1 from Immunogen), small molecules, antibiotics and alike) well known to the person skilled in the art. A binding protein of the instant invention may comprise a localizer molecule.

The term “binding domain” means a protein domain exhibiting binding specificity for a target. Preferably, said binding domain is a recombinant binding domain.

The term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, FAP and FAP-expressing cells and tissues are targets of FAP-specific binding proteins.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11 (5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain.

The term “target interaction residues” refers to amino acid residues of a repeat module, which contribute to the direct interaction with a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target, e.g. as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 14 and 15 of SEQ ID NOs: 48 to 134 are randomized positions of the ankyrin repeat modules of the instant invention.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “has binding specificity for a target”, “specifically binding to a target”, “binding to a target with high specificity”, “specific for a target” or “target specificity” and the like means that a binding protein or binding domain binds in PBS to a target with a lower dissociation constant (i.e. it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the inventive ankyrin repeat domains and recombinant binding proteins with binding specificity for FAP by Surface Plasmon Resonance (SPR) analysis is described in Example 2.

The term “about” means the mentioned value+/−20%; for example “about 50” shall mean 40 to 60.

The term “PBS” means a phosphate buffered water solution containing 137 mM NaCl, 10 mM phosphate and 2.7 mM KCl and having a pH of 7.4.

The term “mouse serum albumin” refers to UniProt accession number P07724, the term “cynomolgus monkey serum albumin” (i.e. Macaca fascicularis) refers to UniProt accession number A2V9Z4, and the term “human serum albumin” refers to UniProt accession number P02768. The amino acid sequence of human serum albumin is provided in SEQ ID NO: 154.

Preferably, clearance, and/or exposure, and/or terminal half-life are assessed in a mammal, more preferably mouse and/or cynomolgus monkey, more preferably cynomolgus monkey. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art. Preferably, pharmacokinetic comparison is done at any dose, more preferably at equivalent dose (i.e. same mg/kg dose) or equimolar dose (i.e. same mol/kg dose), more preferably at equimolar dose (i.e. same mol/kg dose). It is understood by the person skilled in the art that equivalent and/or equimolar dosing in animals is subject to experimental dose variations of at least 20%, more preferably 30%, 40%, 50%, 60%, 70%, 80%, 90%, or 100%. Preferably, a dose used for pharmacokinetic measurement is selected from 0.001 to 1000 mg/kg, more preferably 0.01 to 100 mg/kg, more preferably 0.1 to 50 mg/kg, more preferably 0.5 to 10 mg/kg.

The term “fibroblast activation protein” or “FAP”, also known as Prolyl endopeptidase FAP or Seprase (EC 3.4.21), refers to any native FAP from any vertebrate source, including mammals such as primates (e.g. humans and non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. An amino acid sequence of human FAP is shown in UniProt (www.uniprot.org) accession no. Q12884, or NCBI (www.ncbi.nlm.nih.gov/) Ref. Seq. NP_004451.2. The extracellular domain (ECD) of human FAP extends from amino acid position 26 to 760. The amino acid sequence of a His-tagged human FAP ECD is provided in SEQ ID NO: 45. An amino acid sequence of mouse FAP is shown in UniProt accession no. P97321, or NCBI Ref. Seq. NP_032012.1. The extracellular domain (ECD) of mouse FAP extends from amino acid position 26 to 761. SEQ ID NO: 47 shows the amino acid sequence of mouse FAP ECD. An amino acid sequence of cynomolgus FAP is shown in NCBI Ref. Seq. XP_005573377.1. SEQ ID NO: 46 shows the amino acid sequence of a His-tagged cynomolgus FAP ECD. Preferably, an anti-FAP binding molecule of the invention binds to the extracellular domain of FAP.

The terms “bioactive molecule” and “biologically active molecule” are intended to encompass any molecule that has a biological effect or activity in a mammal, including, e.g., a human, and that can be covalently or non-covalently linked, conjugated, fused or otherwise physically associated with a binding protein of the invention. The terms encompass also any such molecule that exhibits the biological effect or activity only upon an activation event, such as, e.g., clustering, proteolytic cleavage, allosteric change or dimerization. The terms encompass polynucleotides, peptides/polypeptides and/or pharmaceutical agents. The term “polynucleotides” generally refers to DNA unless otherwise indicated, but may include RNA and modified or artificial forms of DNA or RNA. The term includes genes, cDNA, oligonucleotides, RNAi, plasmids etc. The term further includes sense DNA or RNA for expressing a product in a target organ, and antisense DNA or RNA for reducing or eliminating expression of a native or introduced gene in a target organ. The term “peptide” refers to a peptide chain of 4 to 600 amino acids long, such as 4 to 200 amino acids long, and therefore encompasses polypeptides and proteins. The term encompasses any naturally occurring or man-made binding proteins, binding domains, growth factor receptors or fragments or ligands thereof, cytokines, enzymes, polypeptide hormones, antibodies, antibody-like proteins based on scaffolds, immunomodulatory proteins, etc. Furthermore, the term “peptide” also encompasses peptides modified by, e.g, glycosylation, and proteins comprising two or more polypeptide chains, each of length of 4 to 600 amino acids long, cross-linked by, e.g., disulphide bonds, such as, e.g., insulin and immunoglobulins. The term “pharmaceutical agent” is intended to include any natural or synthetic compound that may be administered to a recipient in order to induce a physiological, pharmacological or therapeutic effect. Examples of such agents are anti-tumor drugs, toxins, antibiotics, hormones, anti-inflammatory agents, anti-parasitic agents, DNA vaccines, etc.

The term “antibody” means not only intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)2, and single chain V region fragments (scFv), bispecific antibodies, chimeric antibodies, antibody fusion polypeptides, and unconventional antibodies.

The term “localizing” or “delivering” as interchangeably used herein in the context of “localizing a biologically active molecule to FAP-expressing cells or tissue” refers to increased localization of a biologically active molecule to FAP-expressing cells or tissue in a mammal when the biologically active molecule is linked to a FAP-specific binding protein as compared to when the biologically active molecule is not linked to a FAP-specific binding protein. The term also refers to targeting a molecule to the site of a target in a mammal, wherein the molecule is a biologically active molecule linked to a FAP-specific ankyrin repeat domain of the invention and wherein the target is FAP and wherein the site of the target is FAP-expressing cells or tissue. The term preferably further encompasses the accumulation and/or retention of a biologically active molecule linked to a FAP-specific ankyrin repeat domain of the invention at the site of FAP-expressing cells or tissue in a mammal. The term also preferably encompasses the localized activation of a biologically active molecule linked to a FAP-specific ankyrin repeat domain of the invention or of a biological response induced by the biologically active molecule at the site of FAP-expressing cells or tissue in a mammal. Such localized activation may occur, for example, through clustering of the biologically active molecule or of a protein (such as, e.g., a cell surface receptor) bound by the biologically active molecule upon binding of the linked ankyrin repeat domain of the invention to FAP-expressing cells or tissue. Such localized activation may also occur through other mechanisms, for example, through proteolytic cleavage, allosteric change or dimerization of the biologically active molecule (e.g. as in the activation of a prodrug). As used in this paragraph, “mammal” encompasses human. The result of “localizing” may be measured by various means well known to one of skill in the art. As an example, “localizing” may be measured by determining the organ-to-blood ratio of a biologically active molecule linked to an ankyrin repeat domain of the invention, as described in Example 5. In one embodiment of the invention, the effect of a FAP-specific ankyrin repeat domain on “localizing” a linked biologically active molecule is exhibited by an increased organ-to-blood ratio of at least 5%, 10%, 15%, 20%, 25%, 30%, 35%, 40%, 45%, 50%, 60%, 70%, 80%, 90%, 100%, 150%, 200%, 250%, or 300% compared to a corresponding biologically active molecule that is not linked to FAP-specific ankyrin repeat domain.

The term “FAP-expressing cells” or “FAP-expressing tissue”, as used herein, refers to cells, or tissue comprising cells, expressing FAP. Such FAP-expressing cells include fibroblasts, and in particular tumor stromal fibroblasts. FAP is expressed selectively in stromal fibroblasts of more than 90% of epithelial malignancies (primary and metastatic), including lung, colorectal, bladder, ovarian and breast carcinomas, and in malignant mesenchymal cells of bone and soft tissue sarcomas, while it is generally absent from normal adult tissues. FAP is also expressed on certain malignant tumor cells such as skin, prostate and pancreatic tumor cells (Rettig et al., Proc Natl Acad Sci USA 85, 3110-3114 (1988); Garin-Chesa et al., Proc Natl Acad Sci USA 87, 7235-7239 (1990); Rettig et al., Cancer Res. 53:3327-3335 (1993); Jin et al., Anticancer Res 23, 3195-3198 (2003); Brennen et al., Mol Cancer Ther. 11: 257-266 (2012); Hamson et al., Proteomics Clin Appl. 8: 454-63 (2014)).

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, and a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is a cancer. The term “therapeutically effective amount” means an amount that is sufficient to produce a desired effect on a patient.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

### Examples

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). The cloning and protein production strain was E. coli XL1-blue (Stratagene, USA) or BL21 (Novagen, USA). Recombinant human FAP was purchased either at RnD Systems (USA, product number 3715-SE) or at Proteros (Germany, product number PR-0071), recombinant mouse FAP was from RnD Systems (USA, product number 8647-SE). Histidine-tagged cynomolgus FAP was expressed at ReliaTech (Germany) in insect cells and was purified at Molecular Partners using standard purification methods. Biotinylated FAP was obtained chemically via coupling of a biotin moiety to primary amines of the protein using standard biotinylation reagents and methods. The Z-Gly-Pro-AMC fluorogenic substrate for the human FAP activity assay was from Bachem (product number #1-1145.0250).

Healthy female BALB/c mice for pharmacokinetic studies were supplied by Janvier, Saint Berthevin Cedex, France. DARPin® proteins were formulated in PBS solution (Gibco Life Technologies, Grand Island, N.Y., USA, Ref.: 10010-015). DARPin® protein concentrations in mouse serum samples were measured by sandwich ELISA methods using polyclonal goat anti-rabbit IgG antibody (Ab18, Thermo Scientific, No. 31210) and rabbit anti-DARPin® 1-1-1 antibody as capture reagents on NUNC Maxisorb ELISA plates. Detection was performed with murine anti-RGS-His-HRP IgG (Ab06, Qiagen, No. 34450) and TMB substrate solution.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit.. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 135, 136 or 137) or a randomized N-terminal capping module according to SEQ ID NO: 138, one or more randomized repeat modules according to the sequence motif of SEQ ID NO: 139, 140 or 141, and a fixed C-terminal capping module (e.g. the C-terminal capping module of SEQ ID NO: 142, 157 or 158) or a randomized C-terminal capping module according to SEQ ID NO: 143. Preferably, such libraries are assembled to not have any of the amino acids C, G, M, N (in front of a G residue) and P at randomized positions of repeat or capping modules. In addition, randomized repeat modules according to the sequence motif of SEQ ID NO: 139, 140 or 141 could be further randomized at position 10 and/or position 17; the randomized N-terminal capping module according to the sequence motif of SEQ ID NO: 138 could be further randomized at position 7 and/or position 9; and the randomized C-terminal capping modules according to the sequence motif of SEQ ID NO: 143 could be further randomized at positions 10, 11 and/or 17.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RILLAA, RILLKA or RELLKA motif (e.g. present from position 21 to 26 in SEQ ID NO:34) and any such C-terminal capping module of an ankyrin repeat protein library preferably possesses the KLN, KLA or KAA motif (e.g. present at the last three amino acids in SEQ ID NO:34).

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of a ankyrin repeat module of the current disclosure and consequently position 33 of a ankyrin repeat module of Binz et al. 2004, loc. cit. corresponds to position 1 of a following ankyrin repeat module of the current disclosure.

All the DNA sequences were confirmed by sequencing, and the calculated molecular weight of selected proteins was confirmed by mass spectrometry.

### Example 1: Selection of Binding Proteins Comprising an Ankyrin Repeat Domain with Binding Specificity for FAP

Using ribosome display (Hanes, J. and Plückthun, A., PNAS 94, 4937-42, 1997), many ankyrin repeat proteins with binding specificity for human FAP (hFAP) were selected from DARPin® libraries similar as described by Binz et al. 2004 (loc. cit.). The binding of the selected clones toward recombinant human FAP target was assessed by crude extract Homogeneous Time Resolved Fluorescence (HTRF), indicating that hundreds of hFAP-specific binding proteins were successfully selected. For example, the ankyrin repeat domains of SEQ ID NO: 1 to 33 constitute amino acid sequences of selected binding proteins comprising an ankyrin repeat domain with binding specificity for hFAP. Individual ankyrin repeat modules from such ankyrin repeat domains with binding specificity to hFAP are provided in SEQ ID NO: 48 to 134.

### Selection of FAP-Specific Ankyrin Repeat Proteins by Ribosome Display

The selection of hFAP-specific ankyrin repeat proteins was performed by ribosome display (Hanes and Plückthun, loc. cit.) using human FAP as target protein, libraries of ankyrin repeat proteins as described above and established protocols (Zahnd, C., Amstutz, P. and Plückthun, A., Nat. Methods 4, 69-79, 2007). The number of reverse transcription (RT)-PCR cycles after each selection round was constantly reduced from 45 to 28, adjusting to the yield due to enrichment of binders. The first four rounds of selection employed standard ribosome display selection, using decreasing target concentration and increasing washing stringency to increase selection pressure from round 1 to round 4 (Binz et al. 2004, loc. cit.). To enrich high affinity FAP-specific ankyrin repeat proteins, the output from the fourth round of standard ribosome display selection (above) was subjected to one or two off-rate selection rounds with increased selection stringency (Zahnd, 2007, loc. cit.). A final standard selection round was performed after each off-rate selection round to amplify and recover the off-rate selected binding proteins.

### Selected Clones Bind Specifically to FAP as Shown by Crude Extract HTRF

Individual selected ankyrin repeat proteins specifically binding FAP in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into the pQE30 (Qiagen) expression vector, transformed into E. coli XL1-Blue (Stratagene) and then grown overnight at 37° C. in a 96 well plate (each clone in a single well) containing 150 μl growth medium (TB containing 1% glucose and 50 μg/ml ampicillin). 150 μl of fresh LB medium containing 50 μg/ml ampicillin was inoculated with 10 μl of the overnight culture in a fresh 96-deep-well plate. After incubation for 120 minutes at 37° C. and 800 rpm, expression was induced with IPTG (0.5 mM final concentration) and continued for 4 hours. Cells were harvested, resuspended in 8.5 μl μl B-PERII (Pierce) and incubated for one hour at room temperature with shaking until complete pellet resuspension. Then, 160 μl PBS was added and cell debris were removed by centrifugation.

The extract of each lysed clone was applied as a 1:1000 dilution (final concentration) in PBSTB (PBS supplemented with 0.1% Tween 20® and 0.2% (w/v) BSA, pH 7.4) together with 2.25 nM (final concentration) biotinylated human FAP, 1:250 (final concentration) of anti-HA-D2 HTRF antibody—FRET acceptor conjugate (Cisbio) and 1:400 (final concentration) of anti-strep-Tb antibody FRET donor conjugate (Cisbio) to a well of 384 well plate and incubated for 60 minutes at RT. The HTRF was read-out on a Tecan M1000 using a 340 nm excitation wavelength and a 665±10 nm emission filter. The signal was normalized over the background. Screening of several hundred clones by such a crude cell extract HTRF revealed more than hundred different ankyrin repeat domains with specificity for human FAP. Examples of amino acid sequences of selected ankyrin repeat domains that specifically bind to human FAP are provided in SEQ ID NO: 1 to 33.

These ankyrin repeat domains with binding specificity for human FAP and a negative control ankyrin repeat domain (SEQ ID NO:44) with no binding specificity for human FAP were cloned into a pQE (QIAgen, Germany) based expression vector providing either an N-terminal His-tag (SEQ ID NO:36) or an N-terminal His-HA-tag (SEQ ID NO:37) to facilitate simple protein purification as described below. The N-terminal His-HA-tag combines a His-tag and a HA-tag. For example, expression vectors encoding the following ankyrin repeat proteins were constructed:

DARPin® protein #1 (SEQ ID NO:1 with a His-HA-tag (SEQ ID NO:37) fused to its N terminus);

DARPin® protein #2 (SEQ ID NO:2 with a His-HA-tag (SEQ ID NO:37) fused to its N terminus);

DARPin® protein #3 (SEQ ID NO:3 with a His-HA-tag (SEQ ID NO:37) fused to its N terminus);

DARPin® protein #4 (SEQ ID NO:4 with a His-HA-tag (SEQ ID NO:37) fused to its N terminus);

DARPin® protein #5 (SEQ ID NO:5 with a His-HA-tag (SEQ ID NO:37) fused to its N terminus);

DARPin® protein #6 (SEQ ID NO:6 with a His-HA-tag (SEQ ID NO:37) fused to its N terminus);

DARPin® protein #7 (SEQ ID NO:7 with a His-HA-tag (SEQ ID NO:37) fused to its N terminus);

DARPin® protein #8 (SEQ ID NO:8 with a His-HA-tag (SEQ ID NO:37) fused to its N terminus);

DARPin® protein #9 (SEQ ID NO:9 with a His-HA-tag (SEQ ID NO:37) fused to its N terminus);

DARPin® protein #10 (SEQ ID NO:10 with a His-HA-tag (SEQ ID NO:37) fused to its N terminus);

DARPin® protein #11 (SEQ ID NO:11 with a His-HA-tag (SEQ ID NO:37) fused to its N terminus);

DARPin® protein #12 (SEQ ID NO:12 with a His-HA-tag (SEQ ID NO:37) fused to its N terminus);

DARPin® protein #13 (SEQ ID NO:13 with a His-HA-tag (SEQ ID NO:37) fused to its N terminus);

DARPin® protein #14 (SEQ ID NO:14 with a His-HA-tag (SEQ ID NO:37) fused to its N terminus);

DARPin® protein #15 (SEQ ID NO:15 with a His-HA-tag (SEQ ID NO:37) fused to its N terminus);

DARPin® protein #16 (SEQ ID NO:16 with a His-HA-tag (SEQ ID NO:37) fused to its N terminus);

DARPin® protein #17 (SEQ ID NO:17 with a His-HA-tag (SEQ ID NO:37) fused to its N terminus);

DARPin® protein #18 (SEQ ID NO:18 with a His-HA-tag (SEQ ID NO:37) fused to its N terminus);

DARPin® protein #19 (SEQ ID NO:19 with a His-HA-tag (SEQ ID NO:37) fused to its N terminus);

DARPin® protein #20 (SEQ ID NO:20 with a His-HA-tag (SEQ ID NO:37) fused to its N terminus);

DARPin® protein #21 (SEQ ID NO:21 with a His-HA-tag (SEQ ID NO:37) fused to its N terminus);

DARPin® protein #22 (SEQ ID NO:22 with a His-HA-tag (SEQ ID NO:37) fused to its N terminus);

DARPin® protein #23 (SEQ ID NO:23 with a His-HA-tag (SEQ ID NO:37) fused to its N terminus);

DARPin® protein #24 (SEQ ID NO:24 with a His-HA-tag (SEQ ID NO:37) fused to its N terminus);

DARPin® protein #25 (SEQ ID NO:25 with a His-HA-tag (SEQ ID NO:37) fused to its N terminus);

DARPin® protein #26 (SEQ ID NO:26 with a His-HA-tag (SEQ ID NO:37) fused to its N terminus);

DARPin® protein #27 (SEQ ID NO:27 with a His-HA-tag (SEQ ID NO:37) fused to its N terminus);

DARPin® protein #28 (SEQ ID NO:28 with a His-HA-tag (SEQ ID NO:37) fused to its N terminus);

DARPin® protein #29 (SEQ ID NO:29 with a His-HA-tag (SEQ ID NO:37) fused to its N terminus);

DARPin® protein #30 (SEQ ID NO:30 with a His-HA-tag (SEQ ID NO:37) fused to its N terminus);

DARPin® protein #31 (SEQ ID NO:31 with a His-HA-tag (SEQ ID NO:37) fused to its N terminus);

DARPin® protein #32 (SEQ ID NO:32 with a His-HA-tag (SEQ ID NO:37) fused to its N terminus);

DARPin® protein #33 (SEQ ID NO:33 with a His-HA-tag (SEQ ID NO:37) fused to its N terminus;

DARPin® protein #36 (SEQ ID NO:18 with a His-tag (SEQ ID NO:36) fused to its N terminus);

DARPin® protein #37 (SEQ ID NO:19 with a His-tag (SEQ ID NO:36) fused to its N terminus);

DARPin® protein #38 (SEQ ID NO:26 with a His-tag (SEQ ID NO:36) fused to its N terminus);

DARPin® protein #39 (SEQ ID NO:33 with a His-tag (SEQ ID NO:36) fused to its N terminus); and

DARPin® protein #44 (SEQ ID NO:44 with a His-tag (SEQ ID NO:36) fused to its N-terminus).

### Engineering of DARPin® Protein #34 and DARPin® Protein #35

Furthermore, pQE based expression vectors were constructed encoding the following ankyrin repeat proteins that specifically bind to human FAP:

DARPin® protein #34 (SEQ ID NO:34 with a His-tag (SEQ ID NO:36) fused to its N terminus); and

DARPin® protein #35 (SEQ ID NO:35 with a His-tag (SEQ ID NO:36) fused to its N terminus). SEQ ID NO:34 and SEQ ID NO:35 were engineered based on the sequences of SEQ ID NO:18 and SEQ ID NO:19, respectively. In the ankyrin repeat domains of SEQ ID NO:18 and SEQ ID NO:19, the RILLAA motif (positions 21 to 26) present in the N-terminal capping module was replaced by the RELLKA motif and the C-terminal capping module was modified to contain the KAA motif instead of the KLN motif (positions 157 to 159 in SEQ ID NO:18 and positions 124 to 126 in SEQ ID NO:19).

### Engineering of DARPin® Proteins #144 to #153

Furthermore, pQE based expression vectors are constructed encoding the following ankyrin repeat proteins that specifically bind to human FAP:

DARPin® protein #144 (SEQ ID NO:144 with a His-tag (SEQ ID NO:36) fused to its N terminus); DARPin® protein #145 (SEQ ID NO:145 with a His-tag (SEQ ID NO:36) fused to its N terminus); DARPin® protein #146 (SEQ ID NO:146 with a His-tag (SEQ ID NO:36) fused to its N terminus); DARPin® protein #147 (SEQ ID NO:147 with a His-tag (SEQ ID NO:36) fused to its N terminus); DARPin® protein #148 (SEQ ID NO:148 with a His-tag (SEQ ID NO:36) fused to its N terminus); DARPin® protein #149 (SEQ ID NO:149 with a His-tag (SEQ ID NO:36) fused to its N terminus); DARPin® protein #150 (SEQ ID NO:150 with a His-tag (SEQ ID NO:36) fused to its N terminus); DARPin® protein #151 (SEQ ID NO:151 with a His-tag (SEQ ID NO:36) fused to its N terminus); DARPin® protein #152 (SEQ ID NO:152 with a His-tag (SEQ ID NO:36) fused to its N terminus); and DARPin® protein #153 (SEQ ID NO:153 with a His-tag (SEQ ID NO:36) fused to its N terminus).

SEQ ID NO:144 to SEQ ID NO:153 are engineered based on the sequence of SEQ ID NO:18. In the ankyrin repeat domain of SEQ ID NO:18, the RILLAA motif (positions 21 to 26) present in the N-terminal capping module is replaced by the RILLKA or RELLKA motif, and/or the C-terminal capping module is modified to contain the KAA motif instead of the KLN motif (positions 157 to 159 in SEQ ID NO:18), and/or the third repeat module is modified to replace a Proline with a Leucine (position 116 in SEQ ID NO:18). None of these modifications, alone or in combination, results in a significantly altered structure or functional properties of SEQ ID NO:144 to 153 as compared to SEQ ID NO:18.

### High Level and Soluble Expression of FAP-Specific Ankyrin Repeat Proteins

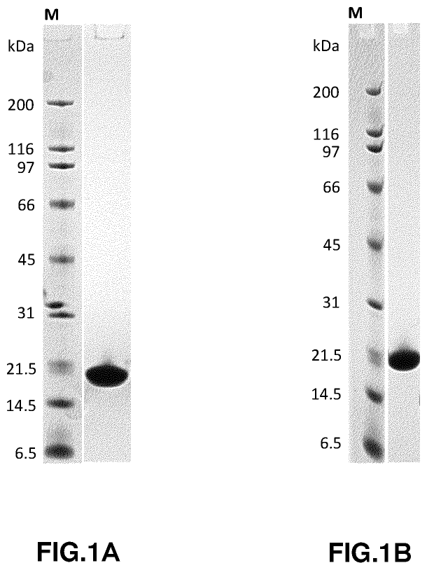

For further analysis, the selected clones showing specific FAP binding in the crude cell extract HTRF as described above were expressed in E. coli BL21 or XL1-Blue cells and purified using their His-tag according to standard protocols. 25 ml of stationary overnight cultures (TB, 1% glucose, 50 mg/I of ampicillin; 37° C.) were used to inoculate 500 ml cultures (TB, 50 mg/I ampicillin, 37° C.). At an absorbance of 1.0 to 1.5 at 600 nm, the cultures were induced with 0.5 mM IPTG and incubated at 37° C. for 4-5 h while shaking. The cultures were centrifuged and the resulting pellets were re-suspended in 25 ml of TBS₅₀₀ (50 mM Tris-HCl, 500 mM NaCl, pH 8) and lysed (sonication or French press). Following the lysis, the sample was heat-treated for 30 minutes at 62.5° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art. Alternatively, selected ankyrin repeat domains devoid of a His-tag were purified by anion exchange chromatography followed by size exclusion chromatography according to standard resins and protocols known to the person skilled in the art. Highly soluble ankyrin repeat proteins with binding specificity for FAP were purified from E. coliculture (up to 200 mg ankyrin repeat protein per liter of culture) with a purity>95% as estimated from 15% SDS-PAGE. Representative examples of such SDS-PAGE gel are shown in FIG. 1A and FIG. 1B. Such purified ankyrin repeat proteins were used for further characterizations.

### Example 2: Determination of Dissociation Constants (K_{D}) of Ankyrin Repeat Proteins with Binding Specificity for FAP by Surface Plasmon Resonance (SPR) Analysis

The binding affinities of the purified ankyrin repeat proteins on the human FAP target were analyzed using a ProteOn instrument (BioRad) and the measurement was performed according standard procedures known to the person skilled in the art.

Briefly, human FAP was diluted in 10 mM Na-acetate pH 5.3 buffer and covalently immobilized on a GLC chip (BioRad) to a level of around 2000 resonance units (RU). The interaction of ankyrin repeat protein and hFAP was then measured by injecting 200 μl running buffer (PBS, pH 7.4 containing 0.005% Tween 20®) containing serial dilutions of ankyrin repeat proteins covering a concentration range between 30 nM and 0.5 nM (on-rate measurement), followed by a running buffer flow for at least 25 minutes at a constant flow rate of 100 μl/min (off-rate measurement). The regeneration was performed using 15 μl of 10 mM Glycine pH 2 followed by 15 μl of 124 mM H₃PO₄. The signals (i.e. resonance unit (RU) values) of the interspots and a reference injection (i.e. injection of running buffer only) were subtracted from the RU traces obtained after injection of ankyrin repeat protein (double-referencing). Based on the SPR traces obtained from the on-rate and off-rate measurements, the on- and off-rate of the corresponding ankyrin repeat protein—FAP interaction was determined.

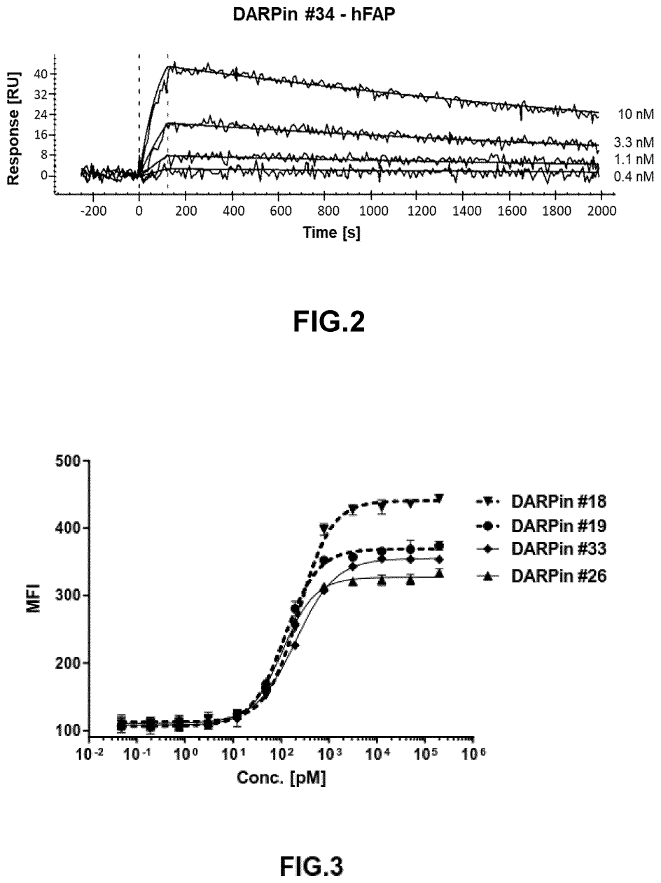

As a representative example, FIG. 2 shows the obtained SPR traces for DARPin® protein #34. Dissociation constants (K_{D}) were calculated from the estimated on- and off-rates using standard procedures known to the person skilled in the art. K_{D} values of selected ankyrin repeat proteins were determined to be in the range of 5 pM to 10 nM. Table 1 provides the K_{D} values of some selected ankyrin repeat proteins as examples.

TABLE 1
K_{D} values of ankyrin repeat protein - human FAP interactions

| DARPin ® protein # | K_{D} [nM] |
|---|---|
| #1 | 3.6 |
| #2 | 8.24 |
| #3 | 5.43 |
| #4 | 1.35 |
| #6 | 0.303 |
| #7 | 0.744 |
| #8 | 0.12 |
| #9 | 0.186 |
| #10 | 10.4 |
| #11 | 0.057 |
| #12 | 0.194 |
| #13 | 0.010 |
| #14 | 0.007 |
| #15 | <0.010 |
| #16 | 0.023 |
| #17 | 0.063 |
| #18 | 0.115 |
| #19 | 0.195 |
| #20 | 0.115 |
| #21 | 0.885 |
| #22 | 3.40 |
| #23 | 2.78 |
| #24 | 1.47 |
| #26 | 0.053 |
| #27 | 0.441 |
| #28 | 0.15 |
| #29 | 0.255 |
| #30 | 7.02 |
| #31 | 0.097 |
| #32 | 0.527 |
| #33 | 0.241 |
| #34 | 0.177 |
| #35 | 0.210 |
| #36 | 0.118 |
| #37 | 0.140 |
| #38 | 0.058 |
| #39 | 0.220 |

### Example 3: Binding of FAP-Specific Ankyrin Repeat Proteins on FAP+ Cells by FACS

Binding of purified FAP-specific ankyrin repeat proteins to FAP-expressing cells was analyzed by FACS.

Using multi-well plates, 20,000 WI38 cells were added per well in 50 μl of PBS. 50 μl of appropriate dilutions (2× dilution steps) of ankyrin repeat protein was then added to the cells and incubated for 30 minutes on ice. After the ankyrin repeat protein-binding reaction, cells were washed twice with PBS. Direct immunofluorescent detection of the His-tagged ankyrin repeat proteins using Penta-His AF647 conjugate (QIAgen) and determination of live cells using LIVE/DEAD™ Fixable Green Dead Cell Stain Kit (ThermoFisher) were then performed together in one step. The Penta-His AF647 conjugate and the LIVE/DEAD™ Fixable Green dye were applied to the cells together in 1/200 (Penta-His) and 1/1000 (LID Fixable) final dilutions and incubated for 20-30 minutes on ice. After washing the cells with PBS, cells were re-suspended in 1× CellFix™ buffer (BD Biosciences) (10× stock was diluted 1/10 in water). The samples were incubated in CellFix™ buffer for 15-20 minutes at RT, then spun down at 400 g for 5 min. Supernatant was discarded and cells were re-suspended in 200 μl PBS and stored at 4° C. until acquisition and FACS analysis. Acquisition and FACS analysis, including the determination of median fluorescence intensity (MFI), were done within 5 days after staining using an AttuneNxT instrument. Binding curves were fitted by non-linear regression curve fitting using GraphPad Prism software (v 7.0.4).

As a representative examples, FIG. 3 shows the obtained binding curves for DARPin® protein #18, DARPin® protein #19, DARPin® protein #26 and DARPin® protein #33. EC₅₀ values were determined using standard procedures known to the person skilled in the art. EC₅₀ values of selected ankyrin repeat proteins were determined to be in the range of 100 pM to 5 nM, demonstrating high affinity binding to FAP-expressing WI38 cells. Table 2 provides the EC₅₀ values of selected ankyrin repeat proteins as examples.

TABLE 2
EC₅₀ values on WI38 cells

| DARPin ® protein # | EC₅₀ [pM] |
|---|---|
| #1 | 3173 |
| #2 | 5257 |
| #3 | 5717 |
| #4 | 1086 |
| #5 | 654 |
| #7 | 1454 |
| #9 | 275.8 |
| #10 | 512 |
| #11 | 522.9 |
| #12 | 788.7 |
| #13 | 307 |
| #14 | 246.2 |
| #15 | 330.2 |
| #18 | 187.9 |
| #19 | 308.5 |
| #21 | 2185 |
| #22 | 1219 |
| #23 | 1819 |
| #24 | 4489 |
| #26 | 150.4 |
| #27 | 663.9 |
| #28 | 780.8 |
| #29 | 1131 |
| #30 | 650.5 |
| #32 | 918.4 |
| #33 | 205.5 |
| #36 | 234.8 |
| #37 | 117.6 |
| #38 | 106.1 |
| #39 | 202.5 |

Similarly, binding of the engineered ankyrin repeat proteins, i.e. DARPin® protein #34 and DARPin® protein #35, to FAP-expressing WI38 cells was also tested essentially as described above. Fold increase MFI was calculated by dividing the sample MFI by the MFI of the background control. For the background control, the binding reaction buffer was incubated only with the Penta-His AF647 conjugate, in the absence of a ankyrin repeat protein.

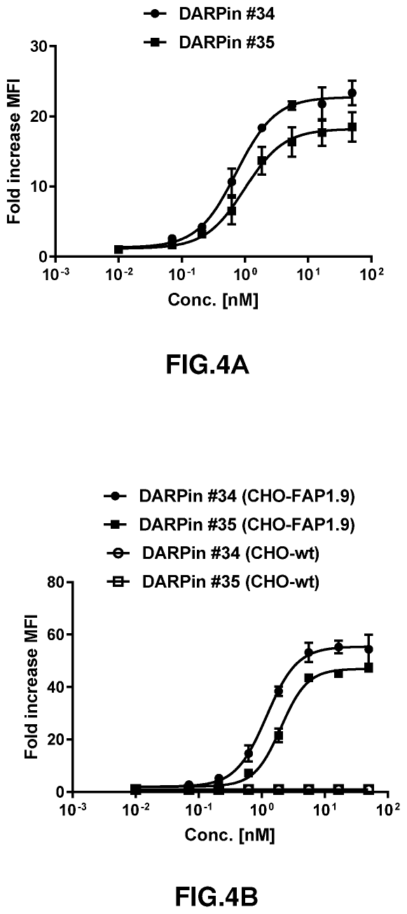

FIG. 4A shows the binding curves for DARPin® protein #34 and DARPin® protein #35 on WI38 cells, demonstrating that both DARPin® protein #34 and DARPin® protein #35 bind to FAP-expressing WI38 cells with high affinity. The EC₅₀ value for DARPin® protein #34 was 0.736 nM and the EC₅₀ value for DARPin® protein #35 was 0.994 nM (see Table 3).

In order to demonstrate that binding of FAP-specific ankyrin repeat proteins to FAP-expressing cells was indeed mediated by binding to FAP expressed on the cell surface, the binding of selected ankyrin repeat proteins to cells with or without FAP expression was measured. For this purpose, CHO cells were generated which express FAP on the cell surface. These FAP-expressing CHO cells can be used in comparison with wildtype CHO cells which do not express FAP.

In brief, an expression vector was generated by standard molecular biology techniques using a cDNA encoding human FAP (obtained from OriGene Technologies). CHO cells were transfected with the expression vector using Lipofectamine. Selection pressure was applied using different concentrations of Geneticin G-418 (Promega, V8091). Expression of hFAP was analysed by flow cytometry using an anti-FAP antibody. The population of CHO-hFAP transfectants obtained using 1.9 mg/mL G-418 was chosen for further experiments, based on a relatively low expression level of FAP in these cells (CHO-FAP1.9 cells). FACS analysis demonstrated that CHO-FAP1.9 cells but not wildtype CHO cells (CHO-wt) express hFAP on the cell surface (data not shown).

A series of dilutions of DARPin® protein #34 and DARPin® protein #35 were added to wildtype CHO cells (CHO-wt) and to hFAP expressing CHO cells (CHO-FAP1.9). The binding reaction and subsequent analysis and MFI measurements were performed essentially as described above for the WI38 cells. FIG. 4B shows the binding curves for DARPin® protein #34 and DARPin® protein #35 on CHO-wt cells and CHO-FAP1.9 cells, demonstrating that DARPin® protein #34 and DARPin® protein #35 bind only to those CHO cells (i.e. CHO-FAP1.9) that express FAP on the cell surface. Thus, binding of FAP-specific ankyrin repeat proteins to cells is mediated by binding to FAP expressed on the cell surface. The EC₅₀ values for binding to CHO-FAP1.9 cells were 1.186 nM for DARPin® protein #34 and 2.016 nM for DARPin® protein #35 (see Table 3).

TABLE 3
Binding of ankyrin repeat proteins to hFAP-expressing cells

| DARPin ® protein # | EC50 [nM] - WI38 | EC50 [nM] - CHO-FAP1.9 |
|---|---|---|
| #34 | 0.736 | 1.186 |
| #35 | 0.994 | 2.016 |

### Example 4: Binding to Cynomolgus FAP as Determined by SPR and cFAP Transfected CHO Cells

The binding of selected ankyrin repeat proteins to cynomolgus FAP (cFAP) was assessed on soluble cFAP target using a ProteOn instrument (BioRad) and on cellular cFAP target using transfected CHO cells and FACS according standard procedures known to the person skilled in the art.

Briefly, cyno FAP was diluted in 10 mM Na-acetate pH5.3 buffer and covalently immobilized on a GLC chip (BioRad) to a level of around 2000 resonance units (RU). The interaction of ankyrin repeat protein and cFAP was then measured by injecting 200 μl running buffer (PBS, pH 7.4 containing 0.005% Tween 20®) containing serial dilutions of ankyrin repeat proteins covering a concentration range between 25 nM and 0.3 nM (on-rate measurement), followed by a running buffer flow for at least 25 minutes at a constant flow rate of 100 μl/min (off-rate measurement). The regeneration was performed using 15 μl of 10 mM Glycine pH 2 followed by 15 μl of 124 mM H₃PO₄. The signals (i.e. resonance unit (RU) values) of the interspots and a reference injection (i.e. injection of running buffer only) were subtracted from the RU traces obtained after injection of ankyrin repeat protein (double-referencing). Based on the SPR traces obtained from the on-rate and off-rate measurements, the on- and off-rate of the corresponding ankyrin repeat protein—FAP interaction was determined.

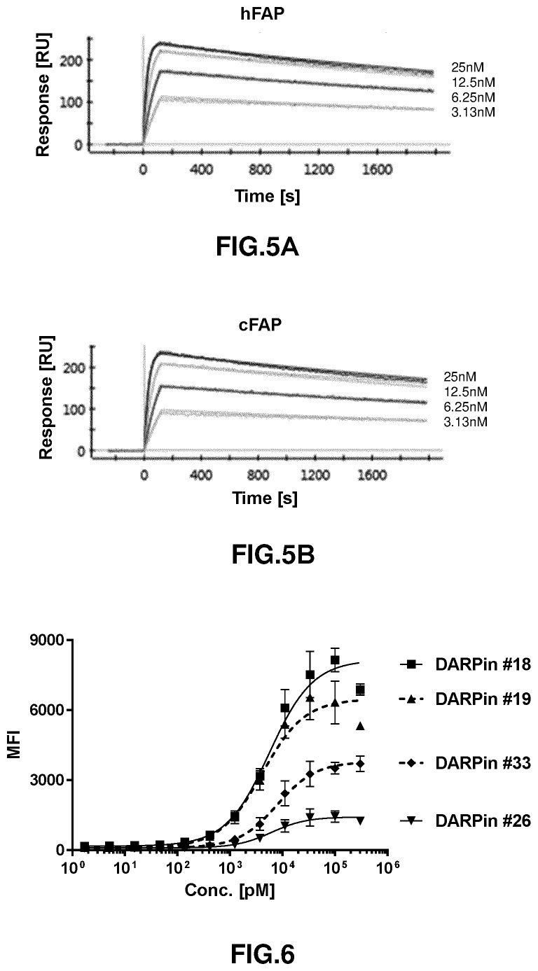

As representative examples, FIG. 5 shows the obtained SPR traces for DARPin® protein #36 on both cynomolgus and human FAP. Dissociation constants (K_{D}) were calculated from the estimated on- and off-rates using standard procedures known to the person skilled in the art. K_{D} values on cFAP were determined to be within a two-fold range of the K_{D} values on hFAP, demonstrating comparable binding of selected ankyrin repeat proteins to FAP proteins of these different species. Table 4 provides the K_{D} values on cFAP and hFAP of some of the selected ankyrin repeat proteins as examples.

TABLE 4
K_{D} values of DARPin - hFAP and DARPin - cFAP interactions

| DARPin ® protein # | K_{D} [pM] - hFAP | K_{D} [pM] - cFAP |
|---|---|---|
| #34 | 177 ± 93 | 71 |
| #35 | 210 | 150 |
| #36 | 118 ± 0 | 101 |
| #37 | 140 ± 17 | 112 |
| #38 | 58 ± 0.5 | 47 |
| #39 | 220 ± 21 | 155 |

To assess the binding of selected ankyrin repeat proteins to cynomolgus FAP (cFAP) expressed on the cell surface, stably transfected CHO cells expressing cFAP were generated essentially as described in Example 3 for hFAP, except that a cDNA encoding cFAP was used.

A series of dilutions of ankyrin repeat proteins were added to cFAP expressing CHO cells. The binding reaction and subsequent analysis and MFI measurements were performed essentially as described in Example 3 for the WI38 cells. FIG. 6 shows the binding curves for DARPin® protein #18, DARPin® protein #19, DARPin® protein #26 and DARPin® protein #33 on cFAP-expressing CHO cells, demonstrating that the FAP-specific ankyrin repeat proteins also bind to cynomolgus FAP expressed on the cell surface. The EC₅₀ values for binding to cFAP-expressing CHO cells are provided in Table 5.

TABLE 5
Binding of ankyrin repeat proteins to cFAP-expressing CHO cells

| DARPin ® protein # | EC₅₀ [nM] |
|---|---|
| #18 | 5.6 |
| #19 | 4.1 |
| #26 | 6.1 |
| #33 | 7.9 |

### Example 5: FAP-Expressing Cell Binding, SPR, Pharmacokinetics, Bio-Distribution, and Tumor Localization Data of FAP-Specific Ankyrin Repeat Proteins Fused to a Biologically Active Molecule

### Construction of FAP-Specific Ankyrin Repeat Proteins Fused to a Biologically Active Molecule

Selected FAP-specific ankyrin repeat domains were genetically fused to a biologically active molecule using standard molecular biology methods known to one of skill in the art. An ankyrin repeat domain with binding specificity for serum albumin (SEQ ID NO:38) was chosen as an exemplary biologically active molecule. Serum albumin-specific ankyrin repeat domains and their use for the in vivo half-life extension of covalently attached ankyrin repeat domains with different target specificities have been reported previously (see, e.g., Steiner et al., 2017, “Half-life extension using serum albumin-binding DARPin® domains,” Protein Eng. Des. Sel. 30(9):583-591 (2017); U.S. Pat. Nos. 9,284,361; 9,458,211). The ankyrin repeat domain of SEQ ID NO:38 binds to serum albumin of various species, including mouse, human and cynomolgus monkey. The biologically active molecule was connected to the FAP-specific ankyrin repeat domains via a peptide linker. A proline-threonine rich peptide linker (SEQ ID NO:39) was chosen as an exemplary peptide linker for this purpose.

The FAP-specific ankyrin repeat domains, together with the peptide linker and the biologically active molecule, were cloned into a pQE (QIAgen, Germany) based expression vector providing an N-terminal His-tag (SEQ ID NO:36) to facilitate simple protein purification, as described in Example 1. For example, expression vectors encoding the following ankyrin repeat fusion proteins were constructed:

DARPin® protein #40 (SEQ ID NO:40 with a His-tag (SEQ ID NO:36) fused to the N terminus). SEQ ID NO:40 comprises SEQ ID NO:38 and SEQ ID NO:18, connected by a peptide linker.

DARPin® protein #41 (SEQ ID NO:41 with a His-tag (SEQ ID NO:36) fused to the N terminus). SEQ ID NO:41 comprises SEQ ID NO:38 and SEQ ID NO:19, connected by a peptide linker.

DARPin® protein #42 (SEQ ID NO:42 with a His-tag (SEQ ID NO:36) fused to the N terminus). SEQ ID NO:42 comprises SEQ ID NO:38 and SEQ ID NO:26, connected by a peptide linker.

### Binding of FAP-Specific Ankyrin Repeat Proteins Fused to a Biologically Active Molecule to U87 MG and WI38 Cells

In order to determine whether the fusion of a biologically active molecule to a FAP-specific ankyrin repeat domain affects the ability of the molecule to specifically recognize and bind to FAP expressed on the surface of cells, the binding of selected ankyrin repeat fusion proteins to FAP-expressing cells was tested. U87 MG cells and WI38 cells are known to express FAP on the cell surface and they were chosen as representative cells for these experiments. FAP expression on the U87 MG and WI38 cells was confirmed by FACS analysis (data not shown).

Using multi-well plates, 40,000 cells (either U87MG or WI38) were added per well in 50 μl of PBS. 50 μl of appropriate dilutions (2× dilution steps) of ankyrin repeat fusion protein (DARPin® protein #40 or DARPin® protein #41) was then added to the cells and incubated for 30 minutes on ice. After the ankyrin repeat fusion protein-binding reaction, cells were washed twice with PBS. Direct immunofluorescent detection of the His-tagged ankyrin repeat fusion proteins using Penta-His AF647 conjugate (QIAgen) and determination of live cells using LIVE/DEAD™ Fixable Green Dead Cell Stain Kit (ThermoFisher) were then performed together in one step. The Penta-His AF647 conjugate and the LIVE/DEAD™ Fixable Green dye were applied to the cells together in 1/400 (Penta-His) and 1/1000 (LID Fixable) final dilutions and incubated for 20-30 minutes on ice. After washing the cells with PBS, cells were re-suspended in 1× CellFix™ buffer (BD Biosciences) (10× stock was diluted 1/10 in water). The samples were incubated in CellFix™ buffer for 15-20 minutes at RT, then spun down at 400 g for 5 min. Supernatant was discarded and cells were re-suspended in 200 μl PBS and stored at 4° C. until acquisition and FACS analysis. Acquisition and FACS analysis, including the determination of median fluorescence intensity (MFI), were done within 5 days after staining using an AttuneNxT instrument. Binding curves were fitted by non-linear regression curve fitting using GraphPad Prism software (v 7.0.4).

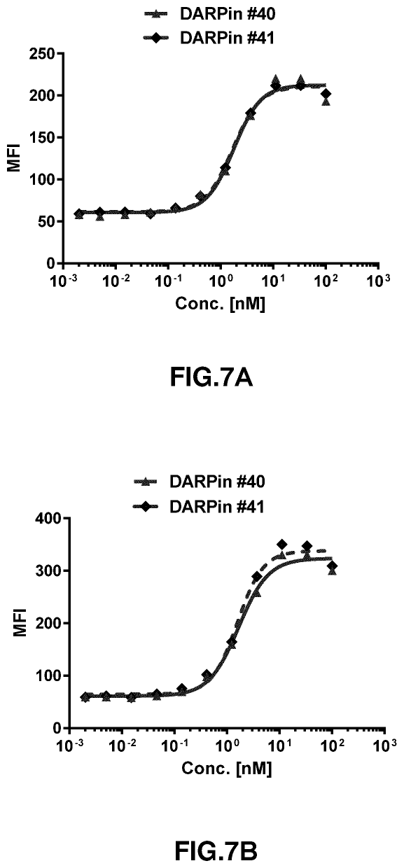

FIG. 7 shows the binding curves of DARPin® protein #40 and DARPin® protein #41 to the U87MG and WI38 cells, respectively. Both ankyrin repeat fusion proteins bound to the different cell types in a concentration-dependent manner, reaching binding saturation at a concentration between 10-100 nM. Thus, specific binding of the ankyrin repeat fusion proteins to cellular FAP is similar as compared to the FAP-specific ankyrin repeat domains without fusion to a biologically active molecule. The FAP-specific ankyrin repeat domains of the invention can therefore be fused to a biologically active molecule and such FAP-specific ankyrin repeat fusion proteins retain their ability to efficiently bind to FAP expressed on the surface of cells.

### Pharmacokinetics Profile of FAP-Specific Ankyrin Repeat Proteins Fused to a Biologically Active Molecule in Mice

In order to determine whether a FAP-specific ankyrin repeat domain of the invention can have an appropriate serum half-life in vivo for it to be useful for the development of therapeutic agents, the pharmacokinetic profiles of DARPin® protein #40, DARPin® protein #41 and DARPin® protein #42 were analyzed in mice.

### In Vivo Administration and Sample Collection

DARPin® protein #40, DARPin® protein #41 and DARPin® protein #42 were administered as a single intravenous bolus injection into the tail vein of 6 mice for each ankyrin repeat fusion protein. The target dose level was 1 mg/kg with an application volume of 5 mL/kg. Ankyrin repeat fusion proteins were formulated in phosphate-buffered saline (PBS) solution.

Mice were split into 2 groups with equal numbers of animals. Four serum samples were collected from each mouse. Blood samples for pharmacokinetic investigations were collected from the saphenous vein at 5 min, 4 h, 24 h, 48 h, 76 h, 96 h and 168 h post compound administration. Blood was kept at room temperature to allow clotting followed by centrifugation and collection of serum.

### Bioanalytics by ELISA to Measure Ankyrin Repeat Proteins in Serum Samples

One hundred μl per well of 10 nM polyclonal goat anti-rabbit IgG antibody (Ab18) in PBS was coated onto a NUNC Maxisorb ELISA plate overnight at 4° C. After washing with 300 μl PBST (PBS supplemented with 0.1% Tween20) per well five times, the wells were blocked with 300 μl PBST supplemented with 0.25% Casein (PBST-C) for 1 h at room temperature (RT) on a Heidolph Titramax 1000 shaker (450 rpm). Plates were washed as described above. 100 μl 5 nmol/L rabbit anti-DARPin® 1-1-1 antibody in PBST-C was added and the plates were incubated at RT (22° C.) with orbital shaking (450 rpm) for 1 h. Plates were washed as described above.

One hundred μl of diluted serum samples (1:20-1:312500 in 1:5 dilution steps) or ankyrin repeat protein standard curve samples (0 and 50-0.0008 nmol/L in 1:3 dilution steps) were applied for 2 h, at RT, shaking at 450 rpm. Plates were washed as described above.

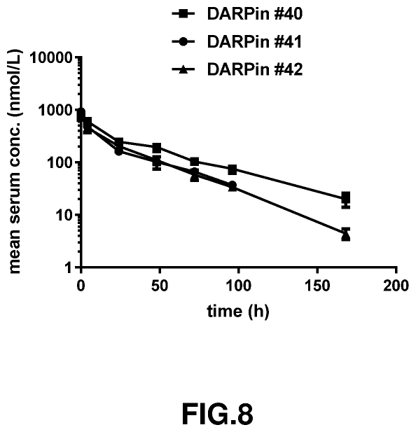

Wells were then incubated with 100 μl murine anti-RGS-His-HRP IgG (Ab06, 1:2000 in PBST-C) and incubated for 1 h, at RT, 450 rpm. Plates were washed as described above. The ELISA was developed using 100 μl/well TMB substrate solution for 5 minutes and stopped by the addition of 100 μl 1 mol/L H₂SO₄. The difference between the absorbance at 450 nm and the absorbance at 620 nm was calculated. Samples were measured in duplicate on two different plates. FIG. 8 shows the serum concentrations of DARPin® protein #40, DARPin® protein #41 and DARPin® protein #42 as a function of time after the single intravenous administration into mice. The traces indicate roughly mono-exponential elimination of the compounds.

### Pharmacokinetic Analysis

Pharmacokinetic data analysis was performed at Molecular Partners using Version 7.0 of the WinNonlin program as part of Phoenix 64, Pharsight, N.C. Calculation of the pharmacokinetic parameters based on the mean concentration-time data of the animals dosed via intravenous bolus injection was performed with non-compartmental analysis (NCA model 200-202, IV bolus, linear trapezoidal linear interpolation). The following pharmacokinetic parameters were calculated:

AUCinf, AUClast, AUC_% extrapol, Cmax, Tmax, CI_pred, Vss_pred, t½

Maximum serum concentrations (Cmax) and the times of their occurrence (Tmax) were obtained directly from the serum concentration-time profiles. The area under the serum concentration-time curve (AUCinf) was determined by the linear trapezoidal formula up to the last sampling point (Tlast) and extrapolation to infinity assuming mono-exponential decrease of the terminal phase. The extrapolation up to infinity was performed using Clast/Az, where Az denotes the terminal rate constant estimated by log linear regression and Clast denotes the concentration estimated at Tlast by means of the terminal log-linear regression. Total serum clearance (CI_pred) and the apparent terminal half-life were calculated as follows: CI_pred=i.v. dose/AUCinf and t½=In2/λz. The steady-state volume of distribution Vss was determined by: Vss=i.v. dose·AUMCinf/(AUCinf)2. AUMCinf denotes the total area under the first moment of drug concentration-time curve extrapolated to infinity using the same extrapolation procedure as described for calculation of AUCinf. To calculate PK parameters based on concentrations given in nmol/L dose values given as mg/kg were converted to nmol/kg by using the molecular weight of the ankyrin repeat proteins. Table 6 shows the pharmacokinetic data obtained for the tested ankyrin repeat fusion proteins.

TABLE 6
Pharmacokinetic data for DARPin ® #40, DARPin ® #41, and DARPin ® #42

| | | DARPin ® | DARPin ® | DARPin ® |
|---|---|---|---|---|
| Parameter | Unit | #40 | #41 | #42 |
| AUCINF_pred | h*(nmol/L) | 25779 | 17040 | 16613 |
| AUCINF_D_pred | (h*nmol*kg)/(L*mg) | 25779 | 17040 | 16613 |
| AUClast | h*(nmol/L) | 24649 | 15729 | 16440 |
| Cmax | nmol/L | 795 | 925 | 796 |
| Cmax_D | (nmol*kg)/(L*mg) | 795 | 925 | 796 |
| Tmax | h | 0.083 | 0.083 | 0.083 |
| Cl_pred | L/(h*kg) | 0.0012 | 0.0020 | 0.0018 |
| Vss_pred | L/kg | 0.059 | 0.065 | 0.063 |
| HL_Lambda_z | h | 38.8 | 26.9 | 26.1 |
| AUC_% Extrap_pred | (%) | 4 | 8 | 1 |
| AUC_% Back_Ext_pred | (%) | 0 | 0 | 0 |

The serum half-lives were determined as 38.8 hours for DARPin® protein #40, 26.9 hours for DARPin® protein #41, and 26.1 hours for DARPin® protein #42. These data indicate that FAP-specific ankyrin repeat domains of the invention have sufficient in vivo half-lives, when fused to a serum albumin-specific ankyrin repeat domain, to make them useful as components of therapeutic agents comprising a FAP-specific ankyrin repeat domain covalently connected to a biologically active molecule.

### Tumor Localization of FAP-Specific Ankyrin Repeat Protein Fused to a Biologically Active Molecule

In order to determine whether a FAP-specific ankyrin repeat domain of the invention can be used to localize a biologically active molecule preferentially to tumor tissue expressing FAP, the in vivo localization of a FAP-specific ankyrin repeat protein fused to a biologically active molecule was analyzed in a mouse tumor model.

### Binding of DARPin® Protein #41 to Mouse and Human FAP Measured by SPR

Before analyzing a FAP-specific ankyrin repeat fusion protein in a mouse tumor model, it has to be shown that the FAP-specific ankyrin repeat fusion protein binds not only to human FAP but also to mouse FAP (mFAP). For this purpose, binding of DARPin® protein #41 to soluble mouse and human FAP was assessed by SPR measurement using a ProteOn instrument (BioRad) according standard procedures known to the person skilled in the art.

Briefly, mouse FAP was diluted in 10 mM Na-acetate pH5.3 buffer and covalently immobilized on a GLC chip (BioRad) to a level of around 2000 resonance units (RU). The interaction of DARPin® protein #41 and mFAP was then measured by injecting 200 μl running buffer (PBS, pH7.4 containing 0.005% Tween20®) containing serial dilutions of DARPin® protein #41 covering a concentration range between 50 nM and 3 nM (on-rate measurement), followed by a running buffer flow for 30 minutes at a constant flow rate of 100 μl/min (off-rate measurement). The regeneration was performed using 15 μl of 10 mM Glycine pH 2 followed by 15 μl of 124 mM H₃PO₄. The signals (i.e. resonance unit (RU) values) of the interspots and a reference injection (i.e. injection of running buffer only) were subtracted from the RU traces obtained after injection of DARPin® protein #41 (double-referencing). The binding of DARPin® protein #41 to hFAP was determined as described in Example 2.

Dissociation constants (K_{D}) were calculated from the estimated on- and off-rates using standard procedures known to the person skilled in the art and are reported in Table 7. These results show that the FAP-specific ankyrin repeat domain fused to a biologically active molecule (DARPin® protein #41) binds to human FAP with similar K_{D} as the corresponding FAP-specific ankyrin repeat domain alone (see DARPin® protein #19 in Table 1) and that it cross-reacts well with mouse FAP. The dissociation constant of DARPin® protein #41 for binding to mouse FAP was determined to be 1.3 nM.

TABLE 7
K_{D} values of DARPin ® - hFAP and DARPin ® -
mFAP interactions

| DARPin ® protein # | K_{D} [nM] - hFAP | K_{D} [nM] - mFAP |
|---|---|---|
| #41 | 0.264 | 1.3 |

### In Vivo Localization of DARPin® Protein #41 Assessed in a FAP-Positive Mouse Tumor Model by Biodistribution Analysis

A mouse tumor model involving U87 MG glioblastoma cells was chosen as a representative FAP-positive tumor model for the assessment of the in vivo localization of DARPin® protein #41. U87 MG cells express FAP on their cell surface and they are bound by FAP-specific ankyrin repeat domains of the invention (see above). Furthermore, U87 tumors in mice also express FAP, as expected (data not shown).

10 million U87 MG cells per mouse were engrafted subcutaneously in female CD1 (nu/nu) mice. The cells were engrafted at two separate locations (5 million cells per location), giving rise to two tumors per mouse. The engrafted tumor cells were allowed to proliferate and the tumors were allowed to grow for 3-4 weeks until a tumor volume of 500 mm³ was reached and before ankyrin repeat fusion protein was administered. To prepare the ankyrin repeat fusion protein for administration, DARPin® protein #41 was radioactively labeled with 99m-Technetium by complex formation with the His-tag of DARPin® protein #41, according to methods known in the art (see, e.g., Waibel et al., Nature Biotech. 17: 897-901 (1999); Egli et al., J. Nucl. Med. 40: 1913-1917 (1999)). As a control compound, a protein (DARPin® protein #43; SEQ ID NO:43) was used that is identical to DARPin® protein #41, except that the FAP-specific ankyrin repeat domain was replaced with a non-binding ankyrin repeat domain (i.e. an ankyrin repeat domain that does not bind to FAP and has no known binding specificity). DARPin® protein #43 was similarly labeled with 99m-Technetium as DARPin® protein #41.

30 female U87-tumor-bearing CD1 (nu/nu) mice were divided into two groups. One group received a single dose of ˜3.6 MBq (˜1 mg/kg) of DARPin® protein #41 by injection into the tail vein. The other group received a single dose of ˜3.6 MBq (˜1 mg/kg) of DARPin® protein #43 by injection into the tail vein. Biodistribution was monitored at time points 1 h, 24 h and 48 h after injection of the radioactively labeled ankyrin repeat fusion proteins. Mice were anesthetized and killed by cervical dislocation (5 mice per time point) and aliquots of blood were collected. The organs of interest (including, e.g., tumor, spleen, kidney, liver, muscle) were extracted, weighed and the radioactivity was determined with a γ-counter (Packard Cobra II Gamma D5010, GMI, USA). The ratios of the measured radioactivity in different organs to the measured radioactivity in blood were calculated.

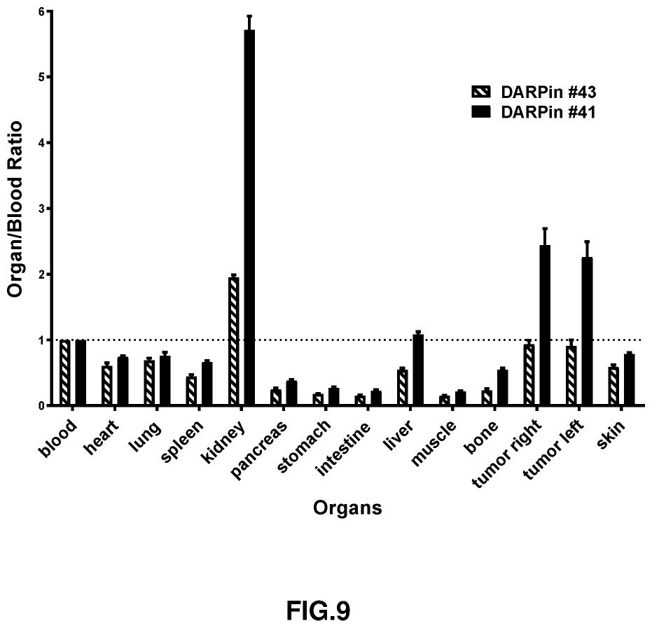

FIG. 9 shows the biodistribution of DARPin® protein #41 and the control compound DARPin® protein #43 at 48 hours post-injection. High organ/blood ratios of radioactivity were observed for DARPin® protein #41 in the kidney and in the two tumors, but not in other organs. For the control DARPin® protein #43, an elevated organ/blood ratio of radioactivity was observed in the kidney, but not in the two tumors or other organs. The elevated levels of radioactivity observed for DARPin® protein #41 and DARPin® protein #43 in the kidney are expected, since the kidney is likely a site of DARPin® molecule elimination. The elevated levels of radioactivity observed for DARPin® protein #41 in the two tumors indicate that FAP-targeting of DARPin® protein #41 resulted in high tumor accumulation and retention. Considering the serum half-live of DARPin® protein #41 (see above) and the expected clearance of DARPin® protein #41 from the circulation, the high tumor/blood ratios observed for DARPin® protein #41 at 48 hrs post-injection also indicate an extended tumor retention due to FAP-targeting. Taken together, the results of the biodistribution analysis show that FAP-specific ankyrin repeat domains of the invention can be fused to a biologically active molecule (such as, e.g., the serum albumin-binding domain in DARPin® protein #41) and used to localize and retain the biologically active molecule in FAP-expressing tumor tissue. Thus, FAP-specific ankyrin repeat domains of the invention can be used to localize and retain therapeutically active molecules (such as anti-tumor agents) in FAP-expressing tumors and thereby to reduce potential side effects of the therapeutically active molecules in other organs and the organism as a whole.

### Example 6: FAP-Specific Ankyrin Repeat Protein Fused to a Biologically Active Molecule Inhibits Tumor Growth and Selectively Increases Density of Human CD8 T Cells in the Tumor

The ability of a FAP-specific ankyrin repeat protein linked to a biologically active molecule (an immune modulator) to stimulate T cells and inhibit tumor growth in vivo was tested in a HT-29 colon carcinoma xenograft model reconstituted with human peripheral blood mononuclear cells (PBMCs) (MiXeno). This humanized mouse model has been described as suitable for testing immune stimulatory efficacy of immune checkpoint and co-stimulatory drugs. The model was used to assess whether a binding protein of the invention comprising a FAP-specific ankyrin repeat domain and a biologically active molecule was able to increase intra-tumoral T cell infiltration and slow tumor growth. The binding protein used for the experiment (DARPin® protein #45) comprised SEQ ID NO: 34 connected at its C-terminus by a peptide linker (SEQ ID NO:39) to an agonistic T-cell costimulatory receptor (TCCR)-specific ankyrin repeat protein as biologically active molecule. The molecular identity of the TCCR is disclosed in U.S. provisional patent application No. 62/857,037 entitled “Multispecific Proteins” filed at the U.S. Patent & Trademark Office on 4 Jun. 2019 and assigned to Molecular Partners AG, and in the PCT international patent application claiming priority from U.S. 62/857,037 and filed on the filing date of the instant PCT application. TCCRs and other immune modulators are well known in the art (see, e.g., Smith-Garvin et al., Annu Rev Immunol. 27: 591-619 (2009)). Treatment with a monoclonal antibody against the targeted TCCR in this model was sufficient to significantly slow tumor growth, but it also induced strong systemic effects such as accelerated graft versus host disease (GVHD) and liver T cell infiltration resulting in premature death compared to untreated mice.

### Materials and Methods:

Tumor experiment: Immunodeficient NOG mice were inoculated subcutaneously in the right flank region with HT-29 tumor cells (3.5×10⁶). The mice were then humanized by injecting peripheral blood mononuclear cells (PBMCs) from two healthy human donors (3.5×10⁶ cells/mouse). The test articles were administered to the tumor-bearing mice according to the predetermined regimen as shown in Table 8.

TABLE 8
Study design - experimental groups

| | | Inoculum | | | | | |
|---|---|---|---|---|---|---|---|
| | | | PBMC 7 × 10⁶/ | | Dose | Dosing | Actual |
| Group | N | HT-29 (s.c.) | mouse (i.p.) | Treatment | (mg/kg) | Route | Schedule |
| 1 | 5 | 3.5 × 10⁶ cells | Donor 1 | Vehicle | 0 | i.v. | Q2D × 10 |
| | 5 | | Donor 2 | | | | |
| 2 | 5 | | Donor 1 | DARPin ® | 0.32 | i.v. | Q2D × 10 |
| | 5 | | Donor 2 | protein #45 | | | |

Date of tumor cell and PBMC inoculation was denoted as day 0. Tumor growth was monitored every 3 to 4 days. On day 18 of the experiment, mice were sacrificed, tumors removed, and studied by flow cytometry and quantitative immunofluorescence (QIF). Tumor growth analyses were limited to 18 days because mice started to show signs of graft-versus-host-disease (GVHD) after this time.

Flow cytometry: Data from raw FCS files were analyzed with FlowJo software (TreeStar). Cells were gated on live lymphocytes expressing the human surface markers CD45, CD4 and CD8. Dead cells were excluded from the analysis via incorporation of the live-dead labelling dye 7-AAD. Shown are the percentages of human CD8 T cells as percentage of total human CD45 positive cells detected in blood.

Immunohistochemistry: Tissues were recovered from mice at necropsy, and embedded in optimum cutting temperature compound (Sakura) and frozen without prior fixation. OCT embedded cryo-preserved specimens were cut into 7 μm sections and mounted on glass slides. The slides were fixed with cold acetone. The multiple immunofluorescence staining was performed with the following antibodies: anti-CD4 (Goat Pab, R&D System #AF-379-NA), anti-CD8 (Rabbit PAb, Abcam #ab40555) and anti-CD45 (clone H130, Biolegend #304002). These antibodies were respectively detected by anti-Sheep-Alexa Fluor® 647 (Thermofisher #A21448), anti-rabbit-Rhodamine Red™-X (Jackson ImmunoResearch #711-296-152) and anti-mouseIgG1-Alexa Fluor® 488 (Jackson ImmunoResearch #115-545-205). The images were acquired on a Zeiss Axio Scan.Z1 slidescanner. The images were transferred with Zen blue software and analyzed using ImageJ 1.51n software, with FIJI package to quantify numbers of human CD45, CD8 and CD4 T cells.

Statistical Analysis: Statistical analyses were performed with the Prism 7.0.2 software (GraphPad Software). Tumor growth and body weight data were analyzed for statistically significant differences by using repeat measurement two-way ANOVA and Tukey's multiple comparison test (GraphPad Prism, Vers. 7.02). Survival curves were analyzed by the Kaplan-Meier method and compared by log-rank tests. Flow cytometry data at study end were analyzed using 1-way ANOVA (GraphPad Prism, Vers. 7.02). A two-tailed P<0.05 was considered as statistically significant.

### Results

Tumor growth: Tumor growth was followed individually over time. In addition to the statistical analysis conducted on the data obtained at day 18 after tumor inoculation using Independent-Samples T test, tumor growth data were analyzed for statistically significant differences by using repeat measurement two-way ANOVA followed by Tukey's multiple comparison test. The tumor growth inhibition is summarized in Table 9.

TABLE 9
Summary of Anti-tumor Activity of Treatment

| | | Tumor Size | | | |
|---|---|---|---|---|---|
| | | at day 18 | TGI | Signif- | P |
| Group | Treatment | (mm³)^{a} | (%) | icance^{b} | value^{b} |
| 1 | Vehicle | 477 ± 56 | — | | — |
| 2 | DARPin | 349 ± 28 | 27 | ** | 0.0075 |
| | protein #45 | | | | |

^{a}Mean ± SEM;
^{b}RM two-way ANOVA over all time points of tumor growth curves followed by Tukey's multiple comparison test vs. vehicle control (* p < 0.05, ** p < 0.001).

Analysis of the entire tumor growth curves gives higher power to the analysis compared to analysis of only the final tumor volume at the end of the study. The two analyses correlate well. Tumor growth was delayed in the DARPin® protein #45 treatment group (p<0.001). The vehicle administered had no significant impact on tumor growth. In summary, the test substance, DARPin® protein #45, demonstrated significant anti-tumor activity in the subcutaneous HT-29 human colon cancer MiXeno model.

Immunophenotyping of Blood and Tumors: To confirm the results obtained by flow cytometry, the human CD4 and CD8 T lymphocyte density was analyzed by histology in tumors excised on day 18. Histological examination was performed using tissues from 5 mice per group (data not shown). Treatment with DARPin® protein #45 led to denser infiltrates of human CD8 T lymphocytes in comparison with the vehicle group. The difference reached significance (P<0.01). On the other hand, numbers of CD4 tumor infiltrating lymphocytes were not significantly different across the groups.

Histological Analysis of Liver T Cell Infiltration: Histologic examination of livers excised on day 18 was performed using tissues from 5 mice per group. Quantification of infiltrates categorized as small, medium and large by surface area showed that treatment with DARPin® protein #45 did not induce an increase in liver T cell infiltration. This is in contrast to published results showing that administration of an anti-TCCR monoclonal antibody induced increased liver T cell infiltration by human PBMCs in NOG mice. Treatment with DARPin® protein #45 also did not induce accelerated graft versus host disease (GVHD) or cause premature death compared to untreated mice.

### CONCLUSION

DARPin® protein #45 demonstrated anti-tumor activity in the subcutaneous HT-29 human colon cancer MiXeno model. Treatment with DARPin® protein #45 led to an increased density of human CD8 T cells in the tumor compared to vehicle-treated mice. Treatment with DARPin® protein #45 was well tolerated and did not lead to body weight loss or reduced survival and did not produce increased liver T cell infiltration compared to the vehicle group.

### Example 7: Characterization of FAP Functional Activity Upon Binding of FAP-Specific Ankyrin Repeat Proteins

To evaluate the effects of ankyrin repeat binding on the functional activity of FAP, selected FAP-specific ankyrin repeat proteins were tested for their ability to inhibit FAP enzymatic activity. To this end, the prolyl endopeptidase activity of FAP on a fluorogenic substrate was measured in the presence and in the absence of FAP-specific ankyrin repeat proteins.

Briefly, human FAP protein, the Z-Gly-Pro-AMC fluorogenic substrate (Bachem) and an excess of FAP-specific ankyrin repeat proteins were diluted in assay buffer (50 mM Tris, 1 M NaCl, 1 mg/mL BSA, pH 7.5) and added to a F16 Black Maxisorp plate at final concentrations of 0.1 μl/ml of human FAP, 50 pM of substrate and a 112-fold molar excess of ankyrin repeat protein (over hFAP) for a total final volume of 100 μl per well. After 75 min at room temperature, the cleavage of the substrate by hFAP was measured on a Tecan 1000 reader using excitation and emission wavelengths of 380 nm and 460 nm, respectively. 100 μl of 50 μM substrate was loaded for substrate blank (as a background control) and samples without ankyrin repeat protein (replaced by assay buffer) were used to determine the maximal activity of FAP. After background normalization, the percentage of FAP activity left in the presence of a given ankyrin repeat protein was determined based on the ratio of the FAP activity in presence of the ankyrin repeat to the maximal FAP activity. The results for a selected panel of FAP-specific ankyrin repeat proteins are summarized in Table 10. None of the selected ankyrin repeat proteins significantly inhibited FAP enzymatic activity.

TABLE 10
FAP activity in presence of ankyrin repeat protein

| DARPin ® protein # | FAP activity (%) |
|---|---|
| #1 | 115 |
| #2 | 117 |
| #3 | 116 |
| #4 | 111 |
| #5 | 103 |
| #6 | 146 |
| #7 | 123 |
| #8 | 97 |
| #9 | 115 |
| #10 | 120 |
| #11 | 117 |
| #12 | 114 |
| #13 | 88 |
| #14 | 86 |
| #15 | 93 |
| #16 | 97 |
| #17 | 96 |
| #18 | 112 |
| #19 | 94 |
| #20 | 104 |
| #21 | 108 |
| #22 | 100 |
| #23 | 100 |
| #24 | 102 |
| #25 | 110 |
| #26 | 106 |
| #27 | 110 |
| #28 | 103 |
| #29 | 109 |
| #30 | 101 |
| #31 | 123 |
| #32 | 138 |
| #33 | 102 |
| #34 | 93 |
| #35 | 90 |

1. A recombinant binding protein comprising an ankyrin repeat domain, wherein said ankyrin repeat domain has binding specificity for fibroblast activation protein (FAP), and wherein said ankyrin repeat domain comprises an ankyrin repeat module comprising an amino acid sequence selected from the group consisting of (1) SEQ ID NOs: 48 to 134 and (2) one or more sequences in which up to 9 amino acids in any of SEQ ID NOs: 48 to 134 are exchanged by another amino acid.

2. The binding protein of claim 1, wherein said ankyrin repeat module comprises an amino acid sequence selected from the group consisting of (1) SEQ ID NOs: 94 to 98, 111 to 113 and 132 to 134 and (2) one or more sequences in which up to 9 amino acids in any of SEQ ID NOs: SEQ ID NOs: 94 to 98, 111 to 113 and 132 to 134 are exchanged by another amino acid.

3. The binding protein of claim 1, wherein said ankyrin repeat module is a first ankyrin repeat module and comprises an amino acid sequence selected from the group consisting of (1) SEQ ID NO: 94 and (2) one or more sequences in which up to 9 amino acids in SEQ ID NO: 94 are exchanged by another amino acid, and wherein said ankyrin repeat domain further comprises a second ankyrin repeat module comprising an amino acid sequence selected from the group consisting of (1) SEQ ID NO: 95 and (2) one or more sequences in which up to 9 amino acids of SEQ ID NO: 95 are exchanged by another amino acid, and wherein said ankyrin repeat domain further comprises a third ankyrin repeat module comprising an amino acid sequence selected from the group consisting of (1) SEQ ID NO: 96 and (2) one or more sequences in which up to 9 amino acids of SEQ ID NO: 96 are exchanged by another amino acid.

4. The binding protein of claim 3, wherein said first ankyrin repeat module is located N-terminally of said second ankyrin repeat module within said ankyrin repeat domain, and wherein said second ankyrin repeat module is located N-terminally of said third ankyrin repeat module within said ankyrin repeat domain.

5. A recombinant binding protein comprising an ankyrin repeat domain, wherein said ankyrin repeat domain has binding specificity for fibroblast activation protein (FAP), and wherein said ankyrin repeat domain comprises an amino acid sequence with at least 80% amino acid sequence identity with any one of SEQ ID NOs: 1 to 35 and 144 to 153, wherein G at position 1 and/or S at position 2 of SEQ ID NOs: 1 to 35 and 144 to 153 are missing, and wherein L at the second last position and/or N at the last position of SEQ ID NOs: 1 to 33, 144, 145 and 148 to 150 are exchanged by A.

6. The binding protein of claim 5, wherein said ankyrin repeat domain comprises the amino acid sequence of SEQ ID NO: 18, wherein G at position 1 and/or S at position 2 of said ankyrin repeat domain are missing, and wherein L at the second last position and/or N at the last position of SEQ ID NO: 18 are exchanged by A.

7. The binding protein of claim 5, wherein said ankyrin repeat domain comprises the amino acid sequence of SEQ ID NO: 34, wherein G at position 1 and/or S at position 2 of said ankyrin repeat domain are missing.

8. The binding protein of claim 1, wherein said ankyrin repeat domain binds human FAP in PBS with a dissociation constant (Kd) below 10⁻⁷M.

9. The binding protein of claim 1, wherein said binding protein further comprises a biologically active molecule.

10. A nucleic acid encoding the binding protein of claim 1.

11. A pharmaceutical composition comprising the binding protein of claim 1, and a pharmaceutically acceptable carrier and/or diluent.

12. A method of localizing a biologically active molecule to FAP-expressing cells or tissue in a mammal, the method comprising administering to said mammal the binding protein of claim 9.

13. A method of treating a medical condition, the method comprising the step of administering to a patient in need thereof a therapeutically effective amount of the binding protein of claim 9, wherein said biologically active molecule is a therapeutically effective molecule.

14. The method of claim 12, wherein said FAP-expressing cells or tissue are located in a tumor.

15. A method of imaging a tumor and/or diagnosing a cancer in a patient, the method comprising the step of administering to a patient the binding protein of claim 9, wherein said biologically active molecule is a molecule effective for imaging cells or tissue bound by said binding protein.

16. The binding protein of claim 1, wherein said ankyrin repeat domain binds to human FAP-expressing WI38 cells with an EC₅₀ below 10⁻⁸M.

17. The binding protein of claim 1, wherein binding of said ankyrin repeat domain to FAP does not inhibit the prolyl endopeptidase activity of FAP by more than 25%.

18. The binding protein of claim 8, wherein said ankyrin repeat domain binds to human FAP-expressing WI38 cells with an EC₅₀ below 10⁻⁸M and binding of said ankyrin repeat domain to FAP does not inhibit the prolyl endopeptidase activity of FAP by more than 25%.

19. A pharmaceutical composition comprising the nucleic acid of claim 10, and a pharmaceutically acceptable carrier and/or diluent.

20. The method of claim 13, wherein said FAP-expressing cells or tissue are located in a tumor.
